(19) United States
(12) Patent Application Publication
(10) Pub. No.: US 2019/0170739 A1
(43) Pub. Date: Jun. 6, 2019

(54) ELECTROCHEMICAL DETECTION SYSTEMS AND COMPONENTS THEREOF

(71) Applicant: Laxmi Therapeutic Devices, Inc., Goleta, CA (US)

(72) Inventors: Logan Earl Garner, Westminster, CO (US); Luke Satish Kumar Theogarajan, Goleta, CA (US); William Van Antwerp, Valencia, CA (US)

(21) Appl. No.: 16/209,649

(22) Filed: Dec. 4, 2018

Related U.S. Application Data
(60) Provisional application No. 62/594,197, filed on Dec. 4, 2017.
(60) Provisional application No. 62/758,259, filed on Nov. 9, 2018.

(51) Int. Cl. G01N 33/543; G01N 27/416; C12Q 1/00; C08L 33/24; A61B 5/145; A61L 31/14; G01N 27/327

(52) U.S. Cl. CPC G01N 33/54313; G01N 27/4166; G01N 33/5438; C12Q 1/001; C08L 2203/02; A61B 5/14514; A61L 31/145; G01N 27/3273; G01N 2333/902; C08L 33/24

(57) ABSTRACT

The invention is directed to electrochemical detection systems, components thereof, materials and methods for making the systems and components thereof, and methods for detecting analytes with the systems. The systems include electrochemical cells containing hydrogels. The hydrogels include polymers with amine-containing pendant groups. The polymers can be cross-linked via the amine-containing pendant groups. The polymers can also or alternatively include enzymes and/or electron shuttles tethered thereto via amine-containing pendant groups. Monomers for making the polymers are provided. The systems can be used to detect analytes such as glucose.

### FIELD OF THE INVENTION

The invention is directed to electrochemical detection systems, components thereof, materials and methods for making the systems and the components thereof, and methods of detecting analytes with the systems.

### BACKGROUND

The quantitative determination of analytes in bodily fluids is important for the diagnoses and maintenance of certain physiological abnormalities. For example, lactate, cholesterol, and bilirubin should be monitored in certain individuals. In particular, it is important that diabetic individuals frequently check the glucose level in their body fluids to regulate the glucose intake in their diets. The results of such tests can be used to determine what, if any, insulin or other medication needs to be administered.

Electrochemical systems have been used for detecting analytes in a sample. These systems, however, have difficulty detecting analytes in small volumes, such as in the nanoliter and picoliter range. Materials suitable for detecting analytes in small sample volumes are needed.

### SUMMARY OF THE INVENTION

An exemplary aspect of the invention includes an electrochemical detection system based on functional hydrogel materials containing immobilized oxidoreductase enzymes. The system can be used for detecting analytes such as glucose. The general detection system architecture includes a two- or three-electrode electrochemical cell functionalized with the enzyme-containing hydrogel material. Sensing is based upon direct or indirect measurement of electrons from enzymatic analyte oxidation. Signal transduction, in the form of electrochemical response that is directly correlated with analyte concentration, is facilitated by the hydrogel material which serves to immobilize the enzyme component in close proximity to the electrodes, regulate mass transport rates of analytes, and, in some versions of the invention, mediate transport of electrons from oxidoreductase enzyme to electrode. The features of the hydrogel materials result in a detection system that is amenable to multiple detection modes that provide the ability to tune operating voltages, maximize sensitivity, and minimize background signal. The disclosed detection system drastically reduces the minimum sample volume to the nanoliter and even picoliter range, which is well below the present state of the art.

Another exemplary aspect of the invention includes versatile hydrogel and redox hydrogel functional materials containing oxidoreductase enzymes that can be used for next generation electrochemical enzymatic biosensors. The materials include hydrophilic polymers, hydrogels, and redox hydrogels. The polymers can be prepared from acrylamide/methacrylamide and modified acrylamide/methacrylamide co-monomers equipped with pendants bearing amine and cationic ammonium functional groups. The amine and ammonium groups act as sites for functionalization (i.e., tuning of materials to achieve desired traits or sites that facilitate desired processes/modifications) and/or that undergo desired or favorable interactions. The polymers include chains of hydrophilic repeating units decorated with the amine, cationic ammonium or other functional groups that allow the polymers to be modified for a wide range of specific applications. Long and flexible pendants facilitate favorable reactions and interactions by endowing the amine and ammonium groups with increased amplitude of motion relative to such groups confined in close proximity to the polymer backbone. The multifunctional hydrogel materials disclosed herein address the materials-based needs of next generation electrochemical enzymatic sensing systems. The materials disclosed herein have a high degree of versatility, which provides functionality for addressing the majority of challenges associated with the development of next generation sensing technologies.

Another exemplary aspect of the invention includes acrylamide-, alkylarylamide-, acrylate-, and alkylacrylate-based monomer building blocks equipped with pendant oligo(ethylene glycol) chains bearing terminal nitrogen-containing functional groups, and methods of preparing same. The monomers are water-soluble monomers and can serve as building blocks of hydrophilic polymer-based functional materials for the hydrogels and enzymatic biosensing systems described above. The monomers can be prepared in very few synthetic steps using mild conditions and can be readily equipped with a wide range of functional groups. The monomers can be functionalized with a variety of linking groups prior to undergoing polymerization.

Another exemplary aspect of the invention includes redox mediators, including electron shuttles, that can be used in the systems and detection methods of the invention.

Another exemplary aspect of the invention includes methods of detecting an analyte, such as glucose with the systems described herein.

Other exemplary aspects of the invention include methods of making the systems, polymers, monomers, and redox mediators of the invention.

The objects and advantages of the invention will appear more fully from the following detailed description of the preferred embodiment of the invention made in conjunction with the accompanying drawings.

### BRIEF DESCRIPTION OF THE DRAWINGS

FIGS. 1A-1D show generalized structures of exemplary monomer building blocks of polymers of the invention. R¹ in each instance can independently be H, alkyl, cycloalkyl, aryl, heterocycle, heteroaryl, or a combination thereof. R² in each instance can independently N or O. R³ in each instance can independently be, when R² is N: H; alkyl, cycloalkyl, aryl, heterocycle, heteroaryl, or a combination thereof; or a nitrogen protecting group. R³ is absent when R² is O. R⁴ in each instance can be a spacer arm. R⁶, R⁷, and R⁸ in each instance can independently be H; alkyl, cycloalkyl, aryl, heterocycle, heteroaryl, or a combination thereof; a nitrogen protecting group; a tethered polypeptide; a tethered redox mediator; or a linking arm, with the proviso that at least one of R⁶, R⁷, and R⁸ may be absent.

FIG. 2A shows schemes for preparing monomers having various tethered linking groups prior to polymer polymerization. The various R groups can be as defined for FIGS. 1A-D. FIG. 2B shows various linking groups that can constitute R¹¹ in FIG. 2A. R in FIG. 2B can be H; alkyl, cycloalkyl, aryl, heterocycle, heteroaryl, or a combination thereof.

FIGS. 3A-3G show various exemplary monomers that can be polymerized to generate polymers of the invention. The depicted counterions can be replaced with any counterion, such as chloride, bromide, etc. FIGS. 3F (tetra(ethylene glycol) diacrylate (TEGDA)) and 3G (polyethylene glycol dimethylacrylamide) can serve as polymerizable cross-linkers. Synthesis schemes for generating the monomers depicted in FIGS. 3D and 3E are shown below in FIGS. 4A and 4B, respectively. Synthesis schemes for generating the monomers shown in FIGS. 3A-3C are described elsewhere herein.

FIGS. 4A-4B show schemes for synthesizing the monomers of FIGS. 3D and 3E, respectively.

FIGS. 5A-5I show base structures (no tethering functionality or substitutions shown) of exemplary electron shuttles. FIG. 5A depicts a ferrocene electron shuttle. FIG. 5B depicts a 1,4-naphthoquinone electron shuttle. FIG. 5C depicts an anthroquinone electron shuttle.

FIG. 5D depicts a tetrathiafulvalene electron shuttle. FIG. 5F depicts a tetracyanoquinodimethane electron shuttle. FIG. 5F depicts a 1-methylphanazine electron shuttle. FIG. 5G depicts a 2,6-dichlorophenolindophenol electron shuttle. FIG. 5H depicts an indigo carmine electron shuttle. FIG. 5I depicts a methylene blue electron shuttle.

FIG. 6 shows examples of soluble and tethered 1,2-naphthoquinones as exemplary electron shuttles.

FIGS. 7A and 7B show schemes for synthesizing exemplary 1,2-naphthoquinones for use as electron shuttles. R¹⁶ can be H, alkyl, or perfluoroalkyl. R¹⁷ can be H, alkyl, cycloalkyl, aryl, heterocycle, heteroaryl, or a combination thereof; a tethered polymer; a tethered monomer; or a linking arm. R¹⁷ can be tethered to a polymer monomer or a polymer via any one of R⁶, R⁷, and R⁸ in the polymers and monomers provided herein.

FIG. 8 shows an exemplary reaction for generating a 1,2-naphthoquinone electron shuttle comprising a tether with an unreacted linking group.

FIGS. 9A-9D show structures of exemplary tethering agents. FIG. 9A depicts glutaraldehyde as a tethering agent. FIG. 9B depicts a tethering agent including N-hydroxysuccinimide and maleimide terminal linking groups and an alkyl chain spacer arm.

FIG. 9C depicts a tethering agent including N-hydroxysuccinimide and maleimide terminal linking groups and a polyethylene glycol spacer arm. FIG. 9D depicts a tethering agent including diglycidyl terminal linking groups and a polyethylene glycol spacer arm. In each instance, n can be 0-20 or more.

FIG. 10 shows generalized structures of exemplary amine-reactive functional groups.

FIGS. 11A-11C show exemplary corresponding linking groups. For full references of the citations in FIGS. 11A-11C, see Patterson et al., ACS Chem. Biol. 2014, 9, 592-605.

FIG. 12A shows a schematic cartoon representation for (A) enzyme tethering post-polymerization; (B) enzyme tethering and electron shuttle installation post-polymerization; and (C) enzyme tethering, electron shuttle installation, and cross-linking (via polymer backbone-to-polymer backbone tethers) to form a hydrogel starting from a mixture of enzyme and linear polymer.

FIG. 12B shows a schematic cartoon representation for electron shuttle installation, enzyme tethering, and cross-linking (via polymer backbone-to-polymer backbone tethers) in a single step.

FIG. 12C shows a schematic cartoon representation for electron shuttle installation, enzyme tethering, and cross-linking (via polymer backbone-to-polymer backbone tethers) in sequential steps.

FIGS. 13A-13G show various reactants and products for tethering electron shuttles to amino end groups of polymer pendant groups using various electron shuttles configured with tethering arms (e.g., spacer arms with a unreacted terminal linking group). FIG. 13A shows a cross-linked polymer with primary and quaternary amine-tethered pendants generated using monomers such as those shown in FIGS. 1A-1D that can be used as a reactant for tethering electron shuttles. FIG. 13B shows a ferrocene species (an electron shuttle) with a conjugated tethering arm comprising an epoxide as an unreacted terminal reactive group. FIG. 13C shows a polymer product from reacting the polymer of FIG. 13A with the ferrocene electron shuttle of FIG. 13B. FIG. 13D shows a generalized structure of a naphthoquinone electron shuttle with a conjugated tethering arm. FIG. 13E shows an electron shuttle-tethered polymer resulting from reacting the electron shuttle of FIG. 13D with the polymer of FIG. 13A. In FIGS. 13D and 13E, each R¹¹ is a linking group, wherein the R¹¹ moieties in FIG. 13D are one each of a unreacted terminal linking group and a reacted linking group, and R¹¹ moieties in FIG. 13E are both reacted linking groups, and R⁴ is a spacer arm. The sulfonamide (the —SO₂NR^{x}₂ subunit) bound to R¹¹ in FIGS. 13D and 13E can be replaced with sulfhydryl, ester, substituted aryl (such as fluorinated benzene ring), heterocycles, amides, alkylene-amide, or other functional group that is stable, preferably electron withdrawing, and serves as a bridge or connection to a linking group on a tethering agent, or may be absent. FIG. 13F shows a piperazine-containing naphthoquinone electron shuttle with a conjugated tethering arm. FIG. 13G depicts a naphthquinone-tethered polymer product resulting from reacting the electron shuttle of FIG. 13F with a pendant amine on a polymer. In FIGS. 13A-13G, each instance of m, n, o, p, q, and r independently represents a positive integer. In some versions, r can be 1-10.

FIG. 14A shows an exemplary structural representation for electron shuttle installation, enzyme tethering, and cross-linking (via polymer backbone-to-polymer backbone tethers) in a single step with polymers made with the monomers of FIGS. 3A and 3C and acrylamide.

FIG. 14B shows an exemplary structural representation for electron shuttle installation, enzyme tethering, and cross-linking (via polymer backbone-to-polymer backbone tethers) in a single step with polymers made with the monomer of FIG. 3D and acrylamide.

FIGS. 15A-15Q show various exemplary reactions for independently installing electron shuttles and cross-linking polymer chains post polymerization on an exemplary starting polymer chain using a variety of different orthogonal chemistries. FIG. 15A shows coupling of an exemplary 2-naphthoquinone electron shuttle to an exemplary linear polymer using NHS-ester based coupling. FIG. 15B shows the preparation the exemplary 2-naphthoquinone electron shuttles used in the NHS-ester based coupling of FIG. 15A and the carbodiimide COOH-amine coupling of FIG. 15L. FIG. 15C shows a first step in tetrazole photo-click cross-linking of the polymer product of FIG. 15A. FIG. 15D shows a second step in tetrazole photo-click cross-linking of the polymer product of FIG. 15A. FIG. 15E shows a first step of coupling of an exemplary ALO-containing 2-naphthoquinone electron shuttle or an exemplary DIMAC-containing 2-naphthoquinone electron shuttle to an exemplary linear polymer using Cu-free azide-cycloalkyne click chemistry. FIG. 15F shows a second step of coupling of an exemplary ALO-containing 2-naphthoquinone electron shuttle or an exemplary DIMAC-containing 2-naphthoquinone electron shuttle to an exemplary linear polymer using Cu-free azide-cycloalkyne click chemistry. FIG. 15G shows the preparation of the exemplary ALO-containing electron shuttle used in the coupling of FIG. 15F. FIG. 15H shows the preparation of the exemplary DIMAC-containing electron shuttle used in the coupling of FIG. 15F. FIG. 15I shows a first step in tetrazole photo-click cross-linking of the polymer product of FIG. 15F. FIG. 15J shows a second step in tetrazole photo-click cross-linking of the polymer product of FIG. 15F. FIG. 15K shows epoxide based cross-linking of the polymer product of FIG. 15F. FIG. 15L shows coupling of an exemplary 2-naphthoquinone electron shuttle to an exemplary linear polymer using carbodiimide COOH-amine coupling. See FIG. 15B for the preparation of the exemplary 2-naphthoquinone electron shuttle used in the coupling of FIG. 15L. FIG. 15M shows NHS-ester based cross-linking of the polymer product of FIG. 15L. FIG. 15N shows a first step in diazirine photochemical cross-linking of the polymer product of FIG. 15L. FIG. 15O shows a second step in diazirine photochemical cross-linking of the polymer product of FIG. 15L. FIG. 15P shows a first step in aryl azide photochemical cross-linking of the polymer product of FIG. 15L. FIG. 15Q shows a second step in aryl azide photochemical cross-linking of the polymer product of FIG. 15L.

FIGS. 16A and 16B show cartoon illustrations of detection system operation based on (FIG. 16A) direct hydrogen peroxide detection and (FIG. 16B) electron shuttle-based detection with tethered electron shuttles.

FIGS. 17A-17D shows cartoon illustrations of four exemplary electrochemical detection modes, including (FIG. 17A) direct hydrogen peroxide detection, (FIG. 17B) electron shuttle-based detection, (FIG. 17C) mediated hydrogen peroxide oxidation detection, (FIG. 17D), mediated hydrogen peroxide reduction detection. Depicted steps include: (1) glucose binding and enzyme-catalyzed oxidation resulting in the reduced form of enzyme; (2) oxidation of reduced form of enzyme by oxygen to form hydrogen peroxide; (3) diffusion of hydrogen peroxide; (4) electrochemical oxidation of hydrogen peroxide at the working electrode; (2B) oxidation of reduced form of enzyme by oxidized form of electron shuttle resulting in reduced form of electron shuttle; (3B) transport of electrons to electrode via tethered or dissolved electron shuttles; (4B) oxidation of reduced form of electron shuttles at working electrode; (4C) mediated oxidation of hydrogen peroxide; (5C) oxidation of reduced form of mediator at working electrode; (4D) mediated reduction of hydrogen peroxide; and (5D) reduction of oxidized form of mediator at working electrode.

FIGS. 18A-18C show exemplary redox mediators capable of undergoing redox reactions with hydrogen peroxide. FIG. 18A shows a cobalt-phthalocyanine redox mediator.

FIG. 18B shows a ferrocyanide redox mediator. FIG. 18C shows a Prussian blue redox mediator.

FIG. 19 shows a cartoon illustration of a dual detection mode system in which the operating potential is such that both hydrogen peroxide and the reduced form of the electron shuttle can be oxidized at the working electrode. This detection mode permits collecting electrons from enzymatic glucose oxidation in the presence or absence of oxygen and is therefore less susceptible to problems caused by variations in oxygen concentration. Depicted steps include: (1) glucose binding and enzyme-catalyzed oxidation resulting in the reduced form of enzyme; (2A) oxidation of reduced form of enzyme by oxidized form of electron shuttle resulting in reduced form of electron shuttle; (3A) transport of electrons to electrode via tethered or dissolved electron shuttles; (4A) oxidation of reduced form of electron shuttles at working electrode; (2B) oxidation of reduced form of enzyme by oxygen to form hydrogen peroxide; (3B) diffusion of hydrogen peroxide; and (4B) electrochemical oxidation of hydrogen peroxide at the working electrode.

FIG. 20. Sensor response data as a function of randomly ramped glucose concentration corresponding to a microneedle equipped with two high surface area Pt electrodes in contact with a redox hydrogel layer and glucose flux regulating polymer membrane top coating. Sensor operated in 2-electrode configuration i-t mode using +50 mV operating potential under ambient conditions with 100 mM PB buffer (pH 7.4) electrolyte.

FIG. 21. Sensor response data as a function of linearly ramped glucose concentration corresponding to a custom chip equipped with two small-cross-section, high-surface-area Pt electrodes in contact with a redox hydrogel layer and a glucose-flux-regulating, polymer-membrane top coating. Sensor operated in 2-electrode configuration i-t mode using +50 mV operating potential under ambient conditions with 100 mM PB buffer (pH 7.4) electrolyte.

FIG. 22. Low glucose concentration range sensor response data as a function of linearly ramped glucose concentration corresponding to a microneedle equipped with two high surface area Pt electrodes in contact with a redox hydrogel layer and glucose flux regulating polymer membrane top coating. Sensor operated in 2-electrode configuration i-t mode using +50 mV operating potential under ambient conditions with 100-mM PB buffer (pH 7.4) electrolyte. Inset: Corresponding calibration curve consisting of average steady state current values (averaged over the latter 100 seconds of each 200 sec sample interval) plotted as a function of glucose concentration (gray diamonds) and the corresponding linear fit (dashed line).

FIG. 23. Overlaid i-t traces corresponding to the response of a sensing electrode composed of a Pt wire coated with Gen. I cationic GOx hydrogel operating in hydrogen peroxide oxidation detection to various glucose concentrations. Sensor operated in 3-electrode configuration i-t mode with hydrogel coated Pt wire working electrode, Pt wire coil counter electrode and Ag/AgCl reference electrode operating at +600 mV under ambient conditions with 100 mM PB buffer (pH 7.4) electrolyte.

### DETAILED DESCRIPTION OF THE INVENTION

One aspect of the invention includes polymers. The polymers are preferably configured to form hydrogels that can be used, for example, in next generation enzymatic biosensing technologies.

Polymers of the invention encompass polymers that include subunits of Formula I:

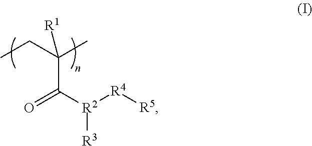

wherein:
R¹ in each instance is independently H, alkyl, cycloalkyl, aryl, heterocycle, heteroaryl, or a combination thereof;
R² in each instance is independently N or O;
R³ in each instance is independently:
   H; alkyl, cycloalkyl, aryl, heterocycle, heteroaryl, or a combination thereof; or a nitrogen protecting group when R² is N; or
   absent when R² is O;
R⁴ in each instance is a spacer arm;
R⁵ in each instance is Formula II or Formula III:

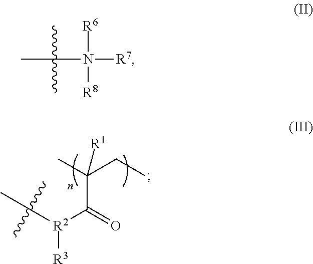

R⁶, R⁷, and R⁸ in each instance are independently H; alkyl, cycloalkyl, aryl, heterocycle, heteroaryl, or a combination thereof; a nitrogen protecting group; a tethered polypeptide; a tethered redox mediator; a linking arm; or a tethered subunit of Formula I, with the proviso that at least one of R⁶, R⁷, and R⁸ may be absent; and
n in each instance is independently a positive integer.
   When all three R groups are present in Formula II, there is a positive charge on the nitrogen.

The alkyls described herein include substituted or unsubstituted, linear or branched, saturated carbon groups. Exemplary alkyls include (C1-C18)alkyl, (C1-C12)alkyl, and (C1-C6)alkyl, such as (C1)alkyl (methyl), (C2)alkyl (ethyl), (C3)alkyl (propyl, including n-propyl and isopropyl), and (C4)alkyl (butyl, including isobutyl, sec-butyl, and ten-butyl).

The cycloalkyls described herein include substituted or unsubstituted saturated cyclic alkyl groups. Exemplary cycloalkyls include (C1-C18)cycloalkyl, (C1-C12)cycloalkyl, (C1-C7)cycloalkyl, or (C1-C7)cycloalkyl, such as cyclopropyl, cyclobutyl, cyclopentyl, cyclohexyl and cycloheptyl.

The aryls described herein include substituted or unsubstituted functional groups containing one or more aromatic rings. The aryls can be monocyclic, bicyclic, tricyclic, etc. Exemplary aryls include phenyl, benzyl, naphthyl, anthracenyl, thienyl, and indolyl.

The heterocycles and heteroaryls described herein include cycloalkyls and aryls in which one or more carbons are replaced with an atom other than carbon (e.g., sulfur, oxygen, or nitrogen).

The functional groups described herein, including the alkyls, cycloakyls, aryls, heterocycles, and heteroaryls, etc., can be substituted with one or more substituents. Unless a functional group is explicitly listed as “unsubstituted,” the broad recitation of any functional group encompasses both substituted and unsubstituted versions, where possible. Exemplary substituents include alkyl, alkynyl, cycloalkynyl, cycloalkyl, hydroxyl, halo (e.g., Cl, Br, F), haloalkyl, aryl, aldehyde, halogen-substituted aryl (including aryl groups substituted multiple times with the same or different halogen), nitro-substituted aryl, heterocycle, heteroaryl, hydroxyl, halo, haloalkyl, amino, an amino protected with a nitrogen protecting group, nitro, pyridine, ester, amide, azide, sulfate, sulfite, sulfoalkyl, sulfhydryl, sulfonamide, thiazole, nitro, cyano, alkoxy, carboxy, aldehyde, a saturated or unsaturated cycloalkyl, a saturated or unsaturated heterocycle, bridged saturated and unsaturated cylcoalkyl, fused aromatic, aromatic heterocycle, an N-hydroxysuccinimide ester-reactive group, an amine-reactive group, a sulfhydryl-reactive group, a tethered polypeptide, a tethered redox mediator, a linking arm, a tethered subunit, and combinations thereof.

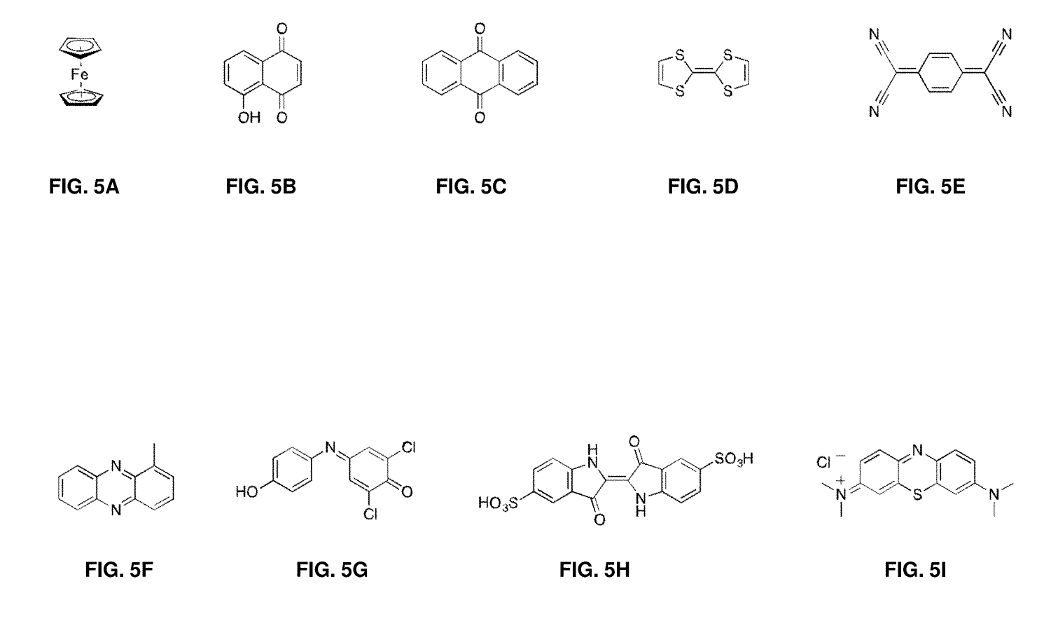

The nitrogen protecting groups described herein include benzyl ethers, silyl ethers, esters including sulfonic acid esters, carbonates, sulfates, and sulfonates, among others. For example, suitable nitrogen protecting groups include substituted methyl ethers; substituted ethyl ethers; p-chlorophenyl, p-methoxyphenyl, 2,4-dinitrophenyl, benzyl; substituted benzyl ethers (p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2- and 4-picolyl, diphenylmethyl, 5-dibenzosuberyl, triphenylmethyl, p-methoxyphenyl-diphenylmethyl, di(p-methoxyphenyl)phenylmethyl, tri(p-methoxyphenyl)methyl, 1,3-benzodithiolan-2-yl, benzisothiazolyl S,S-dioxido); silyl ethers (silyloxy groups) (trimethylsilyl, triethylsilyl, triisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, t-butylmethoxy-phenylsilyl); esters (formate, benzoylformate, acetate, choroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), pivaloate, adamantoate, crotonate, 4-methoxycrotonate, benzoate, p-phenylbenzoate, 2,4,6-trimethylbenzoate (mesitoate)); carbonates (methyl, 9-fluorenylmethyl, ethyl, 2,2,2-trichloroethyl, 2-(trimethylsilyl)ethyl, 2-(phenylsulfonyl)ethyl, 2-(triphenylphosphonio)ethyl, isobutyl, vinyl, allyl, p-nitrophenyl, benzyl, p-methoxybenzyl, 3,4-dimethoxybenzyl, o-nitrobenzyl, p-nitrobenzyl, S-benzyl thiocarbonate, 4-ethoxy-1-naphthyl, methyl dithiocarbonate); groups with assisted cleavage (2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate, 4-(methylthiomethoxy)butyrate, miscellaneous esters (2,6-dichloro-4-methylphenoxyacetate, 2,6-dichloro-4-(1,1,3,3 -tetramethylbutyl)phenoxyacetate, 2,4-bis(1,1-dimethylpropyl)phenoxyacetate, chlorodiphenylacetate, isobutyrate, monosuccinate, (E)-2-methyl-2-butenoate (tigloate), o-(methoxycarbonyl)benzoate, p-poly-benzoate, α-naphthoate, nitrate, alkyl N,N,N′,N′-tetramethyl-phosphorodiamidate, n-phenylcarbamate, borate, 2,4-dinitrophenylsulfenate); or sulfonates (methanesulfonate (mesylate), benzenesulfonate, benzylsulfonate, tosylate, or triflate). Polymers in which R⁵ is Formula III can be made by including diacrylate or diacrylamide monomers, such as those shown in FIGS. 5A and 5B, in the polymerization process.

The subscript n used herein for Formula I and Formula III refers independently in each instance to any positive integer. Examples include positive integers within a range of 1-50,000 inclusive or more, such as 1-25,000, 1-10,000, 1-5,000, 1-2,500, 1-1,000, 1-500, 1-250, 1-100, 1-50, 1-40, 1-30, 1-20, 1-10, 1-5, or 1-2.

The moiety of Formula II can be positively charged or neutral, depending on whether R⁸ is present or absent. The polymers described herein encompass both salt and non-salt forms.

The tethered polypeptide can include any polypeptide. The polypeptide can have any number of amino acid residues, such as from 2 to about 10, to about 50, to about 100, to about 150, to about 300, to about 1000, to about 2,000, to about 3,000, to about 4,000, to about 4,500 or more residues. The polypeptide can have any function. For example, the polypeptide can have a binding function, a structural function, an enzymatic function, or any other function.

In some versions, the polypeptide is an enzyme. Exemplary types of enzymes include transferases, hydrolases, lyases, isomerases, and ligases. Transferases are enzymes that transfer functional groups (e.g., amino or phosphate groups). Hydrolases are enzymes that transfer water or catalyze the hydrolysis of a substrate. Lyases are enzymes that add or remove the elements of water, ammonia, or carbon dioxide to or from double bonds. Ligases join two molecules.

For electrochemical applications, the enzyme is preferably an oxidoreductase. Oxidoreductases are enzymes that catalyze the transfer of electrons from one molecule, the reductant, also called the electron donor, to another, the oxidant, also called the electron acceptor. Oxidoreductases usually utilize NADP, NAD+, FAD/FADH₂ as cofactors. Exemplary oxidoreductases include those falling under EC 1.1, which include oxidoreductases that act on the CH—OH group of donors (alcohol oxidoreductases); EC 1.2, which include oxidoreductases that act on the aldehyde or oxo group of donors; EC 1.3, which include oxidoreductases that act on the CH—CH group of donors (CH—CH oxidoreductases); EC 1.4, which include oxidoreductases that act on the CH—NH₂ group of donors (amino acid oxidoreductases, monoamine oxidase); EC 1.5, which include oxidoreductases that act on CH—NH group of donors; EC 1.6, which include oxidoreductases that act on NADH or NADPH; EC 1.7, which include oxidoreductases that act on other nitrogenous compounds as donors; EC 1.8, which include oxidoreductases that act on a sulfur group of donors; EC 1.9, which include oxidoreductases that act on a heme group of donors; EC 1.10, which include oxidoreductases that act on diphenols and related substances as donors; EC 1.11, which include oxidoreductases that act on peroxide as an acceptor (peroxidases); EC 1.12, which include oxidoreductases that act on hydrogen as donors; EC 1.13, which include oxidoreductases that act on single donors with incorporation of molecular oxygen (oxygenases); EC 1.14, which include oxidoreductases that act on paired donors with incorporation of molecular oxygen; EC 1.15, which include oxidoreductases that act on superoxide radicals as acceptors; EC 1.16, which include oxidoreductases that oxidize metal ions; EC 1.17, which include oxidoreductases that act on CH or CH₂ groups; EC 1.18, which include oxidoreductases that act on iron-sulfur proteins as donors; EC 1.19, which include oxidoreductases that act on reduced flavodoxin as a donor; EC 1.20, which include oxidoreductases that act on phosphorus or arsenic in donors; EC 1.21, which include oxidoreductases that act on X—H and Y—H to form an X—Y bond; EC 1.97, which include other oxidoreductases.

For the detection of glucose, a preferred oxidoreductase is glucose oxidase (GOx). also known as notatin (EC number 1.1.3.4). Glucose oxidase is an oxido-reductase that catalyzes the oxidation of glucose to hydrogen peroxide and D-glucono-δ-lactone.

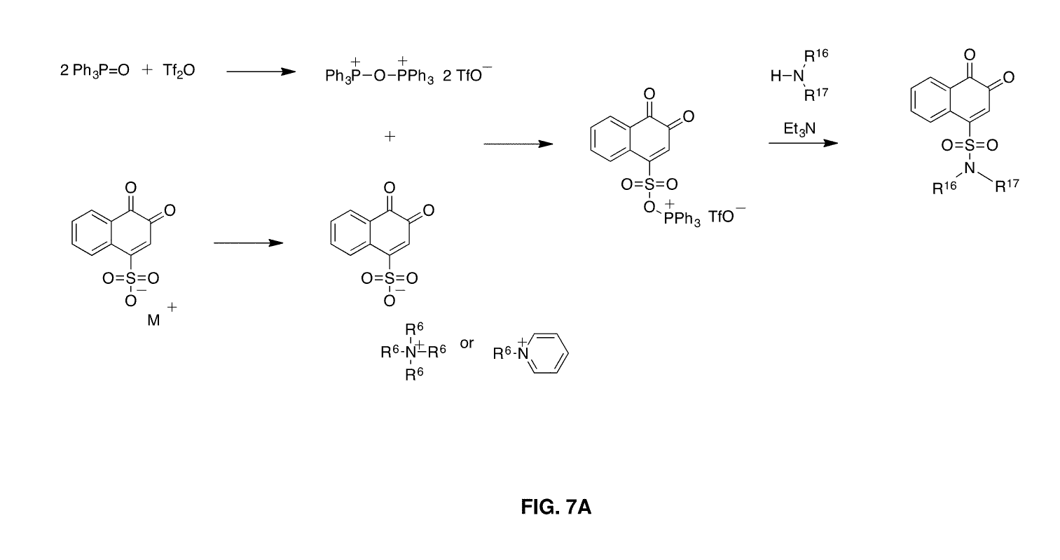

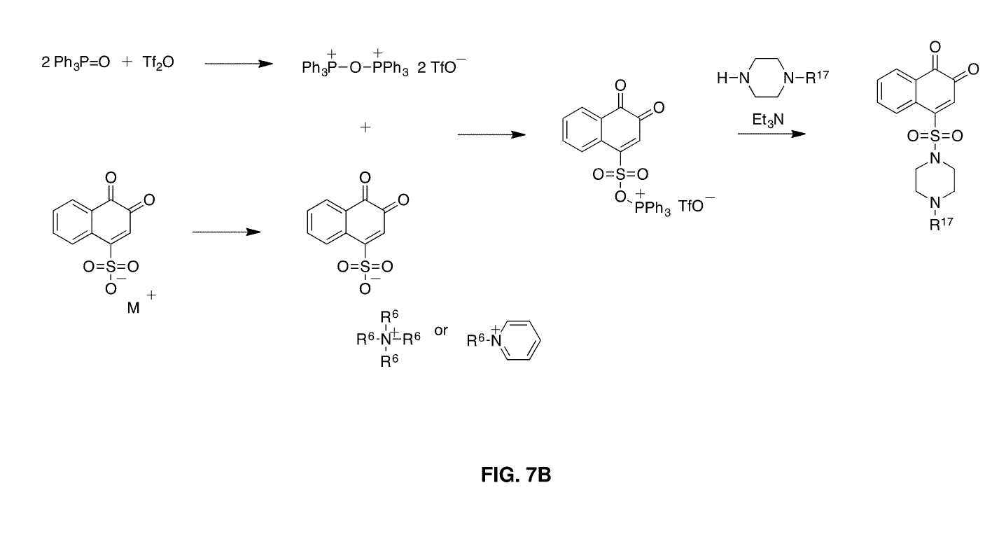

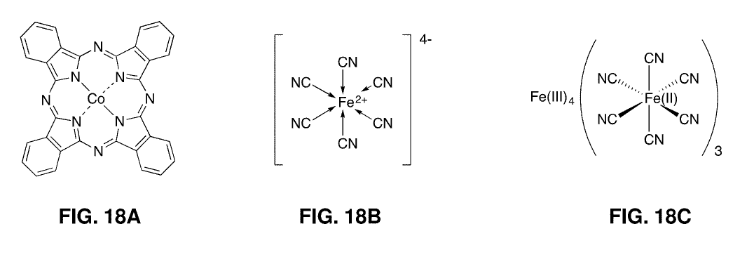

The tethered redox mediator comprises any compound or moiety capable of undergoing a reversible oxidation-reduction (redox) reaction, e.g., a reaction that involves a transfer of one or more electrons between chemical species. A large number of redox mediators are known in the art. Examples redox mediators include those provided in FIGS. 5A-5I, 6, 7A, 7B, 8, and 18A-18C and analogs thereof. Some redox mediators oxidize reduced molecules, such as a reduced oxidoreductase enzyme, and transfer the electrons to a medium, other molecules, or an electrode. Such redox mediators are referred to herein as “electron shuttles.” Examples of electron shuttles are those provided in FIGS. 5A-5I, 6, 7A, 7B, and 8, and analogs thereof. Some redox mediators undergo redox reactions with hydrogen peroxide and transfer the electrons to or from a medium, other molecules, or an electrode. Examples of such redox mediators are provided in FIGS. 18A-18C. Electron shuttles can be identified by exhibiting transfer of electrons, e.g. electron transfer from reduced form of glucose oxidase to the oxidized form of an electron shuttle, in an inert atmosphere, e.g., in the absence of oxygen, such that hydrogen peroxide cannot be generated.

In some versions, the electron shuttle comprises a compound of Formula VI:

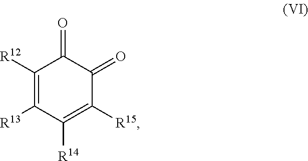

wherein:
R¹² and R¹³ are each independently H, alkyl, cycloalkyl, aryl, heterocycle, heteroaryl, or a combination thereof; or are fused in an aromatic or non-aromatic ring;
R¹⁴ is an electron withdrawing group, such as a sulfonate, a sulfonamide, an ammonium, a quaternary ammonium, a fluoroaklyl, a perfluoroalkyl, a nitro, a cyano, or a combination thereof;
R¹⁵ is H, alkyl, cycloalkyl, aryl, heterocycle, heteroaryl, or a combination thereof;
and the compound of Formula VI is optionally tethered to a monomer, polymer, or polymer subunit via any one or more of R¹², R¹³, R¹⁴, R¹⁵, and any atom in the aromatic or non-aromatic ring formed by R¹² and R¹³.

In some versions, R¹² and R¹³ are fused in a C6 aromatic ring, wherein the C6 aromatic ring includes R¹², R¹³, the carbons in Formula VI to which R¹² and R¹³ are bound, and two additional carbons.

In some versions, R¹⁴ in the electron shuttle of Formula VI is Formula VII or Formula VIII:

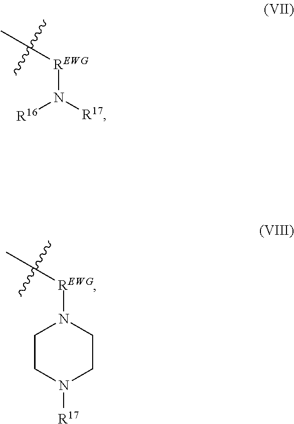

wherein:
R^{EWG} is an electron withdrawing group;
R¹⁶ is H, alkyl, alkyl, cycloalkyl, aryl, heterocycle, heteroaryl, perfluoroalkyl, or a combination thereof; and

R¹⁷ is H, alkyl, cycloalkyl, aryl, heterocycle, heteroaryl, or a combination thereof; a tethered polymer; a tethered monomer; or a linking arm. R¹⁷ can be tethered to a polymer monomer or a polymer via any one of R⁶, R⁷, and R⁸ as described for the polymers and monomers herein.

Analogs of the redox mediators explicitly provided herein include isomers and substituted versions of the redox mediators. For example, analogs of naphthoquinone include unsubstituted 1,4-naphthoquinone, unsubstituted 1,2-naphthoquinone, unsubstituted 2,3-naphthoquinone, unsubstituted 2,6-naphthoquinone, and substituted versions thereof, including 2-hydroxy-1,4-naphthoquinone, 5-hydroxy-1,4-naphthoquinone, 6-hydroxy-1,4-naphthoquinone, 3-hydroxy-1,2-naphthoquinone, 4-hydroxy-1,2-naphthoquinone, 5-hydroxy-1,2-naphthoquinone, 6-hydroxy-1,2-naphthoquinone, 7-hydroxy-1,2-naphthoquinone, 8-hydroxy-1,2-naphthoquinone, 1-hydroxy-2,3-naphthoquinone, 5-hydroxy-2,3-naphthoquinone, 6-hydroxy-2,3-naphthoquinone, 1-hydroxy-2,6-naphthoquinone, 3-hydroxy-2,6-naphthoquinone, 4-hydroxy-2,6-naphthoquinone, and (poly)hydroxynaphthoquinones, including dihydroxynaphthoquinone, trihydroxynaphthoquinone, tetrahydroxynaphthoquinone, pentahydroxynaphthoquinone, and hexahydroxynaphthoquinone. Other redox mediators include sulfonate- or sulfonamide-substituted redox mediators, such as 1,2-naphthoquinone-4-sulfonates, 1,2-naphthoquinone-4-sulfonamides, and others. Substituted versions of naphthoquinone isomers with other substituents and analogs of the other redox mediators explicitly provided herein are well known in the art.

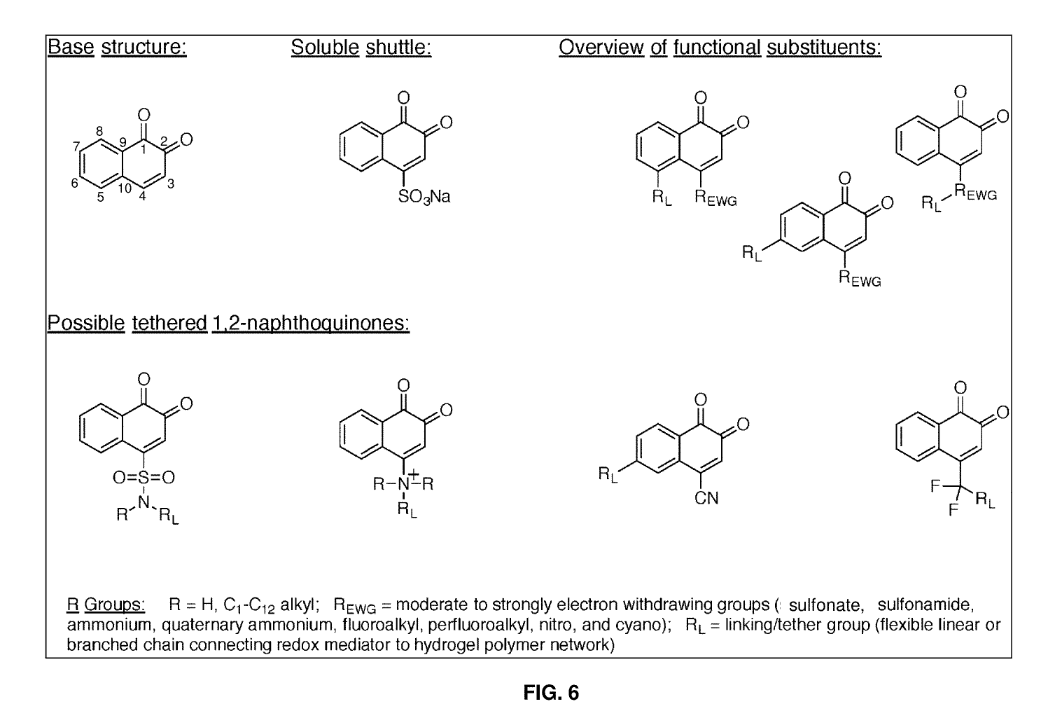

In some versions of the invention, tethered redox mediator comprises a 1,2-naphthoquinone. See FIGS. 6-8. The general term “1,2-naphthoquinone” encompasses unsubstituted 1,2-naphthoquinone (see “base structure” in FIG. 6) and any substituted versions thereof, such as 3-hydroxy-1,2-naphthoquinone, 4-hydroxy-1,2-naphthoquinone, 5-hydroxy-1,2-naphthoquinone, 6-hydroxy-1,2-naphthoquinone, 7-hydroxy-1,2-naphthoquinone, 8-hydroxy-1,2-naphthoquinone, and others. Preferred 1,2-naphthoquinones of the invention comprise an electron withdrawing group at the 4-carbon of the 1,2-naphthoquinone. (For the carbon numbering of 1,2-napthoquinones, see the “base structure” in FIG. 6) Exemplary electron withdrawing groups include sulfonate, sulfonamide, ammonium, quaternary ammonium, fluoroaklyl, perfluoroalkyl, nitro, and cyano groups. Others known the art are acceptable. The 1,2-naphthoquinones can be tethered to the polymer via the 3-carbon, the 4-carbon, the 5-carbon, the 6-carbon, the 7-carbon, or the 8-carbon of the 1,2-naphthoquinone. If tethered via the 4-carbon, the electron withdrawing group can serve as an intermediary between the tether and the 4-carbon.

The tethered subunits include tethered subunits having a structure of Formula I. Some tethered subunits having a structure of Formula I can be tethered via the nitrogen of Formula II at any one of R⁶, R⁷, or R⁸ of the tethered subunit. Some tethered subunits having a structure of Formula I can be tethered via a nitrogen (derived from an amino) or sulfur (derived from a sulfhydryl) at R⁹ of Formula IV. Accordingly, some polymers of the invention include subunits of Formula I tethered to each other via R⁶, R⁷, or R⁸ of the respective tethered subunits. Some polymers of the invention include subunits of Formula I tethered to each other via a nitrogen (derived from an amino) or sulfur (derived from a sulfhydryl) at R⁹ of Formula IV in the respective tethered subunits. Some polymers of the invention include subunits of Formula I tethered via R⁶, R⁷, or R⁸ to a nitrogen (derived from an amino) or a sulfur (derived from a sulfhydryl) at R⁹ of Formula IV in corresponding tethered subunits. The corresponding tethered subunits can be from the same individual polymer backbone, thereby forming an intra-backbone crosslink, or can be from separate polymer backbones, thereby forming inter-backbone crosslinks. The tethers thereby provide effective crosslinks between one or more individual polymer backbones in the polymer.

The term “spacer arm” refers to any linear, branched, and/or cyclic moiety connecting two other moieties. The spacer arms in some aspects are preferably flexible. The spacer arms can include substituted or unsubstituted C1-C25 alkylenes. Exemplary spacer arms include one or more instances of a moiety selected from the group consisting of —(CH₂)ₘ—, —(CH₂)ₘ—O—(CH₂)ₘ—, —(CH₂)ₘ—(NR¹⁸R¹⁹)—(CH₂)ₘ—, and combinations thereof. R¹⁸ and R¹⁹ in a given spacer arm can independently be H; alkyl, cycloalkyl, aryl, heterocycle, heteroaryl, or a combination thereof; or a nitrogen protecting group, with the proviso that at least one of R¹⁸ and R¹⁹ may be absent. In some versions, the spacer arm can include moieties such as (—(CH₂)₂—O)ₘ—(CH₂)₂—. The subscript m in any given spacer arm is a positive integer. Examples include positive integers from 1 to 20, inclusive, such as 1, 2, 3, 4, 5, 6, 7, 8, 9, 10, 11, 12, 13, 14, 15, 16, 17, 18, 19, or 20, or any ranges therebetween.

Tethers, tethering agents, and tethering arms used to tether two components to each other preferably include at least one linking group. The term “linking group” refers to a moiety comprising a functional group capable of covalently reacting with (or reacted with) a functional group on another moiety. Moieties that are capable of interacting with each other are referred to herein as “corresponding linking groups.”

Preferred tethers, tethering agents, and tethering arms include one or more internal spacer arms, two or more terminal linking groups, and, optionally, one or more internal linking groups. The spacer arms can include any spacer arm as described herein. The terminal linking groups include functional groups capable of reacting with (or reacted with) linking groups on the components that are to be (or are) linked. The internal linking groups are pairs of linking groups reacted within the tethers, tethering agents, and tethering arms themselves and can link two or more spacer arms to each other.

The tethers, tethering agents, and tethering arms are identified according to whether particular terminal linking groups are reacted, and thus conjugated with a corresponding linking group, or unreacted, and thus not yet conjugated with a corresponding linking group. Tethers, for example, include at least two reacted linking groups at each end of the tether; tethering agents include at least two unreacted terminal linking groups; and tethering arms include at least one reacted linking group at one end of the tethering arm and at least one unreacted terminal linking group. Thus, tethers actively link two components to each other via the reacted linking groups, tethering agents have the ability to link two components to each other via the unreacted terminal linking groups, and tethering arms are linked to a first component via the reacted linking group and have the ability to link the first component to a second component via the unreacted terminal linking group.

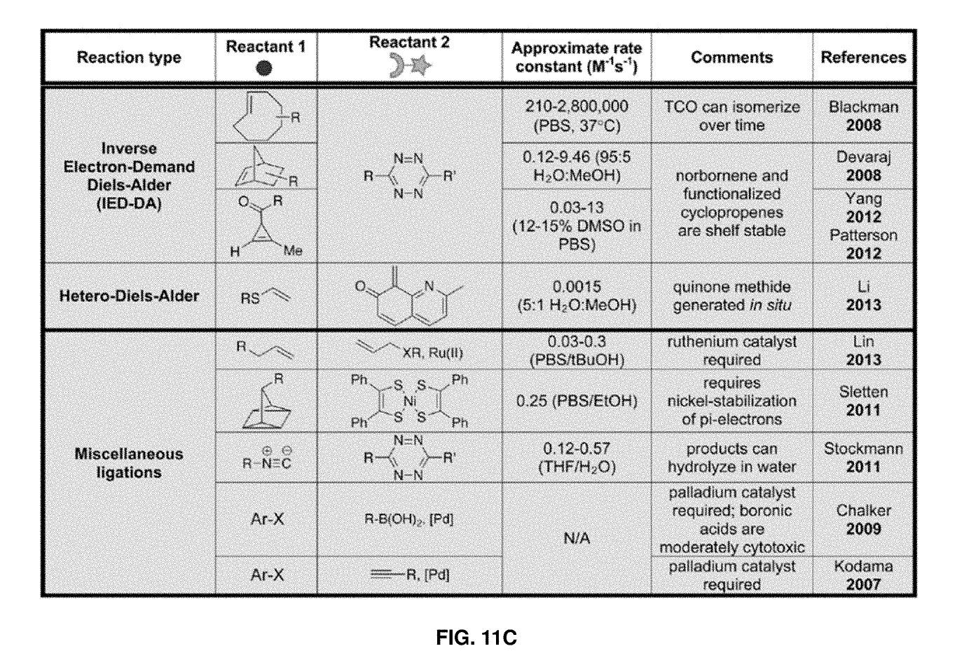

Exemplary corresponding linking groups include those shown in FIGS. 11A-11C. Components to be tethered can originally contain these groups or can be modified to contain them.

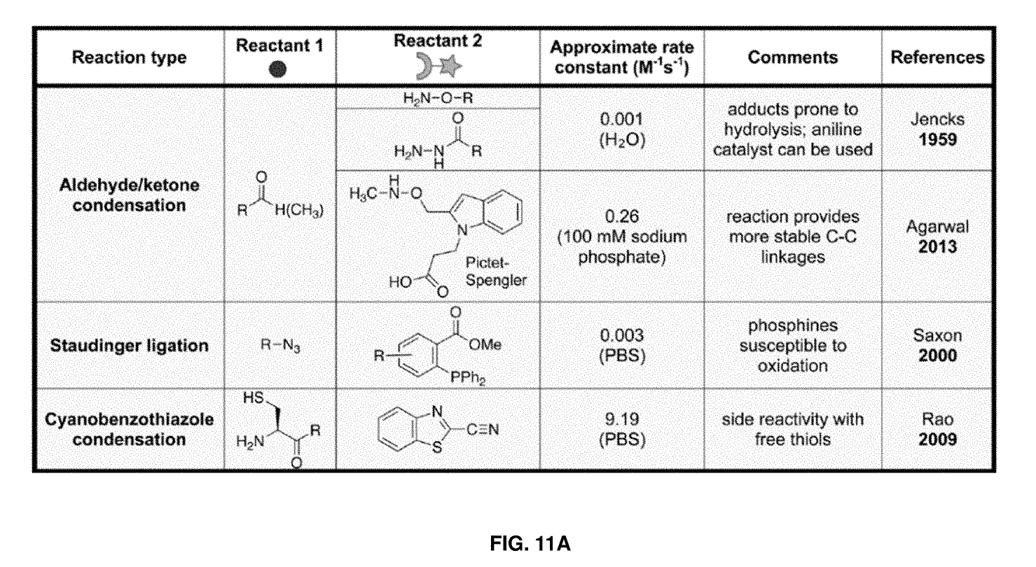

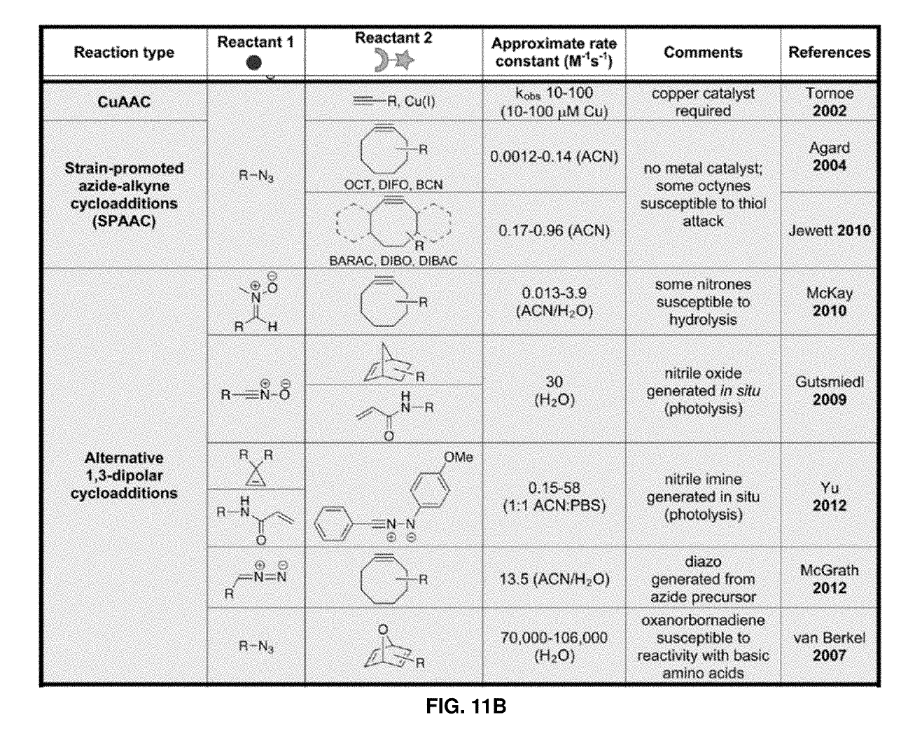

In some versions, at least one linking group on the tether, tethering agent, or tethering arm includes an amine-reactive functional group. The amine-reactive functional group can be a primary amine-reactive functional group. Amines can be included, for example, in the polymers at R⁵, at the N-terminus of polypeptide chains, and in the side-chain of lysine (Lys, K) amino acid residues. There are numerous synthetic chemical groups that will form chemical bonds with primary amines. These include isothiocyanates, isocyanates, acyl azides, N-hydroxysuccinimide (NHS) esters, sulfonyl chlorides, aldehydes, glyoxals, epoxides, oxiranes, carbonates, alkyl halides, aryl halides, imidoesters, carbodiimides, anhydrides, and fluorophenyl esters. Exemplary structures of some of these groups are shown in FIG. 10. Most of these conjugate to amines by either acylation or alkylation. In the case of alkylation, the carbon bound to the nitrogen is considered a reacted linking group (by virtue of an alkyl halide, for example, reacting with the nitrogen). Formaldehyde and glutaraldehyde are aggressive carbonyl (—CHO) reagents that condense amines via amine-carbonyl condensation reactions, Mannich reactions and/or reductive amination. NHS esters and imidoesters are common amine-specific functional groups that are incorporated into reagents for protein crosslinking and labeling.

In some versions of the invention, the tether, tethering agent, or tethering arm can include a sulfhydryl (thiol)-reactive functional group as a linking group. Sulfhydryls can be included, for example, in the side-chain of cysteine (Cys, C) amino acid residues. Exemplary sulfhydryl-reactive functional groups include haloacetyl (iodoacetyl, bromoacetyl, etc.), maleimide, and pyridyldithiol groups.

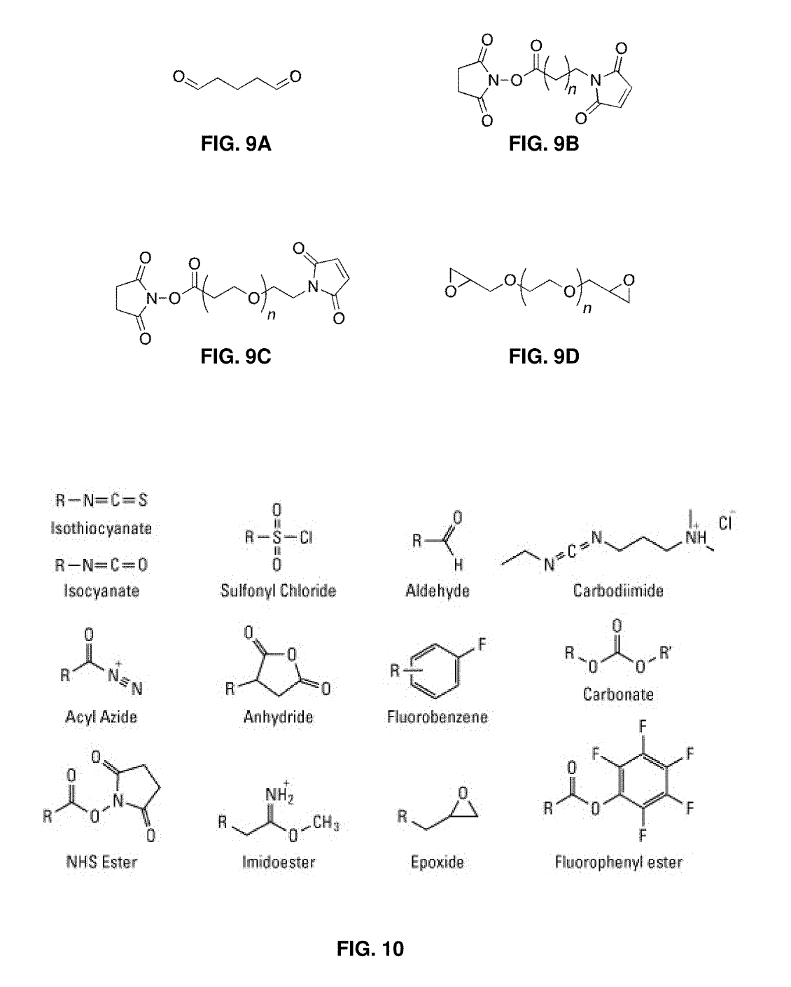

Exemplary tethering agents are shown in FIGS. 9A-9D. Various other suitable tethering agents or components thereof include any of the linkers or functional groups provided in the Thermo Scientific Crosslinking Technical Handbook (tools.thermofisher.com/ontent/sfs/brochures/1602163-Crosslinking-Reagents-Handbook.pdf), which is incorporated herein by reference.

The polymer in some versions can take the form of cross-linked polymer networks that include individual polymer backbones cross-linked to each other. The individual polymer backbones include the substituted alkylene chains (and terminal end groups) resulting from the polymerization of vinyl (ethenyl) groups in acrylic monomers. The cross-links can take the form of tethers between individual polymer backbones. The tethers can be formed post-polymerization via orthogonal chemistries on the polymer backbone by way of the pendant groups bearing reactive functionality. Suitable tethering agents used to form the tethers include those described above, particularly those having amine-reactive functional groups. Cross-linked polymers comprising tethers between individual polymer backbones include polymers that include subunits of Formula I wherein R⁵ is Formula II, wherein R⁸ a tethered subunit having a structure of Formula I wherein R⁵ in the tethered subunit has a structure of Formula II and is tethered at R⁸ of the tethered subunit.

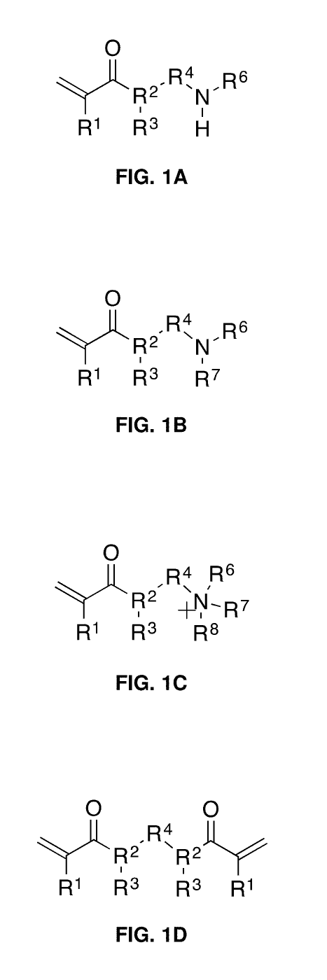

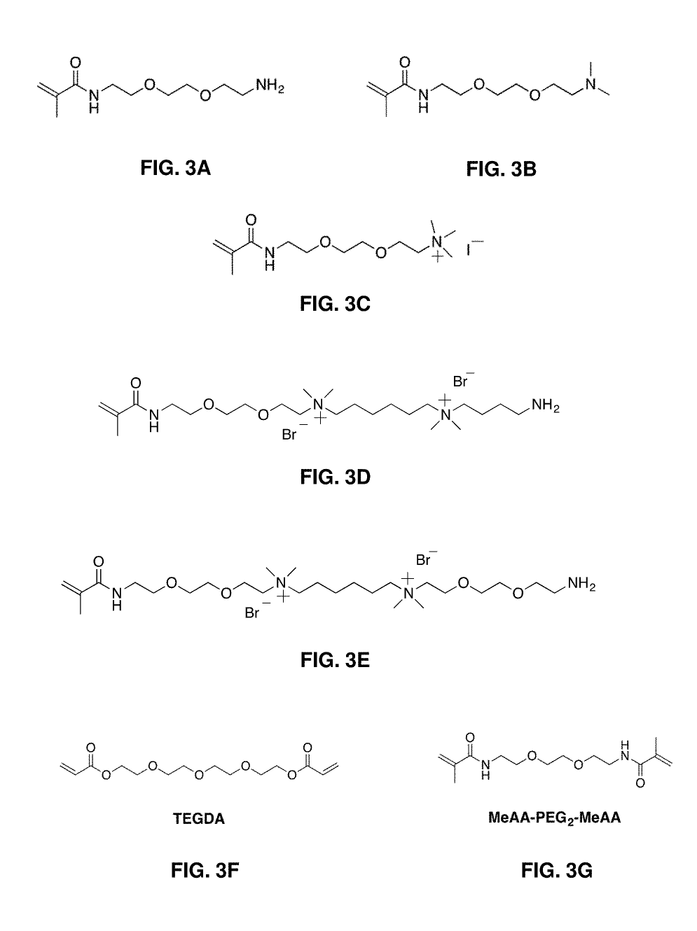

The cross-links can also or alternatively take the form of cross-linkers polymerized into the individual polymer backbones. Such cross-linkers preferably include an internal spacer arm and two or more terminal vinyl (ethenyl) groups. Exemplary cross-linkers are shown in FIGS. 1D, 3F, and 3G. Other exemplary cross-linkers include polyethylene glycol dimethacrylates, 1,6-hexanediol diacrylate, 1,6-hexanediol dimethacrylate, 1,9-nonanediol dimethacrylate, 1,4-butanediol dimethacrylate, 1,3-butanediol dimethacrylate, 1,10-decanediol dimethacrylate, diurethane dimethacrylate, 1,4-butanediol diacrylate, ethylene glycol diacrylate, 1,5-pentanediol dimethacrylate, 1,4-phenylene diacrylate, allyl methacrylate, 2,2-bis[4-(2-hydroxy-3-methacryloxypropoxy)phenyl]propane, tricyclodecane dimethanol diacrylate, tetraethylene glycol diacrylate, bis(2-methacryloxyethyl) phosphate, ethylene glycol dimethacrylate, diethylene glycol diacrylate, diethylene glycol dimethacrylate, triethylene glycol diacrylate, triethylene glycol dimethacrylate, tetraethylene glycol dimethacrylate, poly(ethylene glycol) diglycidyl ether, polyethylene glycol diacrylate, polyethylene glycol dimethacrylate, polyethylene glycol diacrylate, triethylene glycol dimethacrylate, and N,N-diallylacrylamide, among others. Cross-linked polymers comprising cross-linkers polymerized into the individual polymer backbones include polymers that include subunits of Formula I wherein R⁵ is Formula III.

In some versions, R⁵ in at least one subunit of the polymer is Formula II, and at least one of R⁶, R⁷, and R⁸ in Formula II is Formula IV:

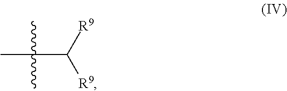

wherein R⁹ in each instance is independently hydrogen, alkyl, alkynyl, cycloalkyl, aryl, halogen-substituted aryl (including aryl groups substituted multiple times with the same or different halogen), nitro-substituted aryl, heterocycle, heteroaryl, hydroxyl, halo, haloalkyl, amino, an amino protected with a nitrogen protecting group, pyridine, ester, amide, azide, sulfate, sulfite, sulfoalkyl, sulfhydryl, sulfonamide, thiazole, nitro, cyano, alkoxy, carboxy, aldehyde, a saturated or unsaturated cycloalkyl, a saturated or unsaturated heterocycle, bridged saturated and unsaturated cylcoalkyl, fused aromatic, aromatic heterocycle, an N-hydroxysuccinimide ester-reactive group, an amine-reactive group, a sulfhydryl-reactive group, a tethered polypeptide, a tethered redox mediator, a linking arm, a tethered subunit of Formula I, or a combination thereof.

The backbone or cross-linked backbones in the polymer can include homopolymer backbones and/or copolymer backbones. Copolymer backbones can include random co-polymers and/or block co-polymers. Each backbone in the polymer can have from 1 to 50,000 subunits inclusive, such as from 1 to 25,000 subunits, 1 to 10,000 subunits, 1 to 5,000 subunits, 1 to 2,500 subunits, 1 to 1,000 subunits, 1 to 500 subunits, 1 to 250 subunits, 1 to 100 subunits, 1 to 50 subunits, 1 to 40 subunits, 1 to 30 subunits, 1 to 20 subunits, 1 to 10 subunits, 1 to 5 subunits, or 1 to 2 subunits.

In addition to subunits of Formula I, the polymers of the invention can also include subunits of Formula V:

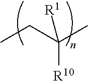

wherein, in each instance of Formula V, R₁ is H, alkyl, cycloalkyl, aryl, heterocycle, heteroaryl, or a combination thereof; n is a positive integer; and R¹⁰ is carboxyl or carboxamido. Such subunits can result from polymerizing acrylic monomers such as acrylamide, methacrylamide, acrylate, methacrylate, and analogs thereof and can be polymerized along with the acrylic monomer building blocks giving rise to the subunits Formula I.

The polymer can include the subunits of Formula I and Formula V in any relative proportion. Exemplary proportions instance ratios of Formula I and Formula V of from about 1:1000 (Formula I:Formula V) to about 1000:1 (Formula I:Formula V), from about 1:500 (Formula I:Formula V) to about 500:1 (Formula I:Formula V), from about 1:100 (Formula I:Formula V) to about 100:1 (Formula I:Formula V), from about 1:1000 (Formula I:Formula V) to about 1000:1 (Formula I:Formula V), from about 1:1000 (Formula I:Formula V) to about 2:1 (Formula I:Formula V), from about 1:500 (Formula I:Formula V) to about 1:1 (Formula I:Formula V), from about 1:100 (Formula I:Formula V) to about 1:1 (Formula I:Formula V), from about 1:50 (Formula I:Formula V) to about 1:1 (Formula I:Formula V), or from about 1:10 (Formula I:Formula V) to about 1:1 (Formula I:Formula V), wherein each instance of Formula I or Formula V is a structure of Formula I or Formula V with n=1. Each instance of Formula I and Formula V is counted separately regardless of whether or not the instances of Formula I or Formula V can be grouped as contiguous blocks (i.e., structures of Formula I or Formula V with n>1). For example, a polymer consisting of a subunit of Formula I with n=5 sandwiched between two subunits of Formula V, each with n=10, would have 5 instances of Formula I and 20 instances of Formula V and an instance ratio of 1:4 (Formula I:Formula V).

The polymers of the invention can form hydrogels when sufficiently interlinked or interconnected and dispersed in water. Accordingly, the hydrogels preferably include at least a polymer of the invention and water. In addition to the polymer and water, the hydrogel can include untethered redox mediators (including electron shuttles and those that oxidize/reduce hydrogen peroxide), salts, electrolytes, buffers, and other reagents or compounds dissolved or dispersed within the hydrogel. The untethered components of the hydrogel are not tethered to the polymer and can diffuse freely therein.

The hydrogel can be included in an electrochemical cell. The electrochemical cell has at least a counter electrode and a working electrode. The hydrogel composition contacts, at the minimum, the working electrode. An example of a suitable electrochemical cell includes a standard, three-electrode configuration that includes a working electrode, a counter electrode, and a reference electrode such that all electrodes, working and counter electrodes, or only the working electrode is equipped (in contact) with the hydrogel. Other electrochemical cells can be used, including those with fewer electrodes such as a two-electrode electrochemical cell, which includes a counter electrode and a working electrode. Working and counter electrode composition can include gold, platinum, or conductive carbon, among other materials. The reference electrode can include silver, among other materials. A preferred working and counter electrode composition is nanostructured platinum. Some versions employ a 2-electrode system with an Ag/AgCl counter electrode and a Pt working electrode.

The electrochemical cells can be used to detect and/or determine the concentration of an analyte in a sample. The sample can include a bodily fluid. The bodily fluid can include interstitial fluid, intravascular fluid, lymphatic fluid, or transcellular fluid. Particular examples of bodily fluids include amniotic fluid, aqueous humour, vitreous humour, bile, blood, blood plasma, blood serum, breast milk, cerebrospinal fluid, cerumen (earwax), chyle, chime, endolymph, perilymph, exudates, feces, diarrhea, female ejaculate, gastric acid, gastric juice, lymph, mucus (including nasal drainage and phlegm), pericardial fluid, peritoneal fluid, pleural fluid, pus, rheum, saliva, sebum (skin oil), serous fluid, semen, smegma, sputum, synovial fluid, sweat, tears, urine, vaginal secretion, and vomit. The analyte can include sugars, lipids, proteins, and small molecules, among others. The analyte is preferably one that is capable of being oxidized, such that electrons resulting from its oxidation can be detected and quantitated. A preferred analyte is glucose.

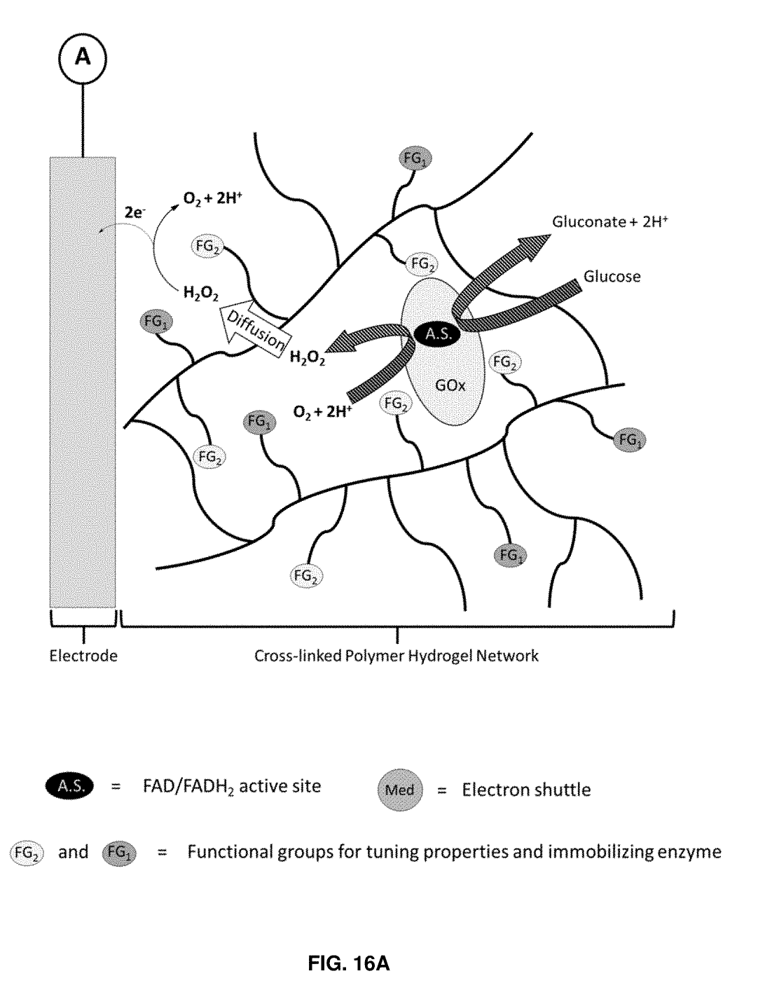

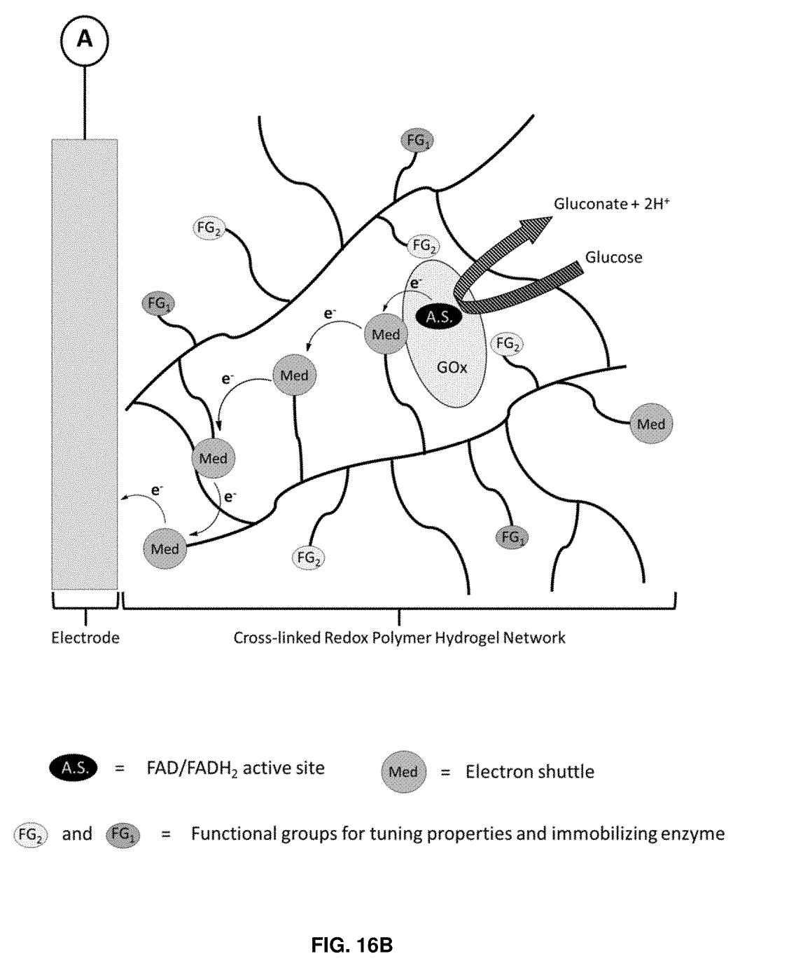

The electrochemical cells can be employed in a number of detection modes. These detection modes include direct hydrogen peroxide detection, electron shuttle-based detection, mediated hydrogen peroxide oxidation detection, mediated hydrogen peroxide reduction detection, and hybrids thereof. See, e.g., FIGS. 16A-16B, 17A-17D, and 19 and the following examples, which explain these detection modes in further detail.

Another aspect of the invention includes systems for detecting analytes. The systems can include any component described herein in any combination. Such components include polymers, enzymes, redox mediators, tethers, tethering arms, electrodes, etc. A subset of the components, such as the polymers, enzymes, redox mediators, tethers, etc., can be provided in the form of a hydrogel.

Another aspect of the invention includes methods of detecting analytes. The methods comprise contacting a sample containing the analyte with a system as described herein. In some versions, the system employed in the detection method comprises a tethered polypeptide, such as an oxidoreductase, and the detecting includes the oxidoreductase oxidizing the analyte. In some versions, the system employed in the detection method comprises a tethered redox mediator, and the detecting includes the redox mediator undergoing a redox reaction. In some versions, the system employed in the detection method comprises a polymer provided in the form of a hydrogel and an electrode in contact with the hydrogel, and the detecting includes the electrode undergoing a change in electric charge. In some versions, the analyte includes glucose.

Another aspect of the invention includes monomer building blocks useful for making the polymers described above, particularly monomer building blocks suitable for giving rise to the subunits Formula I in the polymers. Such monomers include compounds having a structure of compound 4 or compound 5 or a salt thereof, wherein:
compound 4 and compound 5, respectively, are:

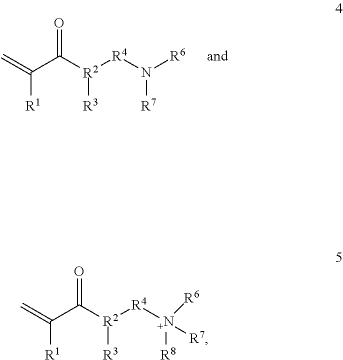

R⁷ is:

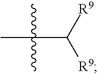

R¹ and R⁸ in each instance are independently H, alkyl, cycloalkyl, aryl, heterocycle, heteroaryl, or a combination thereof;
R² in each instance is independently N or O;
R³ in each instance is independently:
   H; alkyl, cycloalkyl, aryl, heterocycle, heteroaryl, or a combination thereof; or a nitrogen protecting group when R² is N; or
      absent when R² is O;
R⁴ is a spacer arm;
R⁶ is H, alkyl, cycloalkyl, aryl, heterocycle, heteroaryl, or a combination thereof; or R⁷; and
R⁹ is in each instance independently alkyl, alkynyl, cycloalkyl, hydroxyl, halo (e.g., Cl, Br, F), haloalkyl, aryl, aldehyde, halogen-substituted aryl (including aryl groups substituted multiple times with the same or different halogen), nitro-substituted aryl, heterocycle, heteroaryl, hydroxyl, halo, haloalkyl, amino, an amino protected with a nitrogen protecting group, nitro, pyridine, ester, amide, azide, sulfate, sulfite, sulfoalkyl, sulfhydryl, sulfonamide, thiazole, nitro, cyano, alkoxy, carboxy, aldehyde, a saturated or unsaturated cycloalkyl, a saturated or unsaturated heterocycle, bridged saturated and unsaturated cylcoalkyl, fused aromatic, aromatic heterocycle, an N-hydroxysuccinimide ester-reactive group, an amine-reactive group, a sulfhydryl-reactive group, a tethered polypeptide, a tethered redox mediator, a linking arm, a tethered subunit, or a combination thereof.

In various versions of compounds 4 and 5, R¹, R³, R⁶, R⁸, and R⁹ can each independently be substituted or unsubstituted methyl, ethyl, propyl, butyl, propyl, or hexyl. In some versions, at least one of R⁶ and R⁷ in compound 4 is substituted or unsubstituted methyl, ethyl, propyl, butyl, propyl, or hexyl. In some versions at least one of R⁶, R⁷, and R⁸ in compound 5 is substituted or unsubstituted methyl, ethyl, propyl, butyl, propyl, or hexyl.

In some versions of compounds 4 and 5, the spacer arm of R⁴ comprises one or more instances of a moiety selected from the group consisting of —(CH₂)ₘ—, —(CH₂)ₘ—O—(CH₂)ₘ—, —(CH₂)ₘ—(NR¹⁸R¹⁹)—(CH₂)ₘ—, and combinations thereof. R¹⁸ and R¹⁹ in each instance can independently be H; alkyl, cycloalkyl, aryl, heterocycle, heteroaryl, or a combination thereof; or a nitrogen protecting group, with the proviso that at least one of R¹⁸ and R¹⁹ may be absent. Each instance of m can independently be 1-20.

Another aspect of the invention includes methods of making compounds, such as the monomer building blocks described above. An exemplary method includes one or more steps selected from the group consisting of:

a.) reacting compound 1 with compound 2 to yield compound 3, wherein:
compound 1, compound 2, and compound 3, respectively, are:

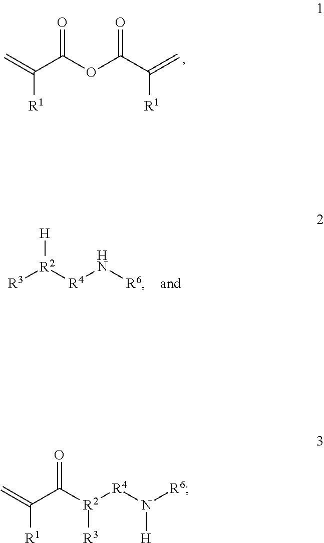

R¹ and R⁶ in each instance are independently H, alkyl, cycloalkyl, aryl, heterocycle, heteroaryl, or a combination thereof;
R² in each instance is independently N or O;
R³ in each instance is independently:
   H; alkyl, cycloalkyl, aryl, heterocycle, heteroaryl, or a combination thereof; or a nitrogen protecting group when R² is N; or
      absent when R² is 0; and
R⁴ is a spacer arm;

b.) reacting compound 3 with compound 6 to yield compound 4, wherein:
compound 6 and compound 4, respectively, are:

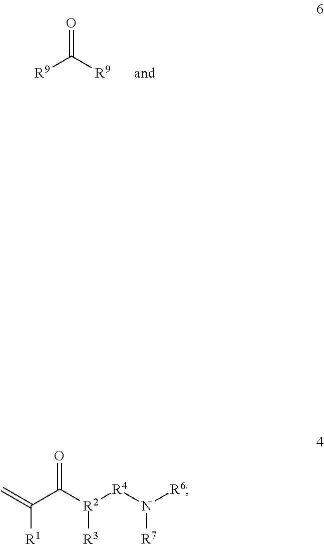

R⁷ is:

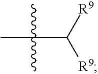

R⁹ is in each instance independently alkyl, alkynyl, cycloalkyl, hydroxyl, halo (e.g., Cl, Br, F), haloalkyl, aryl, aldehyde, halogen-substituted aryl (including aryl groups substituted multiple times with the same or different halogen), nitro-substituted aryl, heterocycle, heteroaryl, hydroxyl, halo, haloalkyl, amino, an amino protected with a nitrogen protecting group, nitro, pyridine, ester, amide, azide, sulfate, sulfite, sulfoalkyl, sulfhydryl, sulfonamide, thiazole, nitro, cyano, alkoxy, carboxy, aldehyde, a saturated or unsaturated cycloalkyl, a saturated or unsaturated heterocycle, bridged saturated and unsaturated cylcoalkyl, fused aromatic, aromatic heterocycle, an N-hydroxysuccinimide ester-reactive group, an amine-reactive group, a sulfhydryl-reactive group, a tethered polypeptide, a tethered redox mediator, a linking arm, a tethered subunit, or a combination thereof; and
R¹, R², R³, R⁴, R⁶, and n in compound 4 are as defined above for compounds 1, 2, and 3, with the proviso that R⁶ in compound 4 can be R⁷ when R⁶ in compounds 2 and 3 is H; and

c.) reacting compound 4 with compound 7 to yield compound 5, wherein:
compound 7 and compound 5, respectively, are:

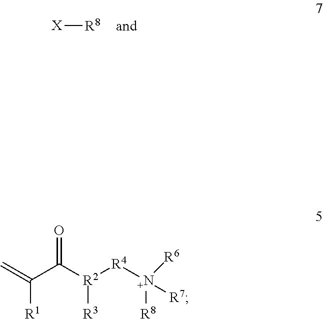

R⁸ is H, alkyl, cycloalkyl, aryl, heterocycle, heteroaryl, or a combination thereof;
X is a leaving group; and
R¹, R², R³, R⁴, R⁶, R⁷ and n in compound 5 are as defined above for compound 4.
   Steps a)-c) are preferably performed under inert atmosphere. Step a) can be performed in an organic, nonpolar solvent such as chloroform (CHCl₃) or others. Other suitable solvents include benzene, toluene, 1,4-dioxane, and dichloromethane, among others. Step b) can be performed in two sub-steps. A first sub-step can involve reaction with a hydride in a polar protic solvent. Suitable hydrides include sodium cyanoborohydride (NaBH₃CN) and sodium triacetoxyborohydride (NaBH(OCOCH₃)₃), among others. Suitable solvents include n-butanol, isopropanol, nitromethane, methanol, and ethanol, among others. A second step can involve addition of a reagent such as acetic acid. Step c) can be performed in a polar aprotic solvent such as tetrahydrofuran (THF). Other suitable solvents include N-methylpyrrolidone, ethyl acetate, acetone, dimethylformamide, acetonitrile, dimethyl sulfoxide, and propylene carbonate, among others. The leaving group in compound 7 is preferably an anionic leaving group, such as halides (e.g., Cl⁻, Br⁻, and I⁻) and sulfonate esters such as tosylate (TsO⁻). Other leaving groups are acceptable.

An ion exchange step can be performed after step c) to provide the monomer with a desired counterion for downstream applications. A preferred counterion for downstream applications is the chloride (Cl⁻) ion. An exemplary method for ion exchange is provided below in the examples.

The scope of additional functional groups for R⁹ in R⁷, compound 6, and Formula IV, beyond those explicitly described herein, can be ascertained from Cheung et al. (Cheung C W, Hu X. Nat Commun. 2016 Aug. 12; 7:12494), Schrittwieser et al. (Schrittwieser J H, Velikogne S, Kroutila W. Adv. Synth. Catal. 2015, 357, 1655-1685), Maya et al. (Maya, R J, Poulose S, John J, Varma, R L. Adv. Synth. Catal. 2017, 359, 1177-1184), Moormann (Moormann A. Synthetic Communications. 1993, 23(6), 789-795), and Ramachandran et al. (Ramachandran P V, Gagare P D, Sakavuyi K, Clark P. Tetrahedron Letters. 2010, 51, 3167-3169).

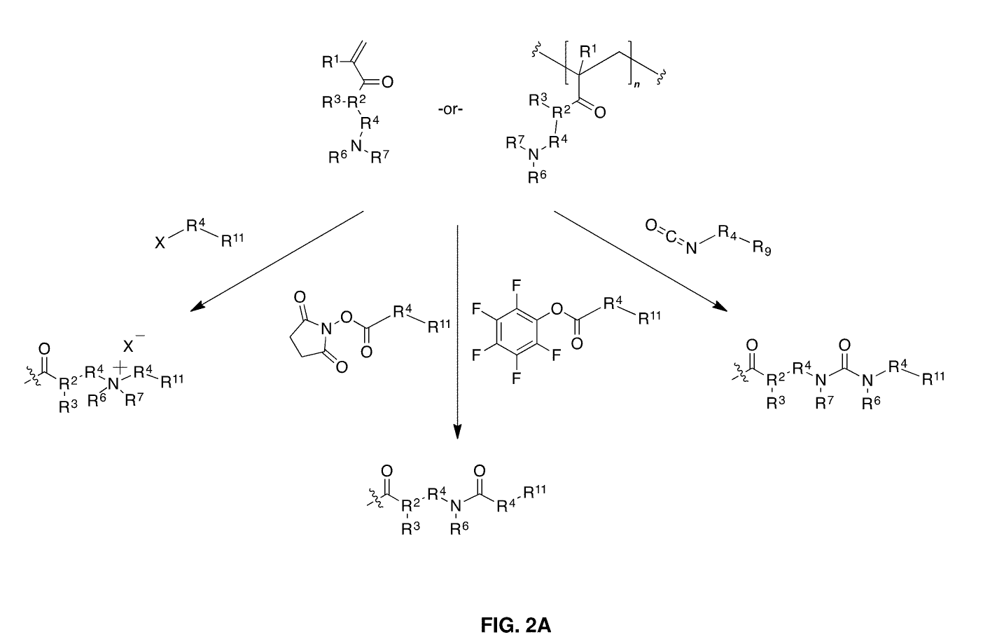

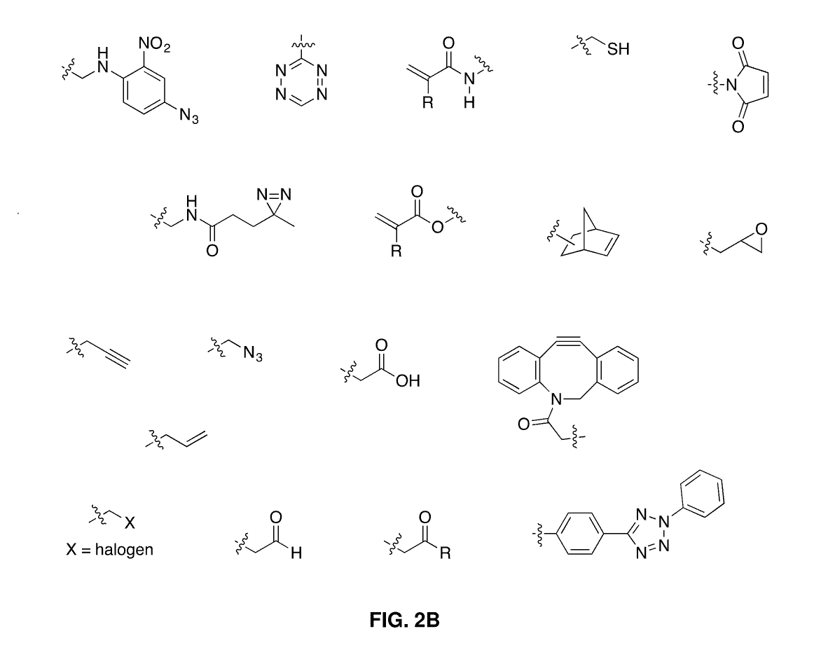

Additional monomers of the invention include monomers that are pre-functionalized to contain a tethering arm, such as those provided in FIGS. 2A and 2B.

Another aspect of the invention is directed to the redox mediators, including the electron shuttles provided by Formulas VI, VII, and VIII, whether tethered to a polymer, tethered to a monomer, tethered to any other component provided herein, or provided in isolation.

Another aspect of the invention is directed to methods of making the systems provided herein. The methods comprise polymerizing monomers to generate a polymer. The monomers can include any one or more monomers provided herein. The polymers can include any one or more polymers provided herein.

In some versions, the monomers comprise terminal amines such as those shown in FIGS. 1A-1C and 3A-3G. Such monomers can form subunits of a polymer after polymerization and can be tethered to other linear polymer chains, enzymes, and/or redox mediators. The subunits can be tethered with the use of tethering agents having two unreacted terminal linking groups. The subunits can also or alternatively be tethered with the use of linear polymer chains, enzymes, and/or redox mediators pre-functionalized with tethering arms comprising a unreacted terminal linking group. Tethering each component can occur simultaneously or sequentially. The tethering can occur with the same or different linking groups.

In some versions, the monomers are pre-functionalized with tethering arms prior to polymerization, such as those shown in FIGS. 2A and 2B. If pre-functionalized with tethering arms prior to polymerization, the monomers can include monomers having the same terminal linking groups or different terminal linking groups. The monomers with different terminal linking groups can be polymerized in various specific proportions with respect to each other. Once polymerized, the monomers form polymer subunits having different terminal linking groups can provide specificity for tethering specific proportions of other components (e.g., enzymes, redox mediators, etc.) depending on the corresponding linking group present on each of the other components. Tethering components can occur simultaneously or sequentially.

Tethering any first component of the invention (e.g., enzyme, redox mediator, tethering agent, tethering arm, first monomer, first polymer etc.) to a any second component of the invention (e.g., enzyme, redox mediator, tethering agent, tethering arm, second monomer, second polymer, etc.) can occur in a number of formats. Some versions include tethering a first component with a tethering arm to a second component lacking a tethering arm by linking the tethering arm of the first component to the second component. Some versions include tethering a first component lacking a tethering arm to a second component with a tethering arm by linking the tethering arm of the second component to the first component. Some versions include tethering a first component with a tethering arm to a second component with a tethering arm by linking the terminal linking groups of the tethering arms to each other (provided the terminal linking groups on the tethering arms are corresponding linking groups). Some versions include tethering a first component lacking a tethering arm to a second component lacking a tethering arm by reacting each with a tethering agent having two unreacted terminal linking groups. Some versions include tethering a first component with a tethering arm to a second component with a tethering arm by reacting each with a tethering agent having two unreacted terminal linking groups that correspond to the unreacted terminal linking groups on the tethering arms.

Other aspects of the invention include any component described herein (monomers, polymers, cross-linked polymers, redox mediators, electron shuttles, enzymes, tethers, tethering agents, tethering arms, etc.), whether provided in isolation or in combination with the other components.

Each variable not explicitly defined in any particular structure or drawing herein (e.g., R¹-R¹⁹, x, n, s, p, etc.) can be defined as in any other particular structure or drawing in which the variable is defined unless the context dictates otherwise. Each instance of the same variable appearing more than once in any given structure is independent of the other instances (e.g., can be different moieties defined for the variable) unless the context dictates otherwise.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### Example 1. Acrylamide and Methacrylamide Monomers Equipped with Pendant PEG Chains Bearing Terminal Nitrogen-Containing Functional Groups

This example describes features, structures, and synthetic preparation of acrylamide and methacrylamide (and related analogs) monomer building blocks. Some of the monomers are equipped with pendant oligo(ethylene glycol) spacer arms bearing terminal nitrogen-containing functional groups (FIGS. 3A-3C). Others have amines and alkylene spacer arms (FIGS. 3D and 3E). The water-soluble monomers are designed and synthesized as building blocks of hydrophilic polymer-based functional materials, namely hydrogels for next generation enzymatic biosensing technologies. The monomers can be prepared in very few synthetic steps using mild conditions and can be readily equipped with a wide range of functional groups.

In general, versatile and functional polymeric materials are not accessible without monomer building blocks equipped with functional groups that facilitate orthogonal chemistries and/or afford unique and modular polymer characteristics.

Functionalized acrylamide and methacrylamide monomers disclosed herein, and their corresponding synthetic routes, provide multi-functional hydrogel materials for enzymatic biosensing. Features of these materials include networks of hydrophilic polymer backbones as well as the ability to immobilize enzymes; to tune mechanical properties, water content, and mass transport properties within the hydrogel materials; and to install active components bound within the polymer network. To exhibit these features, the monomers are composed of acrylamide, alkylacrylamide (e.g., methacrylamide), acrylate, alkylacrylate (e.g., methacrylate), and/or related moieties that yield hydrophilic polymer backbones with, e.g., poly(ethylene glycol) (PEG) or other spacer arm-containing pendants bearing terminal amine or ammonium functional groups. The terminal amine or ammonium functional groups can subsequently act as sites for orthogonal chemistries or other additional functionalities.

FIGS. 1A-1D and 3A-3G illustrate molecular structures of some exemplary monomers of the invention. These include primary or secondary amine-functionalized monomers (FIGS. 1A, 3A, 3D, and 3E) that serve as both functional components for preparation of hydrogel materials and precursors for tertiary amine monomers (FIGS. 1B and 3B) and cationic quaternary ammonium monomers (FIGS. 1C and 3C).

The monomers as shown in FIGS. 1A-1C and 3A-3C can be generated with via a three-step synthetic route as illustrated below in Scheme 1. Each step results in a monomer that serves as the precursor for the next. The synthetic route accesses all three types of monomers in three steps, affords access to an extremely wide range of analogs with an equally broad scope of functional groups positioned at the pendant group terminals, requires no protecting group chemistry, employs mild conditions, requires minimal purification, and provides building blocks of next generation hydrogel targets. An additional fourth step can be employed to interchange the counterions of quaternary ammonium bearing monomers, e.g. a compound 5 monomer (scheme 1 illustrated below) for which iodide is the counterion can be converted to a compound 5 monomer for which chloride is the counterion by way of chloride form ion exchange resins.

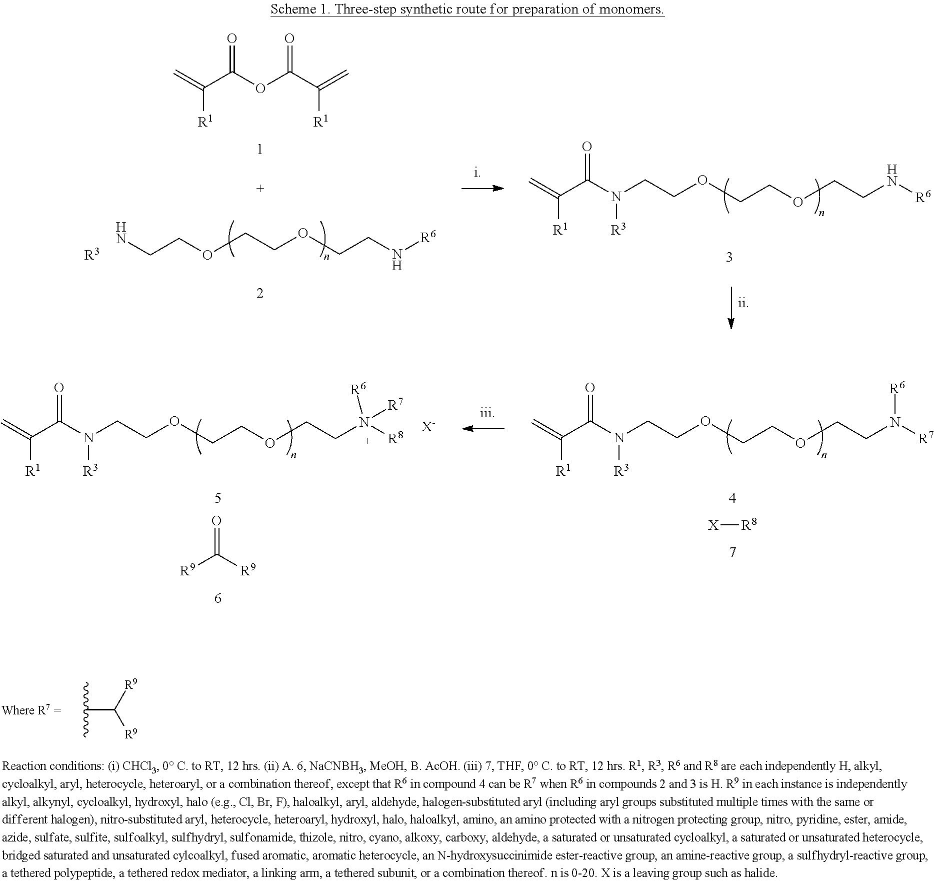

The initial synthetic step is the reaction of acrylic and/or methacrylic anhydrides with an excess of diamine (Scheme 1, compounds 1 and 2, respectively) to yield the primary amine-functionalized monomer (Scheme 1, compound 3). Compound 3 is then converted to tertiary amine compound 4 via a Borch type reductive amination that proceeds by a mechanism that efficiently alkylates the amine group but cannot proceed further to form any quaternary ammonium. In addition to affording compound 4, utilizing the mild reductive amination conditions yields an optimal pathway to cationic compound 5 that avoids the harsh reaction conditions and problematic purifications typically associated with direct conversion of primary amines to quaternary ammoniums. It is important to note that choice of carbonyl compound employed in this reductive amination step allows installation of a myriad of additional nitrogen bound functional groups (note: when compound 6 in Scheme 1 is formaldehyde, the simplest carbonyl compound, group R₃ will be a methyl group). In the final synthetic step, compound 4 is treated with alkyl halide 7 to produce compound 5 in good yield without the requirement of purification. Exemplary synthetic steps of exemplary monomers are described below.

Preparation of an exemplary monomer of compound 3: In a typical procedure, a 250 mL Schlenk flask was equipped with a stir bar, sealed via septum, interfaced with Schlenk manifold, placed under inert atmosphere, and charged with 2,2′-(ethylenedioxy)bis(ethylamine) (5.5 eq., 0.2055 mol, 30.45 g, 30 mL) and CHCl₃ (100 mL). The resulting solution was cooled to 0° C. via ice bath before methacrylic anhydride (1 eq., 37.36 mmol, 5.76 g, 5.56 mL) was added slowly using a syringe with the solution in the reaction flask under vigorous stirring. The resulting reaction solution was allowed to slowly warm to room temperature (as the ice bath melted) and stirred overnight. Upon completion of the allotted reaction time the solvent was removed via roto-vap to afford the crude as a clear oil. The crude material was purified via silica gel column chromatography using a CH₂Cl₂:MeOH:Et₃N (18:3:0.5 by volume) solvent system and afforded the monomer of compound 3 as a light brown viscous oil in 58% yield. ¹H NMR (500 MHz, CDCl₃): δ 6.71 (br s, 1H), 5.66 (m, 1H), 5.27 (m, 1H), 3.57 (s, 4H), 3.55 (t, 2H), 3.49-3.42 (m′s, 4H), 2.90 (s, 2H), 2.84 (t, 2H), 1.91 (m, 3H).

Preparation of an exemplary monomer of compound 4: In a typical procedure a 100 mL Schlenk flask was equipped with a stir bar, sealed via septum, interfaced with Schlenk manifold, placed under inert atmosphere and charged with 3 (1 eq., 4.45 mmol, 0.963 g) and dry MeOH (20 mL). Formaldehyde (10 eq., 44.51 mmol, 3.314 mL of 37 wt. % solution in water containing MeOH as a stabilizer) was then added to the reaction flask via syringe. With the reaction flask under dynamic N₂ pressure the septum was removed (at this point N₂ was purging through headspace), NaCNBH₃ (5 eq., 22.3 mmol, 1.4 g) was added as a solid, and the septum immediately replaced. Addition of NaCNBH₃ results in an exothermic reaction. The reaction solution was stirred for two hours at room temperature while under dynamic N₂ pressure before the septum was removed (leaving the flask unsealed) and AcOH (5.9 eq., 26.2 mmol, 1.57 g, 1.5 mL) was slowly added to the reaction flask and the resulting solution was stirred for ˜1 hr with the flask unsealed. Addition of AcOH results in slow yet observable evolution of gas that persists for ˜0.5 hrs. The reaction solution was stirred with flask unsealed for ˜0.5 hrs after gas evolution had ceased. The reaction solution was then adjusted to pH 8 via addition of 8 M NaOH (aq.) The solution was then transferred into a 250 mL round bottom flask and the MeOH and most of the water was then removed via roto-vap, brine (200 mL) was added to the flask, the resulting solution was transferred to a separatory funnel, and the flask was rinsed with 20 mL portions of CH₂Cl₂ (5×) and a 10 mL portion of water with all washings added to the separatory funnel. The solution in the separatory funnel was extracted with 150 mL of CH₂Cl₂ (3×), the organic phases were combined and dried over MgSO₄, and solvent was removed to afford the monomer of compound 4 as a light brown viscous oil in 72% yield. ¹H NMR (500 MHz, CDCl₃): δ 6.49 (br s, 1H), 5.66 (m, 1H), 5.28 (m, 1H), 3.60-3.53 (m′s, 8H), 3.46 (m, 2H), 2.52 (t, 2H), 2.26 (s, 6H), 1.92 (m, 3H).

The reaction for the preparation of compound 4 is preferably not completely sealed. This prevents pressure buildup due to gas formation. The initial stage run under inert atmosphere is preferably conducted under dynamic N₂, and the second stage (during which the most gas is formed) is preferably performed with the septum removed, leaving the flask unsealed. The reaction is preferably run in a properly functioning fume hood. Additionally, the roto-vap vacuum pump exhaust is preferably routed into fume hood as well. The reducing agent NaCNBH₃ is toxic and can liberate very toxic gas if it comes in contact with acids. Special care is preferably taken to prevent any contact with the user. Special care is also preferably taken to prevent contact with acids and to prevent storage and handling in the presence of incompatible materials.

Preparation of an exemplary monomer of compound 5: In a typical procedure, a 100 mL round bottom flask was equipped with a stir bar, charged with a monomer of compound 4 (1 eq., 3.22 mmol, 0.786 g), sealed via septa, and placed under inert atmosphere by purging the headspace with N₂. Dry THF (35 mL) was added to the reaction flask via cannula, and the resulting solution was cooled to 0° C. via ice bath. Methyl iodide (1 eq., 3.22 mmol, 0.457 g, 0.2 mL) was added dropwise using a syringe with the solution in the reaction flask under vigorous stirring. The resulting reaction solution was allowed to slowly warm to room temperature (as the ice bath melted) and stirred overnight. The product precipitates as the reaction proceeds. Upon completion of the allotted reaction time the solvent was removed via roto-vap to afford the monomer of compound 5 as an extremely thick, viscous oil in nearly quantitative yield. 1H NMR (500 MHz, D20): δ 5.78 (m, 1H), 5.55 (m, 1H), 4.05 (m, 2H), 3.79 (s, 4H), 3.75 (t, 2H), 3.66 (m, 2H), 3.54 (m, 2H), 3.26 (s, 9H), 2.01 (m, 3H).

Example procedure for exchange of counterions of an exemplary monomer of compound 5: In order to exchange the counterion of an exemplary monomer of compound 5 from iodide counterions to chloride counterions Amberlite IRA-400 chloride form ion exchange resin (˜20-40 g) was first loaded into an Erlenmeyer flask followed by ˜250 mL of Milli-Q water and washed by swirling, allowing the resin beads to settle to the bottom of the flask, and decanting the water from the flask. The freshly washed resin was then loaded into a glass chromatography column, further washed by passing ˜200 mL of Milli-Q water through the column, activated by passing ˜200 mL of 1 M HCl (aq.) through the resin containing column, and rinsed by passing ˜300-400 mL of Milli-Q water though the column. The solvent was then switched to methanol by first draining the water from the column followed by successively passing ˜100-150 mL aliquots of methanol through the column. Compound 5 with iodide counterions (6.067 g) was dissolved in a minimal amount of methanol, added to the ion exchange resin column, and slowly passed over the ion exchange resin by securing the column stopcock to allow a steady drip of solution to elute the column. The methanolic eluant was collected and the solvent removed to afford compound 5 with chloride counterions in 99% yield. 1H NMR (500 MHz, D₂O) chemical shifts were observed to be the same as those corresponding to compound 5 with iodide counterions.

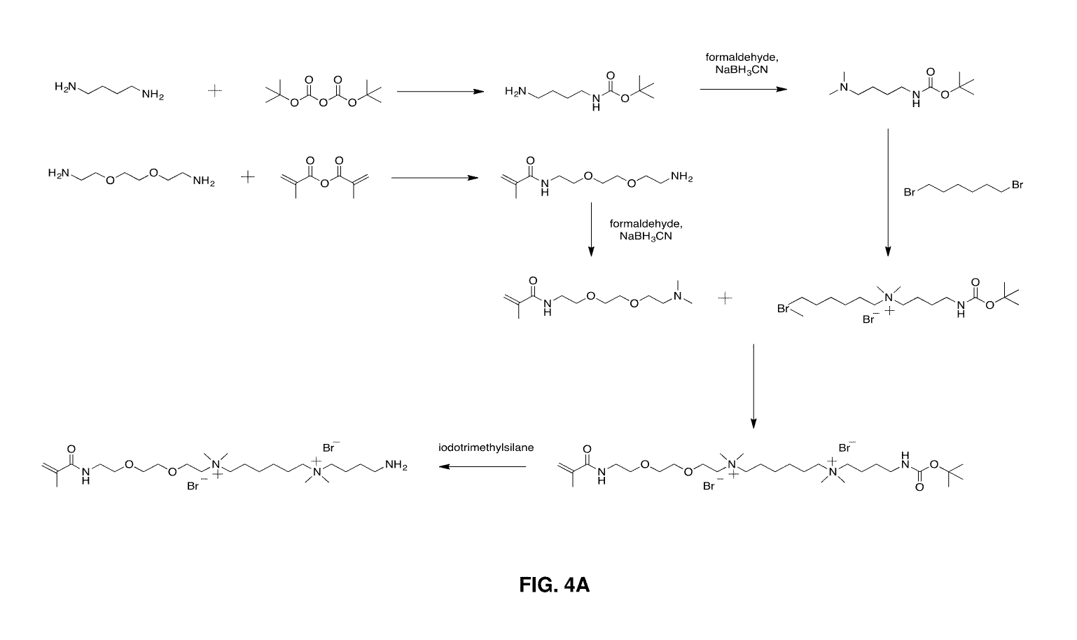

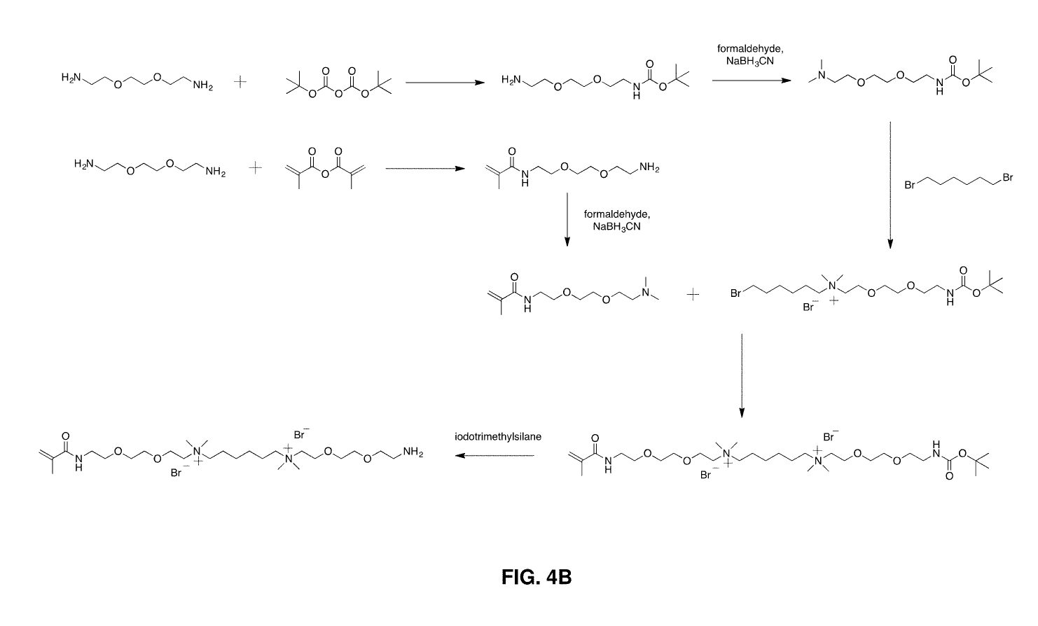

Synthesis schemes for generating the monomers shown in FIGS. 3D and 3E are provided in FIGS. 4A and 4B, respectively.

Additional monomers of the invention include amine-containing monomers that are modified to contain a tethering arm prior to polymerization. See, e.g., FIGS. 2A-2B.

Acrylate-based and alkylacrylate monomers corresponding to the acrylamide and alkylacrylamide moieties described herein are also encompassed by the invention and can be generated according to similar methods.

Acrylamide, alkylacrylamide, acrylate, alkylacrylate, and their functionalized analogs are generally water soluble, are among the very best monomers for hydrogel formation, and can be polymerized via several types of polymerizations including free radical polymerization and reversible addition-fragmentation chain-transfer polymerization (RAFT). When polymerized in the presence of cross-linkers or cross-linked following polymerization, the resulting three-dimensional networks nearly always exhibit hydrogel properties due to the hydrophilicity of their polymer chains. Preparation of substituted and/or functionalized acrylamides and methacrylamides can be achieved in good yields via several routes starting from cost effective and commercially available materials.

The monomers of the invention described herein have many favorable features: 1) acrylamide/methacrylamide moieties bearing PEG pendants with terminal functionality maintain water solubility of the monomers (a property that can be lost or negatively impacted if alkyl chain pendants are employed); 2) hydrophilicity of the corresponding polymer hydrogel materials is not diminished as a result of pendant functionality; and 3) the availability of diaminated PEG “oligomers” of well-defined lengths lends ease and cost effectiveness to the synthetic preparation of monomers of this architecture.

The choice of acrylamide- and methacrylamide-type monomers permits exploiting the utility of nitrogen-containing functional groups (in this case amines) for both forming acrylamide moieties and for creating suitable monomer building blocks for multi-functional hydrogel materials. These hydrogels are equipped with both reactive sites, useful for immobilizing enzymes and tethering redox species, and ionic functional groups that can facilitate more favorable electrostatic interactions between the hydrogel network and the hosted enzymes. Improving the electrostatic environment of the enzyme serves to aid in stabilization and prevent phase separation. Acrylamide/methacrylamide preparation based on the reaction of PEG diamines with acrylic/methacrylic anhydrides (Scheme 1, step i) results in the target monomer framework, a reactive functional group on the pendant terminals, and flexible pendant chains. The flexibility of the chains lends utility to the corresponding hydrogel materials by allowing the pendant functional groups increased range of motion (relative to such groups bound in close proximity to polymer backbones) to help promote favorable reactions and interactions.

### Example 2. Synthesis of Ortho-Naphthoquinone-4-(Piperazine)Sulfonamide Electron Shuttle (NQSA-2)

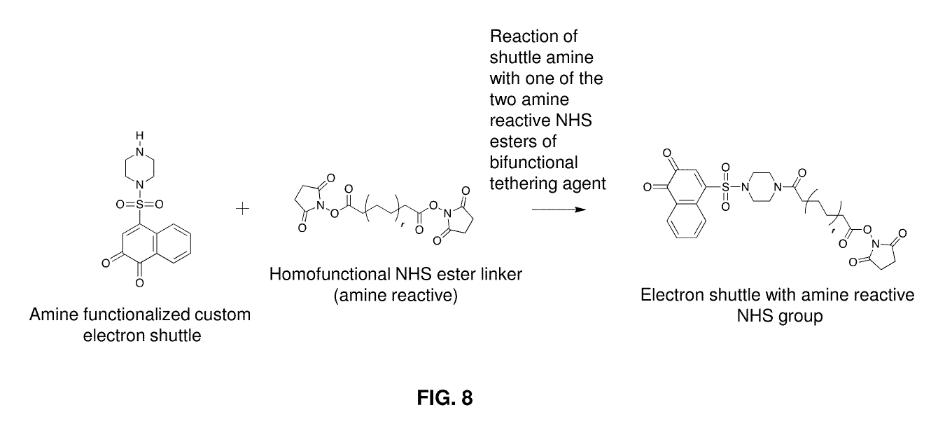

The procedure synthesizing NQSA-2 (see FIG. 8 for structure) was based on J. Am. Chem. Soc. 2004, 126, 1024-1025. A 1000-mL Schlenk flask was charged with 1,2-naphthoquinone-4-sulfonic acid sodium salt (1 eq., 6.36 g, 24.44 mmol) and tetrabutylammonium chloride (1 eq., 6.79 g, 24.44 mmol), equipped with a stir bar, sealed via septum, interfaced with a Schlenk manifold, and placed under inert atmosphere. Dry CH₂Cl₂ (˜500 mL) was added via cannula, and the resulting mixture, initially an orange suspension, was rigorously stirred for 2 hours, at which time the mixture in the flask had become an orange solution of 1,2-naphthoquinone-4-sulfonic acid tetrabutylammonium salt containing a suspension of NaCl in the form of a fine white solid. A separate 250-mL Schlenk flask was charged with triphenylphosphine oxide (2.2 eq., 15.0 g, 53.8 mmol) equipped with a stir bar, sealed via septum, interfaced with a Schlenk manifold, and placed under inert atmosphere. Dry CH₂Cl₂ (˜140 mL) was added via cannula, and the solution was stirred until all solids were dissolved. Trifluoromethanesulfonic anhydride (triflic anhydride; 1 eq., 24.4 mL of 1 M soln in CH₂Cl₂, 24.4 mmol) was added via syringe, and the resulting solution was stirred at room temperature for 15 minutes. The reaction of triphenylphosphine oxide with trifluoromethanesulfonic anhydride to form triphenylphosphine ditriflate was first accompanied by a pink/purple coloration of the reaction solution followed by the formation of a fine white precipitate. The suspension of triphenylphosphine ditriflate was transferred via cannula to the 1000-mL Schlenk flask containing the solution of 1,2-naphthoquinone-4-sulfonic acid tetrabutylammonium salt, and the resulting solution was stirred at room temperature for 30 minutes before being cooled to 0° C. via ice bath. During this 30 minute reaction period, a clean, dry 250-mL Schlenk flask was charged with piperazine (4 eq., 8.42 g, 97.8 mmol), equipped with a stir bar, sealed via septum, interfaced with a Schlenk manifold, and placed under inert atmosphere. CH₂Cl₂ (˜125 mL) was added via cannula. Anhydrous Et₃N (4 eq., 9.9 g, 13.6 mL, 97.8 mmol) was added via degassed syringe, and the solution was stirred until all solids were dissolved. With the solution of activated 1,2-naphthoquinone-4-sulfonate cooled to 0° C., the solution of piperazine and Et₃N was transferred into the 1000-mL reaction flask via cannula as rapidly as possible, and the solution was stirred while slowly warming to room temperature and stirring continued overnight at room temperature. Generally, the proper order of addition would be to add the activated sulfonate to the piperazine solution. However, the order was reversed in this case due to the presence of undissolved solids that rendered transfer of the activated sulfonate solution problematic; we acknowledge that the order of addition surely had a negative impact on the reaction yield. Upon completion of the allotted reaction time, the solution was concentrated by purging the headspace with nitrogen until the volume was reduced to ˜450-500 mL. The solution was transferred to a 1000-mL separatory funnel, ˜250 mL of 2 M HCl (Aq.) and ˜150 mL D.I. water was added, and the contents were rigorously shaken resulting in protonation and partitioning of the target product into the aqueous phase. The organic phase was separated and the aqueous phase was washed 3× with CH₂Cl₂ (˜350 mL/wash). A ˜400-mL volume of CH₂Cl₂ was added to the separatory funnel before addition of a volume of 8 M NaOH (Aq.) sufficient to render the aqueous phase to pH ˜10-12, thereby deprotonating the target product for partitioning back into the organic phase. The aqueous phase was extracted 3× with CH₂Cl₂ (˜350 mL/wash), the combined organic phases were dried with MgSO₄, and solvent was removed by rotary evaporation to afford the crude product as a red solid. Purification via silica gel column chromatography using base treated silica gel and CH₂Cl₂ mobile phases with low percentages of MeOH afforded the pure product as a blood red solid in 25-40% yields. ¹H NMR (500 MHz, CDCl₃): δ 8.11 (d, J=7.55 Hz, 1H), 7.64 (t, J₁₋₂=8.00 Hz, J₁₋₂=7.25 Hz, 1H), 7.58 (d, J=7.65 Hz, 1H), 7.53 (t, Jⱼ₋₂=7.65 Hz, Jⱼ₋₂=7.35 Hz, 1H), 6.01 (s, 1H), 3.37 (t, 4H), 3.11 (t, 4H).

### Example 3. Functional Hydrogel Materials

This example describes hydrogel materials made with monomers such as those described in Example 1 and, optionally, electron shuttles such as that described in Example 2. The hydrogel materials are useful for next generation enzymatic biosensors. The hydrogel materials can include redox hydrogel functional materials containing oxidoreductase and/or non-oxidoreductase enzymes useful for next generation electrochemical enzymatic biosensors. The hydrogel materials can also or alternatively include non-redox hydrogel functional materials that contain oxidoreductase and/or non-oxidoreductase enzymes. The materials are prepared from acrylamide, methacrylamide, acrylate, and/or methacrylate monomers with pendant functional groups (such as those described in Example 1) either alone or in combination with non-functionalized acrylamide, methacrylamide, acrylate, and/or methacrylate co-monomers. The pendant functional groups can include, for example, amine groups, cationic functional groups, and/or other functional groups, such as linking groups attached via amino groups. The resulting materials include hydrophilic polymeric materials decorated with reactive sites and functional groups that can be modified for a wide range of specific applications. Depending on the pendant functional groups and their modifications, the materials can facilitate signal transduction in sensing systems by serving to immobilize enzyme components, regulate mass transport rates of analytes and other dissolved compounds that participate in device function, and, in some versions, mediate transport of electrons from oxidoreductase enzymes to the sensing electrodes.

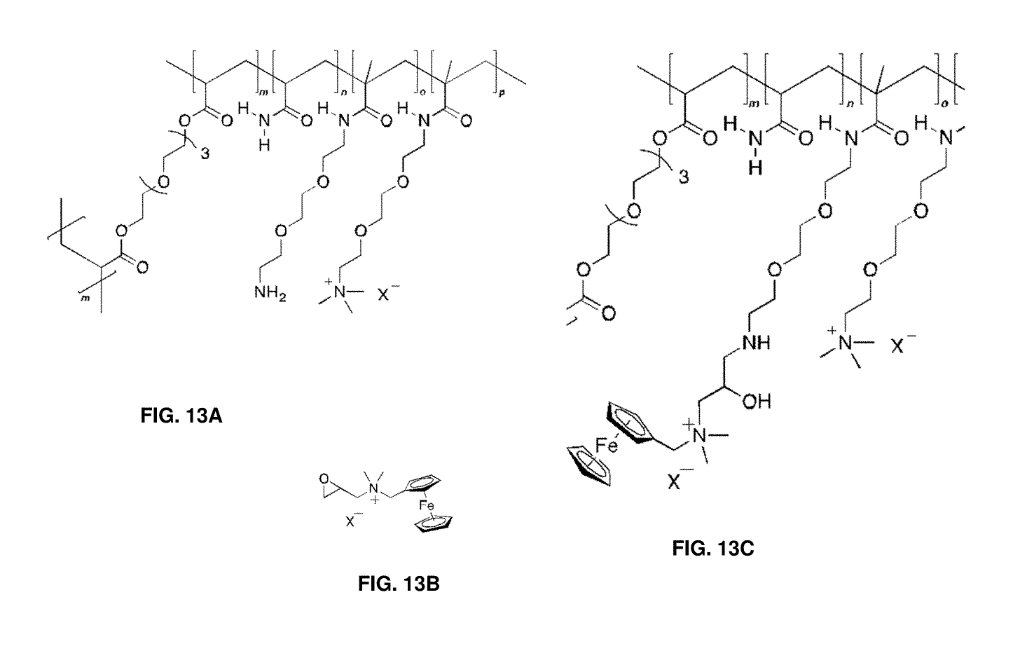

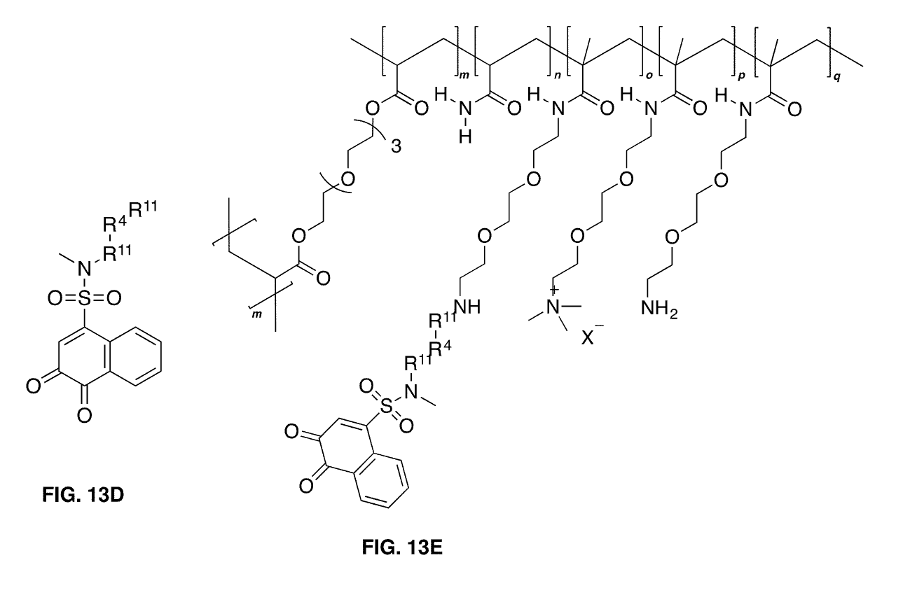

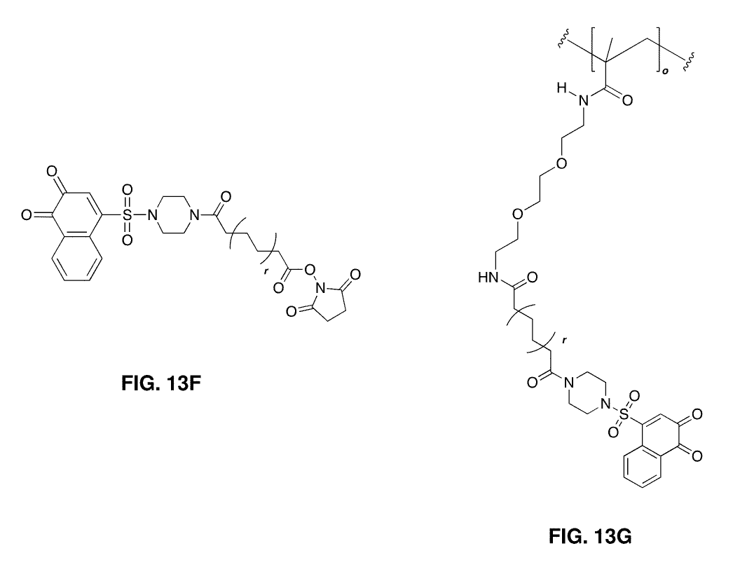

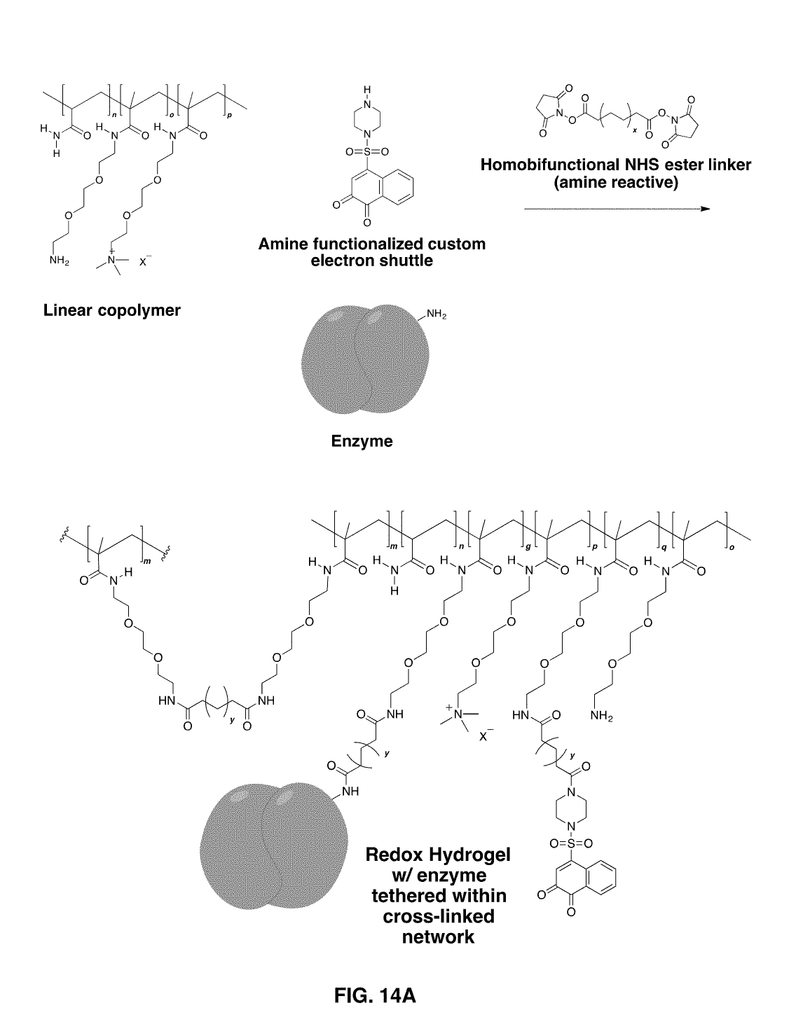

FIGS. 13A, 13C, 13E, and 13G illustrate structures of exemplary hydrogel materials (without tethered enzymes, which can be included). Each combines key features of both solids and liquids (a feature of hydrogels), is readily tunable, affords stabilizing effects for biological enzyme catalyst components, can be readily formed in aqueous conditions compatible with enzyme components (i.e., that preserve enzyme activity), and is amenable to a wide range of processing methods. These multifunctional materials are well suited for next generation sensing technologies such as those designed for detection using very small sample volumes.

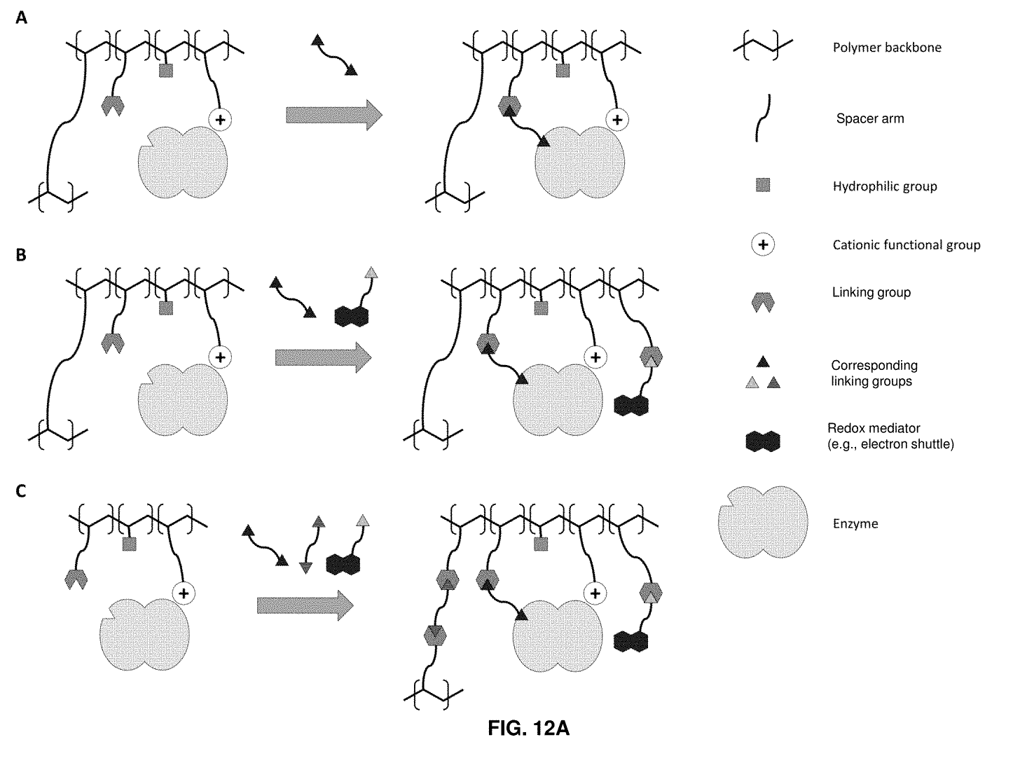

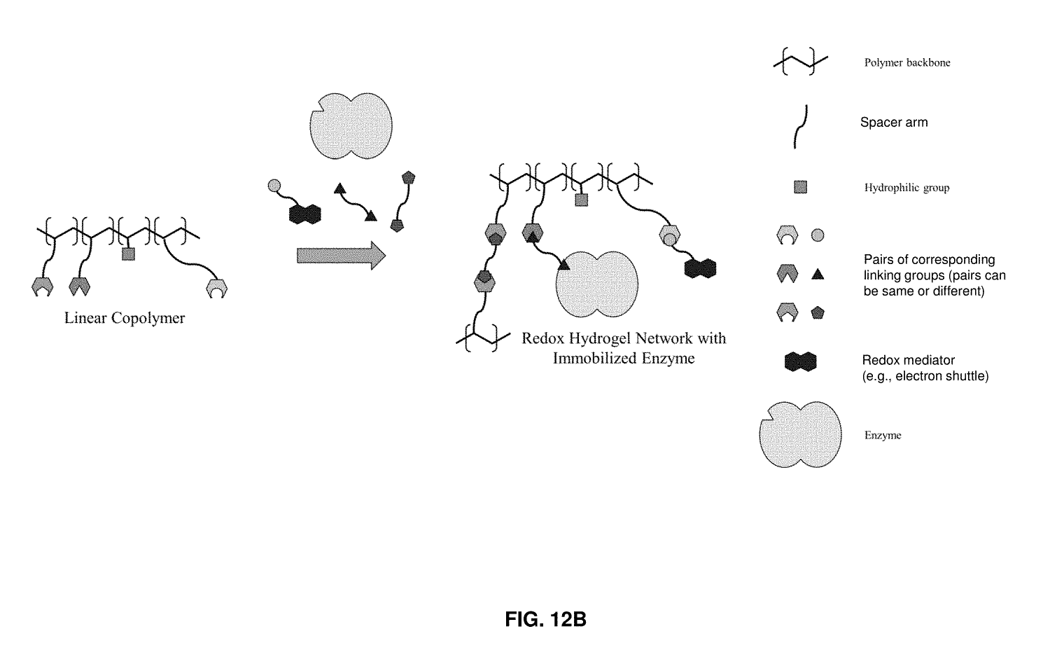

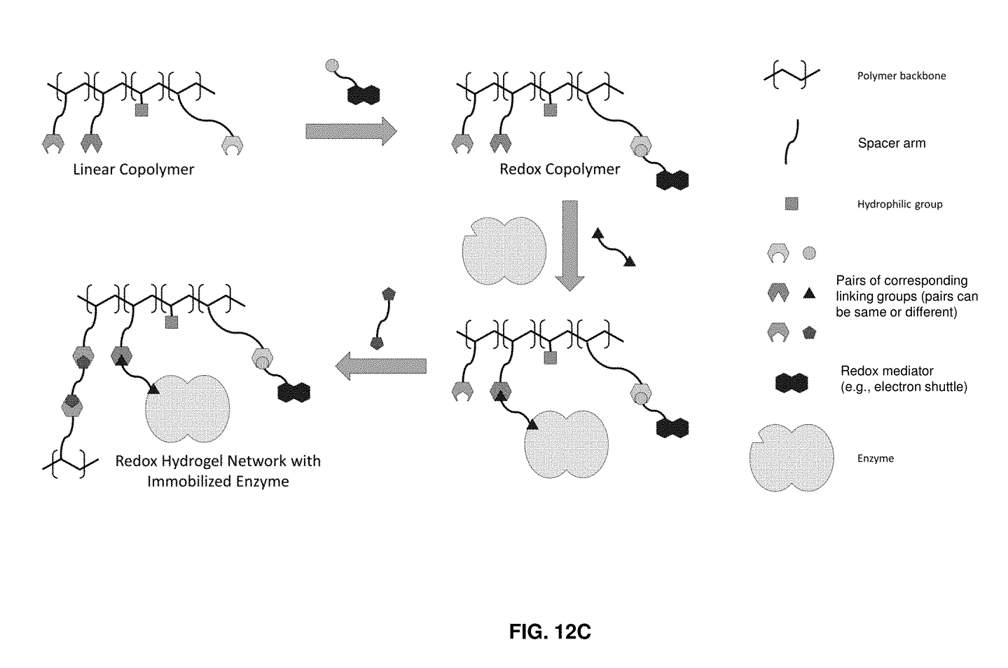

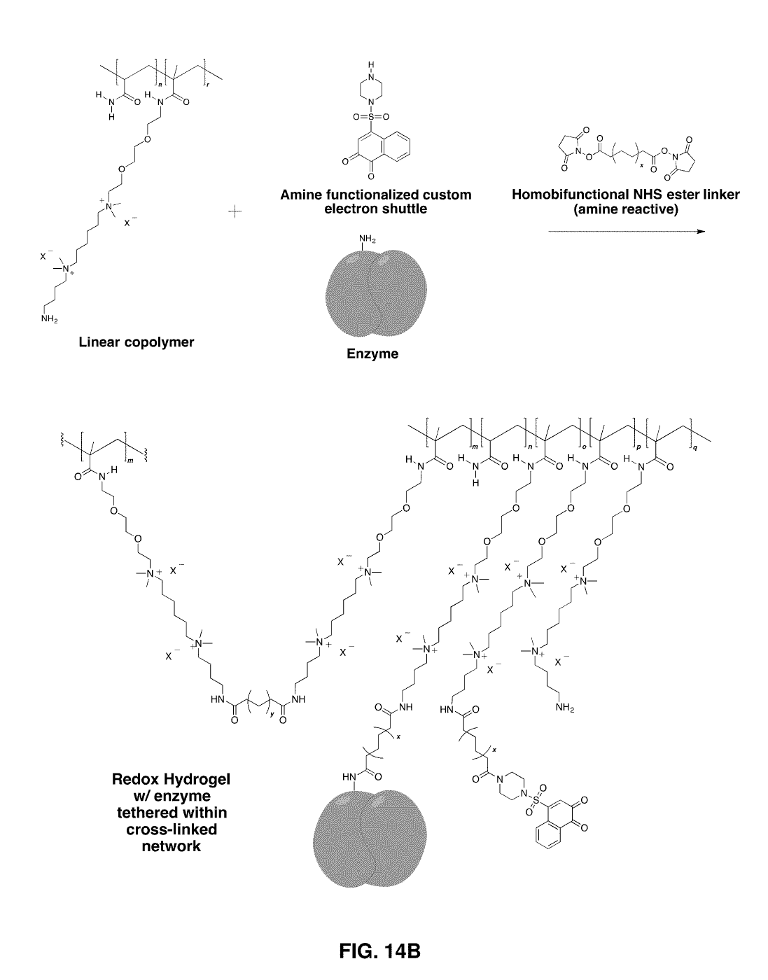

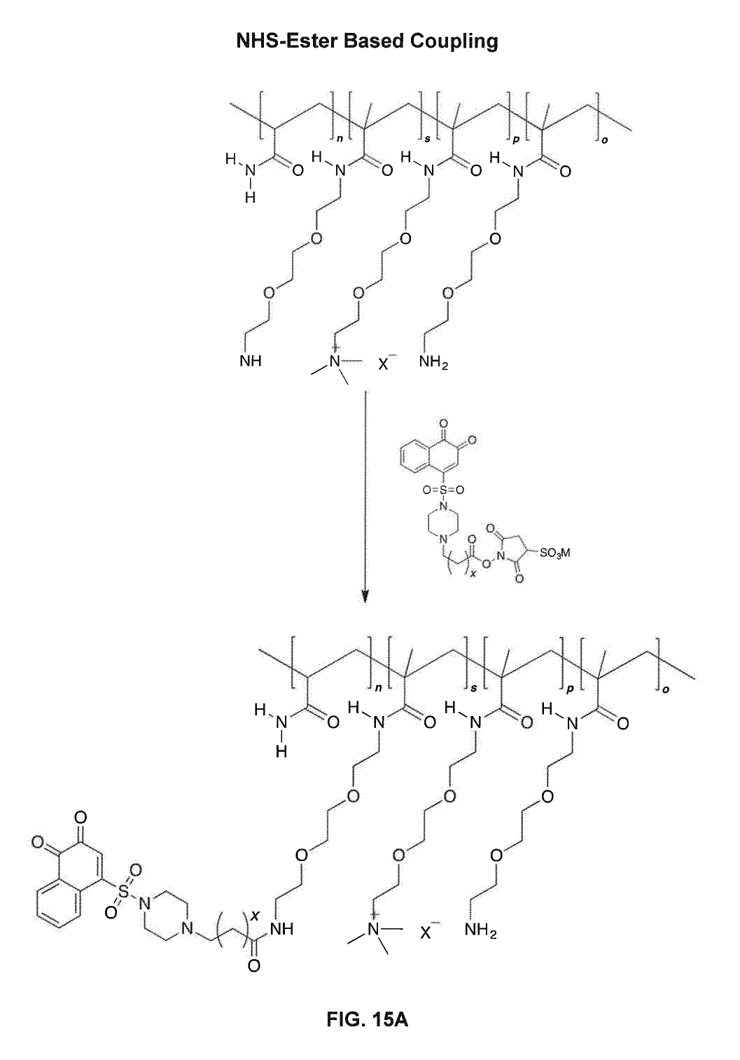

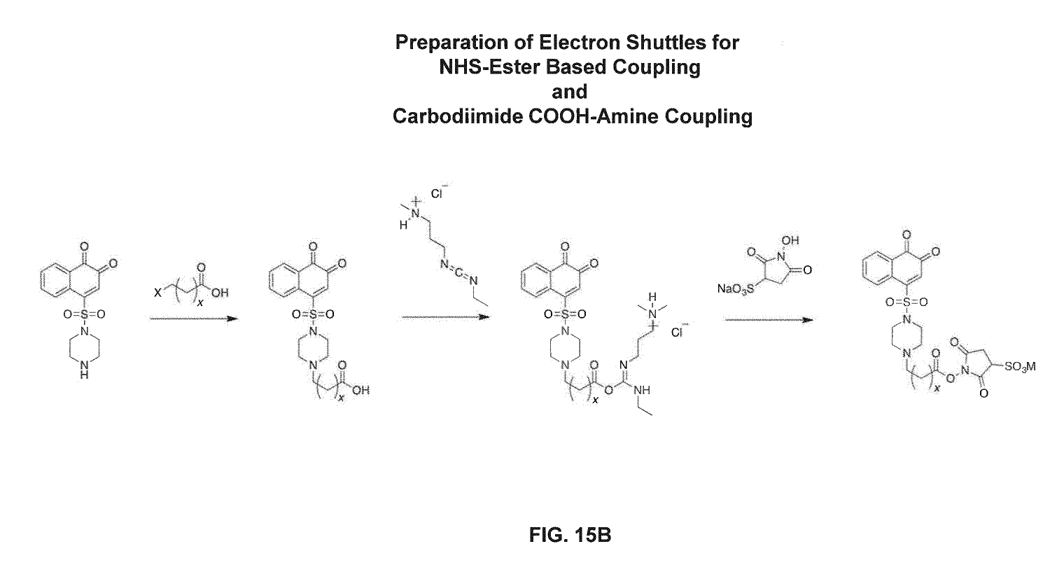

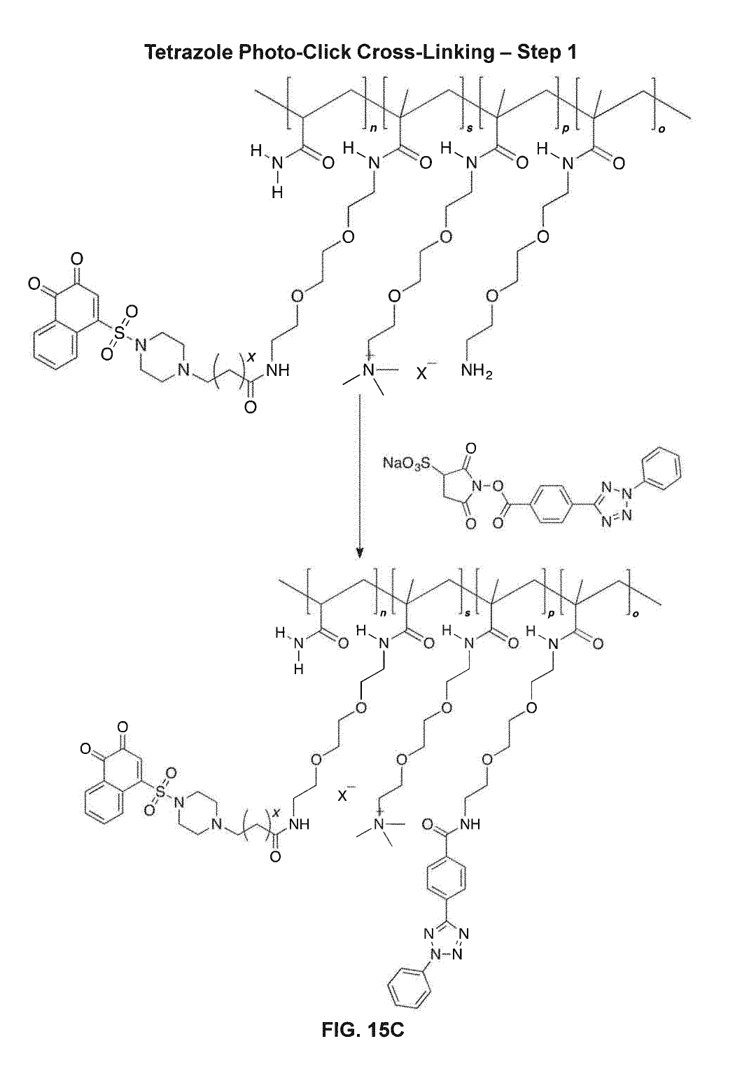

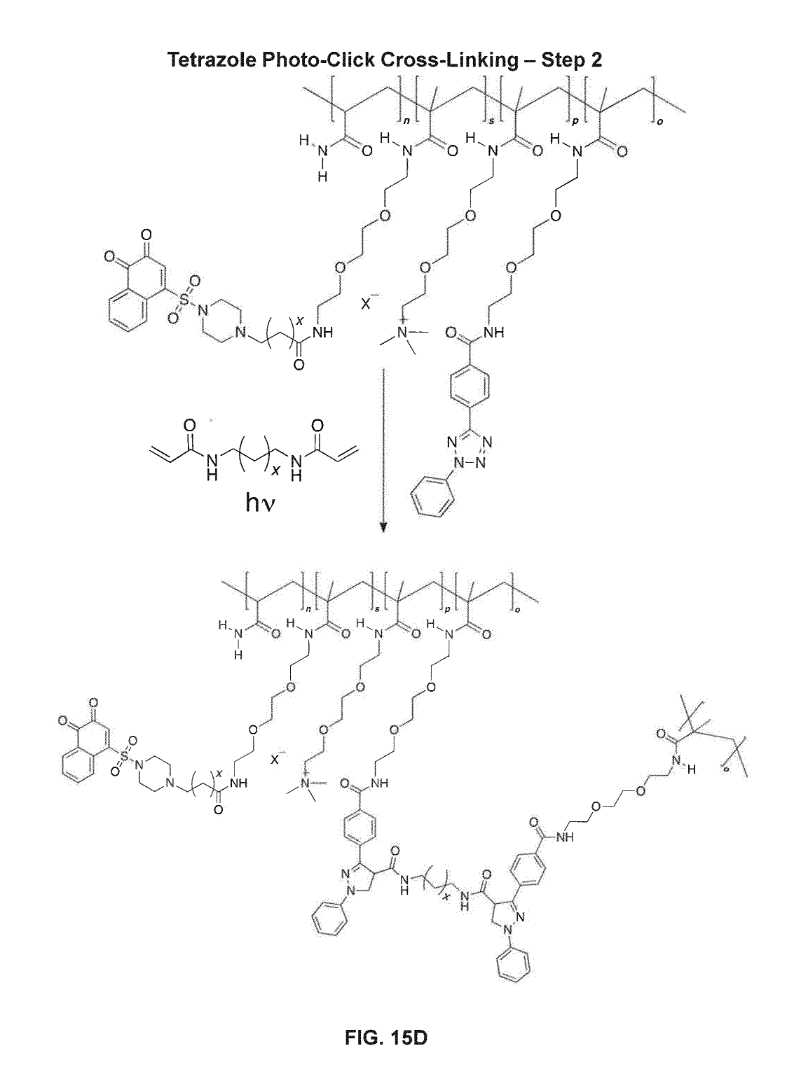

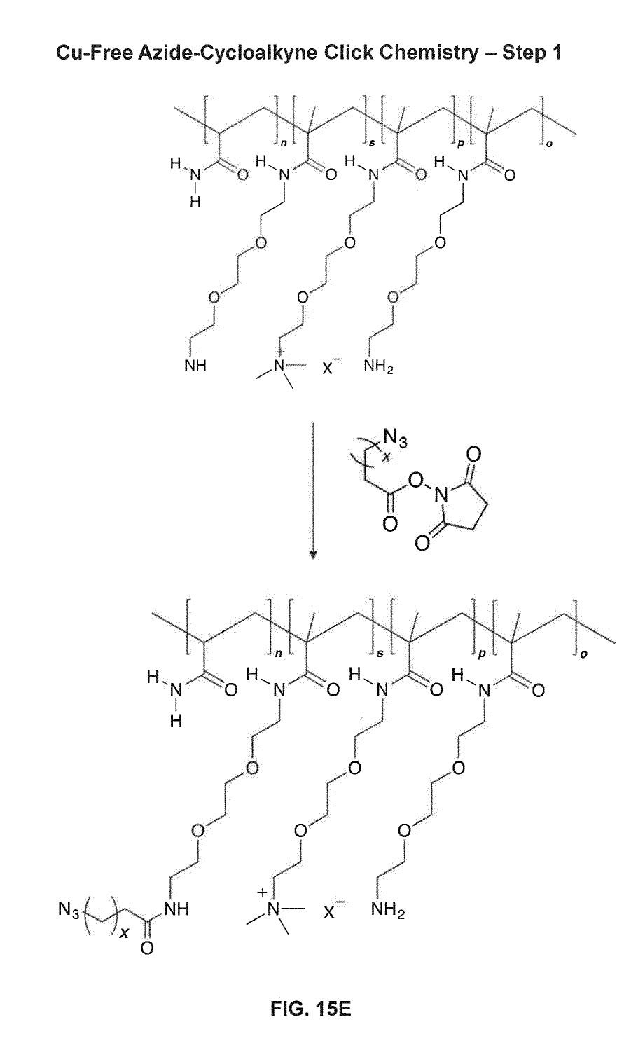

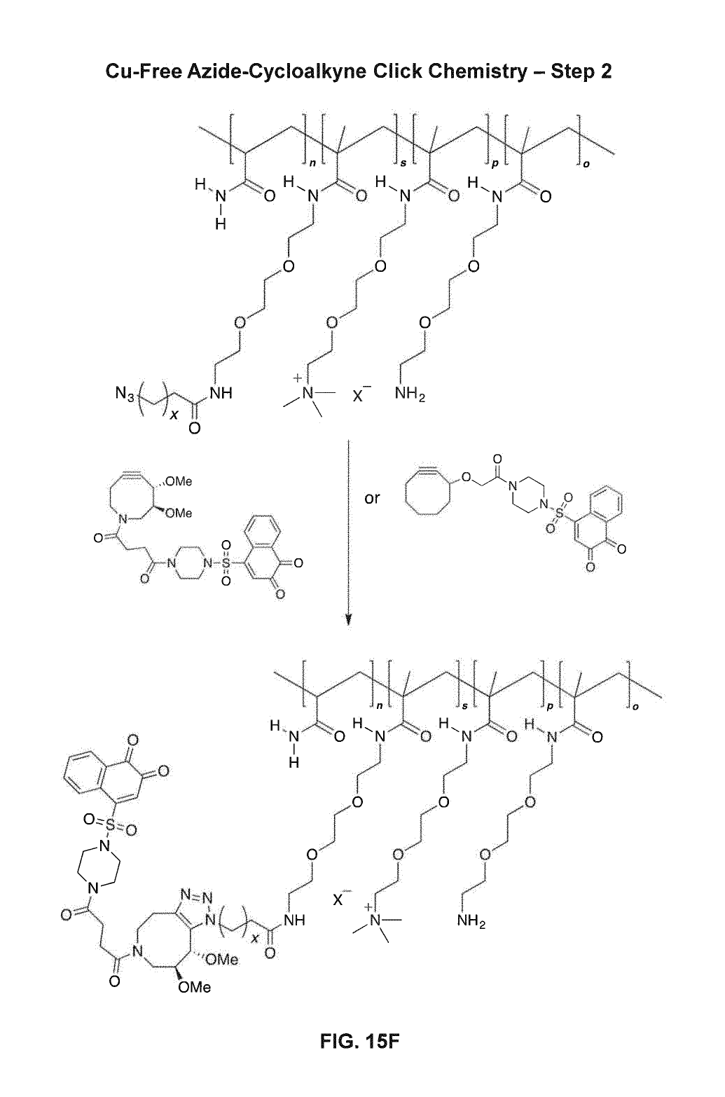

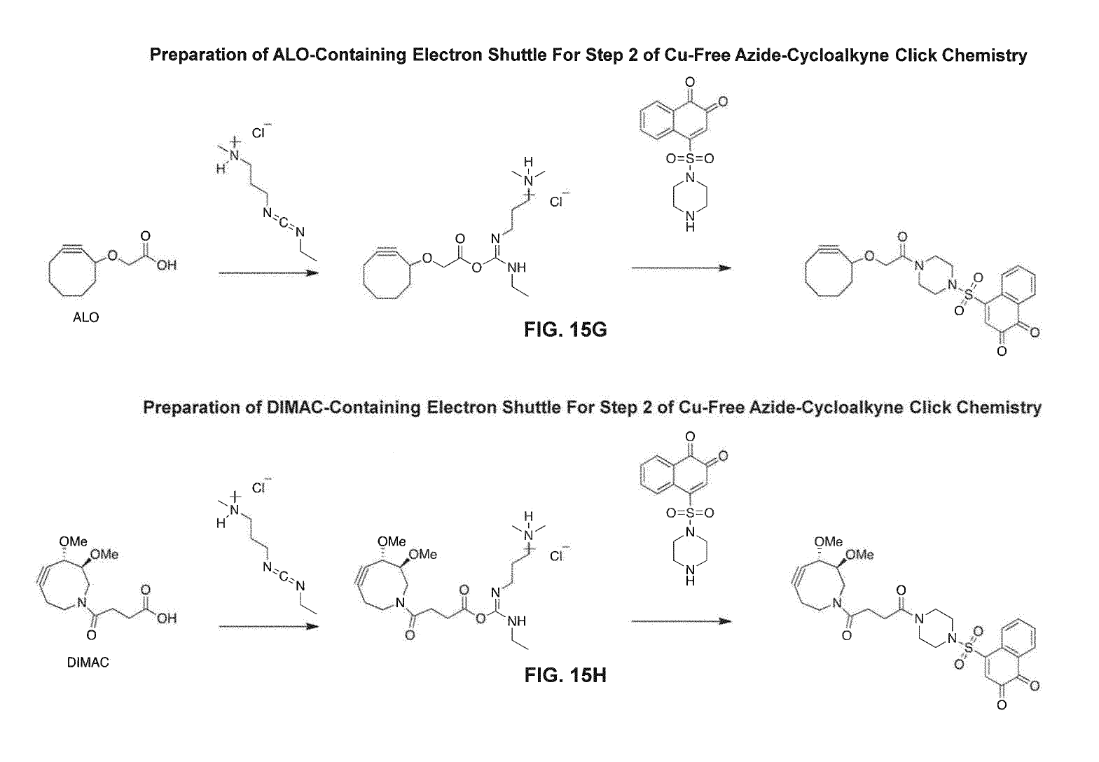

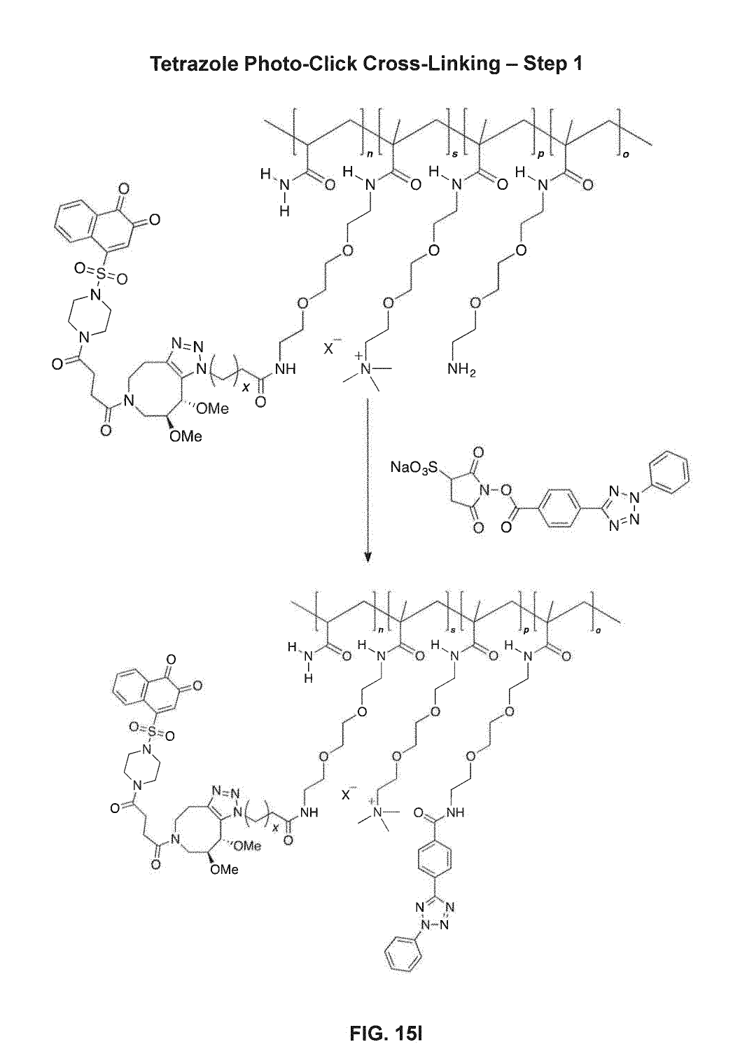

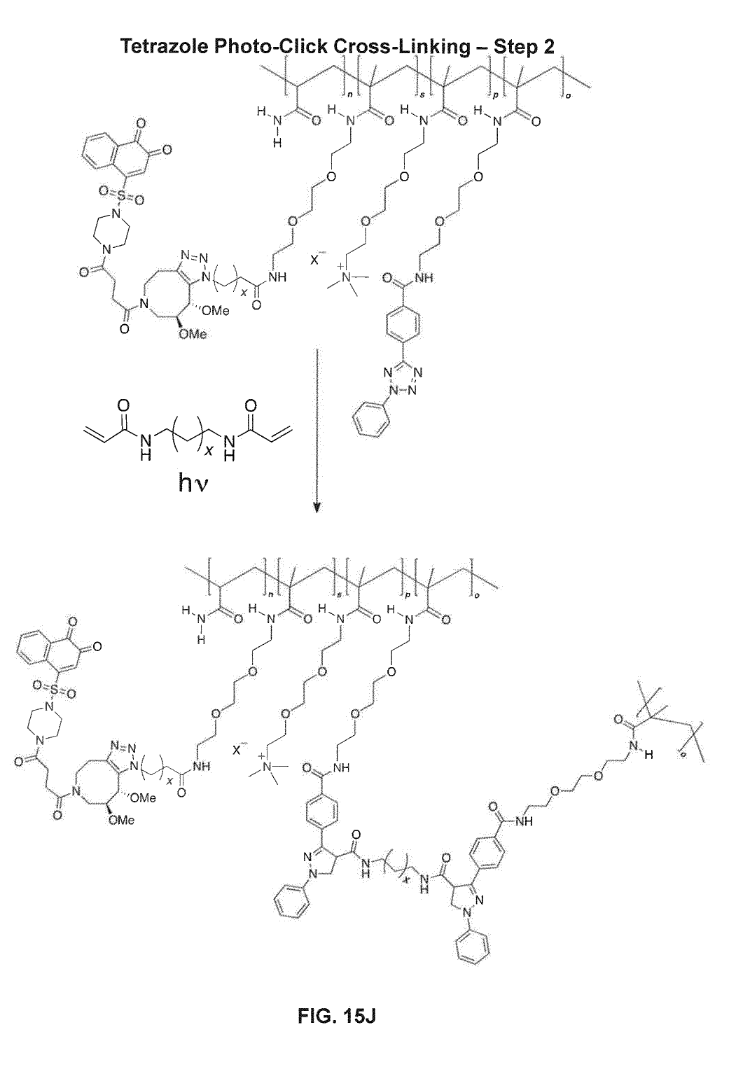

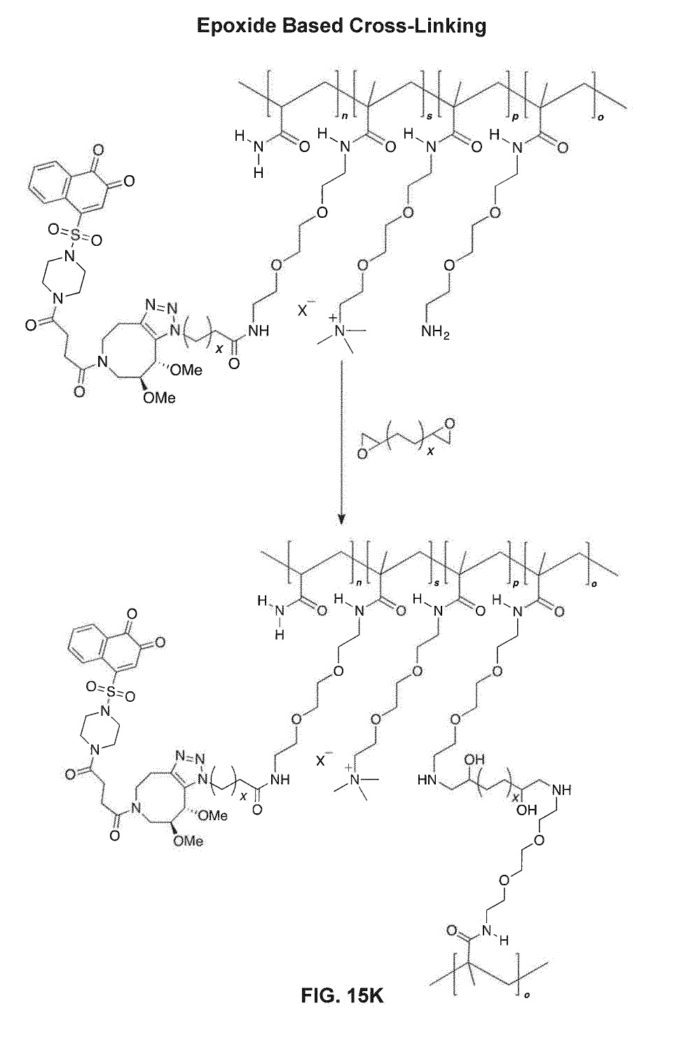

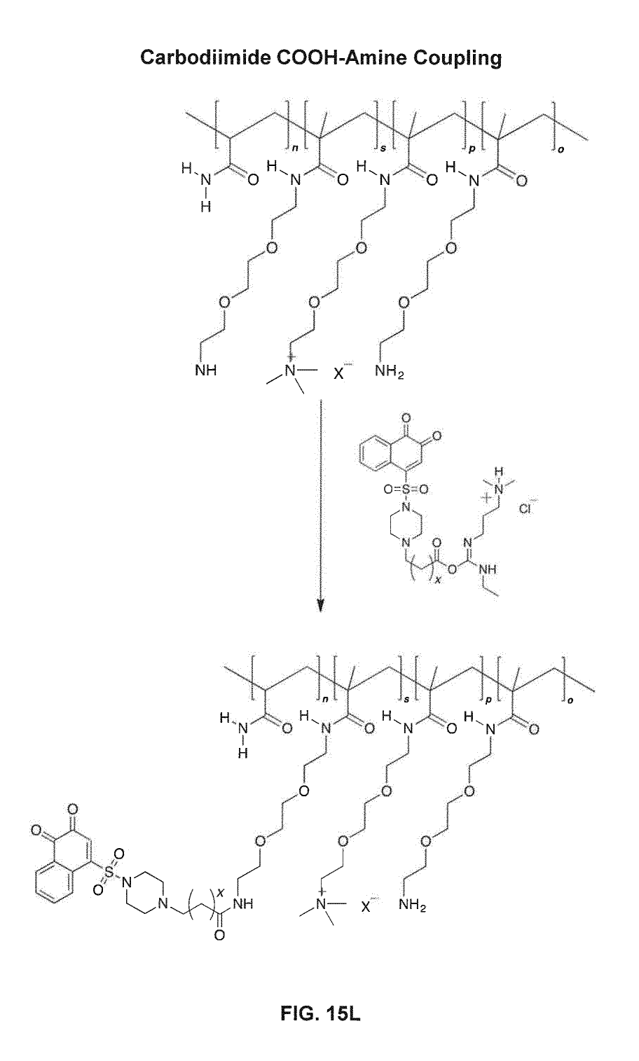

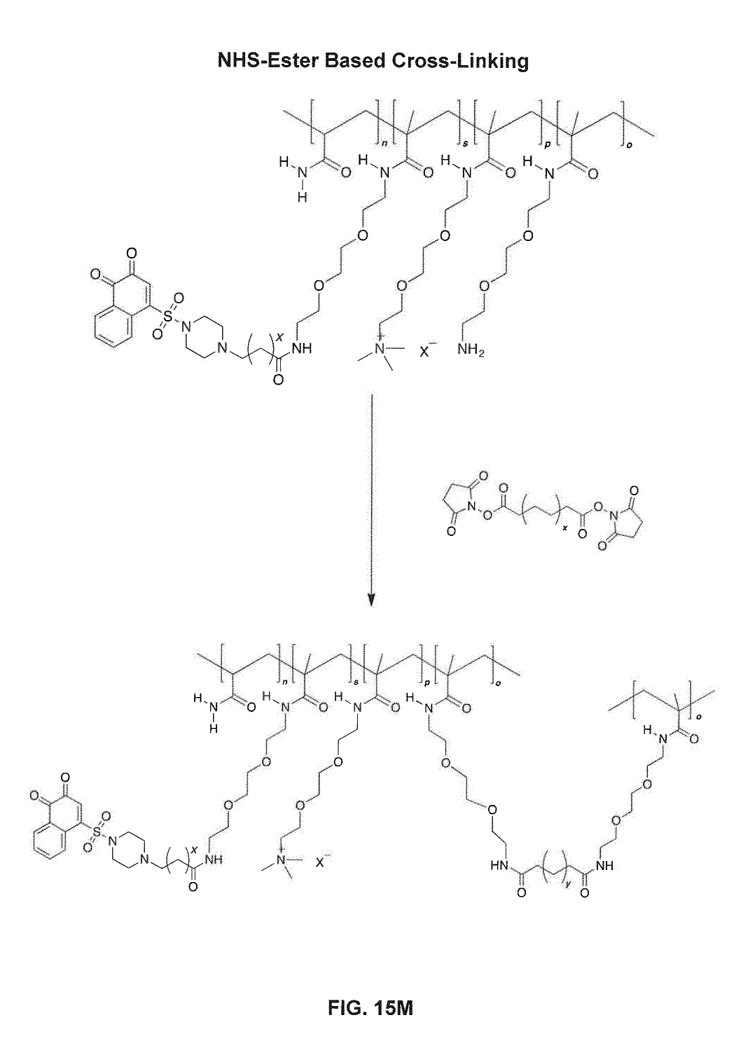

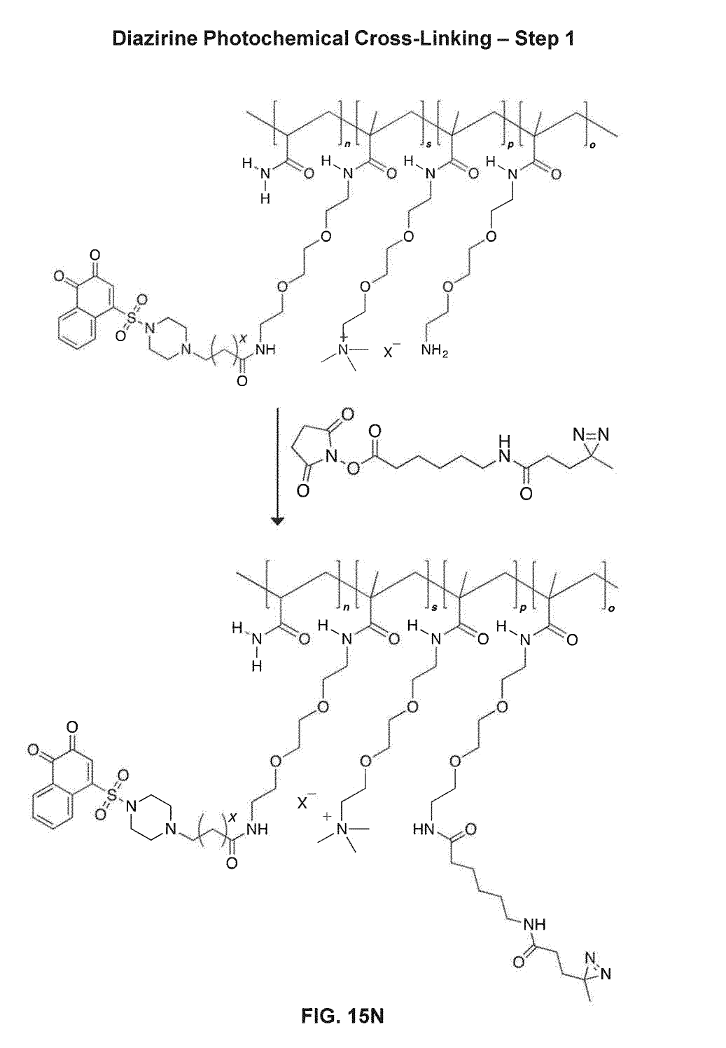

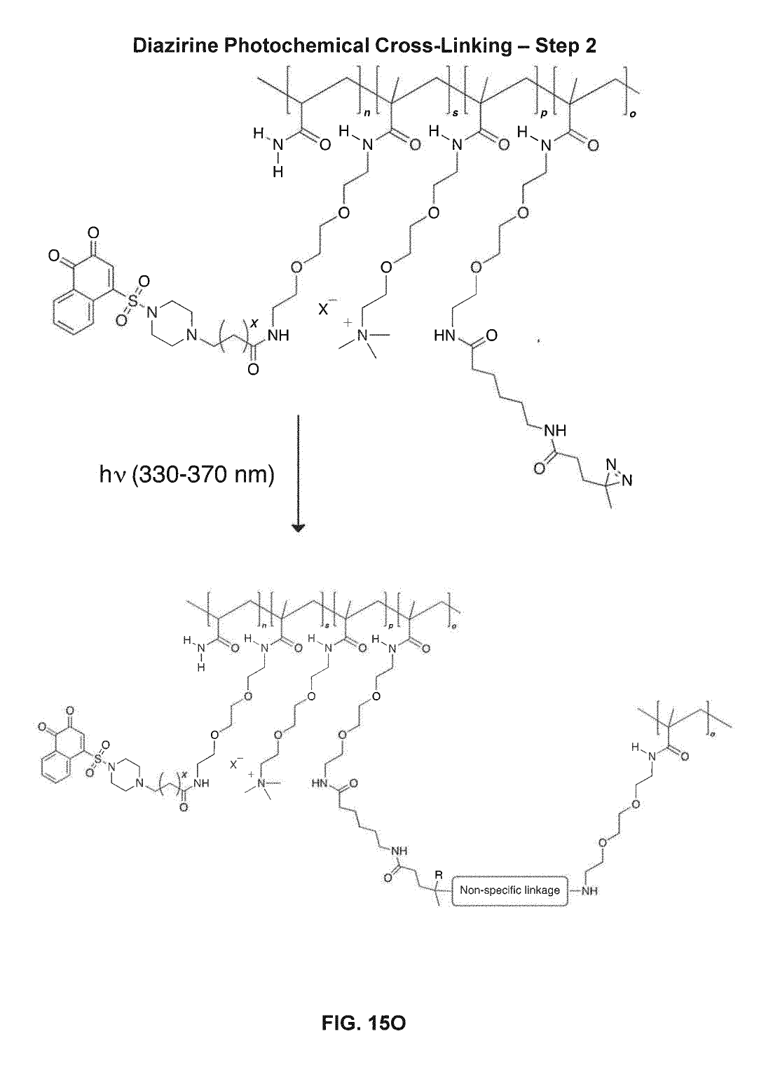

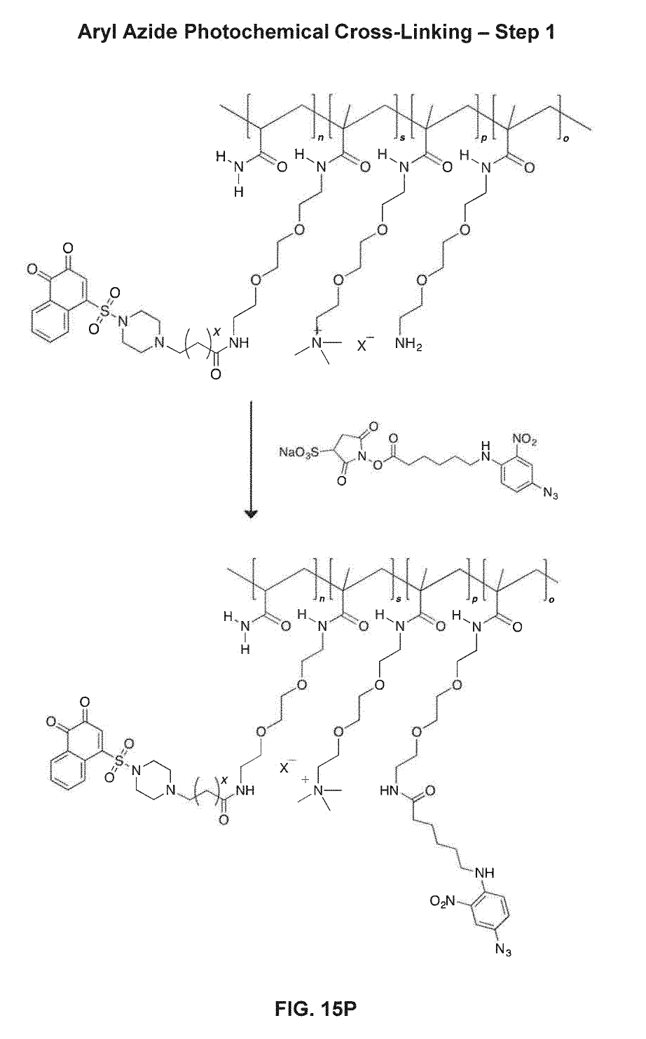

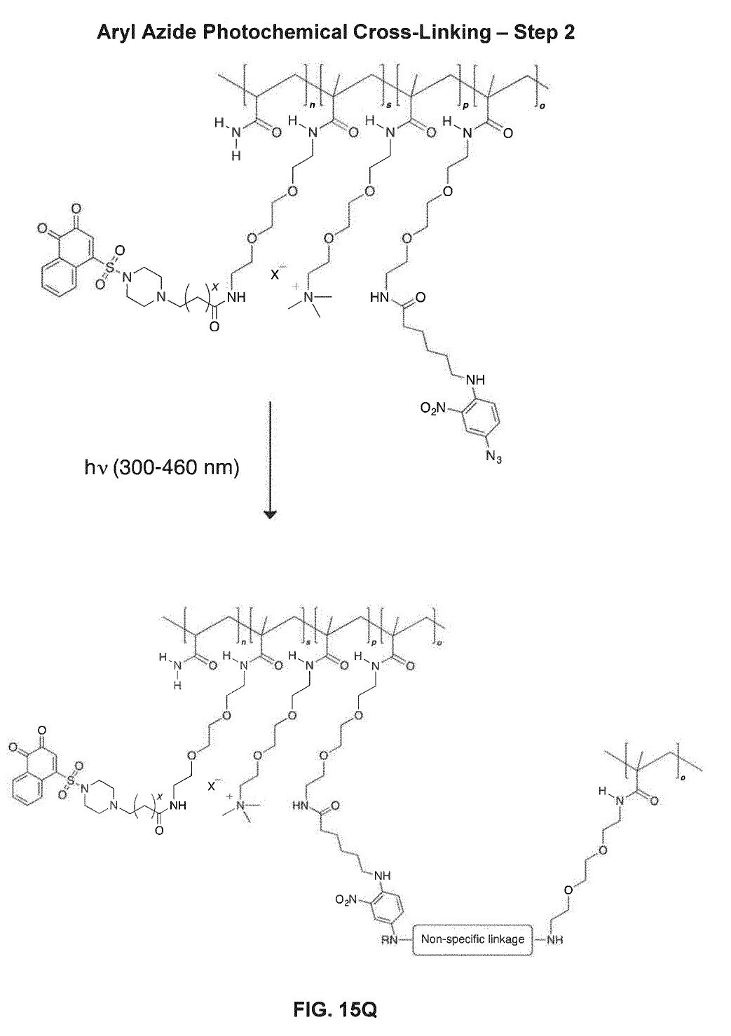

The functional hydrogel materials of the present invention include cross-linked copolymer networks prepared from acrylamide, methacrylamide, acrylate, and/or methacrylate co-monomers equipped with functional groups either alone or in combination with non-functionalized acrylamide, methacrylamide, acrylate, and/or methacrylate co-monomers that ultimately afford mechanisms to immobilize enzyme components, install tethered redox species, and tune a range of hydrogel properties. The resulting hydrophilic polymer backbones can be equipped with pendants bearing terminal amine and ammonium functional groups (see, e.g., the structure shown in FIG. 13A) and/or linking groups (e.g., those made with monomers as shown in FIGS. 2A-2B). Amine groups are reactive sites for orthogonal chemistries that allow enzyme tethering and installation of functional components such as tethered redox mediators and enzymes (see, e.g., FIGS. 12A-12C, 13C, 13E, 13G, 14A-14B, and 15A-15Q). Cationic ammonium groups participate in favorable electrostatic interactions with enzyme components that prevent phase separation (Heller, A.; Feldman, B. Accounts of Chemical Research 2010, 43, 963-973) and improve enzyme stability. The elongated and flexible pendants facilitate favorable reactions and interactions by endowing amine and ammonium groups with increased amplitude of motion relative to such groups that are confined in close proximity to polymer backbones of the hydrogel network. Hydrogels of the invention can be prepared from solutions of monomers, cross-linker, enzyme, and photoinitiator in aqueous phosphate buffer (pH 7.4) by free radical polymerization using ultraviolet or visible light photocuring techniques (chemical curing, electropolymerization and other techniques are also viable). The hydrogel forming solutions generally have overall monomer concentrations of 10-30% (w/v), monomer mass ratios of 8:1:1 (acrylamide and/or methacrylamide and/or acrylate and/or acrylamide:amine monomer:cationic monomer), cross-linker concentrations of 1-5% (w/v), and enzyme concentrations of 0.4-2.0 mg/mL. Exemplary amine monomers are described above in Example 1 and shown in FIGS. 1A, 1B, 3A, 3B, 3D, and 3E. Exemplary cationic monomers are described above in Example 1 and shown in FIGS. 1C and 3C. Exemplary monomers “pre-functionalized” with one of two members of corresponding linking groups are shown in FIGS. 2A and 2B. Acrylate and methacrylate monomers corresponding to the acrylamide and methacrylamide monomers are encompassed by FIGS. 1A-1C and can be generated using procedures similar to those described above in Example 1. Any acrylamide-based monomer described herein can be replaced with a corresponding acrylate-based monomer. Exemplary cross-linking monomers are shown in FIGS. 1D, 3F, and 3G. Water soluble photoinitiators employed for preparation of hydrogel materials include bis(mesitoyl)phosphinic acid sodium salt (Grutzmacher, H.; et al. Macromol. Rapid Commun. 2015, 36, 553-557) and 4,4′-azobis(4-cyanovaleric acid) (commercially available) at low millimolar concentrations typically in the range of 0.5-5 mM. To complete hydrogel preparation, further modifications (via orthogonal chemistries) can be performed post-polymerization (see e.g., FIGS. 15A-15Q) in order to tether enzymes or electron shuttles to the polymer backbones as well as cross-link the polymer backbones. Modifying the monomers pre-polymerization is also encompassed by the invention. Enzyme tethering, redox mediator tethering, and polymer cross-linking can be performed in any of a number of different orders. See FIGS. 12A-12C. However, it is preferred that enzyme and electron shuttle tethering occurs prior to cross-linking.

Tethering of enzymes to the hydrogel network, e.g., for immobilization and to prevent leaching, can be achieved by the coupling of reactive amine sites along the polymer backbones with amine- (e.g., lysine) or thiol- (e.g., cysteine) bearing residues of the enzyme by treatment with bis- or multifunctionalized tethering agents as described herein. These include glutaraldehyde (Fernandez-Lafuente, R.; et al. RSC Adv. 2014, 4, 1583), N-hydroxysuccinimide-maleimide (Yu, J.; et al. Microchim Acta 2016, 183, 1-19) and epoxide-bearing linkers (e.g., diglycidyl linkers), among others. Exemplary tethering agents for enzyme tethering are shown in FIGS. 9A-9D and elsewhere herein. Schematic illustrating enzyme tethering are shown in FIGS. 12A-12C.

Analogous chemistries involving pendant amine groups can be employed to install tethered electron shuttles for versions of the invention employing redox polymer hydrogels (see, e.g., FIGS. 15A, 15E, 15F, 15L). Electron shuttle redox species bearing carbonyl, epoxide, N-hydroxysuccinimide, and a variety of other functional groups subject to nucleophilic attack by amines (or other types of coupling reactions) result in covalent coupling and access a variety of redox hydrogels. Exemplary electron shuttles that can be tethered within the hydrogel networks are shown in FIGS. 5A-5I (depicted without reactive functional groups used for installation), 6-8 (depicted with reactive functional groups used for installation), 15B, and 15H. For example, the hydrogel structure shown in FIG. 13A reacts with the epoxide functionalized ferrocene shown in FIG. 3B to form the redox hydrogel shown in FIG. 3C.

An exemplary enzyme that can be immobilized within the hydrogel networks is glucose oxidase (GOx), which can act as an oxidoreductase component. However, many other proteins or enzyme catalysts can be immobilized within the hydrogel networks. Versions of the invention disclosed herein are designed to be most compatible with water-soluble enzymes such as GOx that are known to be polyanions at physiological pH (pH 7.4). Compatibility is achieved via favorable electrostatic interactions between the cationic pendants decorating the polymer backbones and the polyanionic enzyme. However, these materials can serve as effective scaffolds to immobilize polycationic and neutral enzymes as well.

Preparation of the functional hydrogels can be performed by first polymerizing monomer building blocks in the absence of cross-linking agents to yield soluble linear copolymer. The soluble linear polymer can be subsequently functionalized and cross-linked to form hydrogels.

Formation of the enzymatic redox hydrogel post-polymerization can include each of three distinct processes: (1) Covalent tethering of electron shuttle species to polymer through pendant functional groups; (2) Covalent tethering of enzyme to polymer through pendant functional groups (ultimately resulting in immobilized enzyme); and (3) Covalently cross-linking polymer chains into a polymer redox hydrogel equipped with tethered electron shuttle and immobilized enzyme. These steps can be performed simultaneously (FIG. 12A, panel C, and FIG. 12B), or separately (e.g., sequentially) (FIG. 12C). If performed separately, the steps can be performed in any order. However, it preferred that electron shuttle tethering and enzyme tethering precede cross-linking. In various versions in which electron shuttle tethering and enzyme tethering precede cross-linking, electron shuttle tethering can precede enzyme tethering, enzyme tethering can precede electron shuttle tethering, or electron shuttle tethering and enzyme tethering can occur simultaneously.

These three distinct processes (i.e., electron shuttle tethering, enzyme tethering, and cross-linking) can be achieved using a single chemistry, such as an embodiment of the invention in which a homobifunctional amine-reactive linker is employed to tether an amine-bearing electron shuttle, immobilize an enzyme through amine-bearing residues, and form cross-links to/between polymer chains equipped with pendant amine functional groups, or by using multiple, different orthogonal chemistries. An advantage of achieving all three processes using a single chemistry is convenience, simplicity, and fewer synthetic steps. A disadvantage of using a single chemistry for all three processes is the inability to precisely control each process independently of the others. Employing multiple orthogonal chemistries constitutes a means to control one or more of the processes while the others remain inert. For example, by employing different orthogonal chemistries for electron shuttle tethering and cross-linking, both electron shuttle loading and degree of cross-linking can be precisely controlled, allowing for tuning and access to materials otherwise unattainable through a “single chemistry'” approach.

Examples of functionalizing the polymers post-polymerization with different orthogonal chemistries are shown in FIGS. 15A-15Q. These examples illustrate multiple orthogonal chemistry based strategies to independently install electron shuttle and cross-link polymer chains to afford redox hydrogels.

In one version, an amine-bearing linear copolymer is equipped with a tethered electron shuttle via NHS-ester:amine coupling Chem. Soc. Rev., 2009, 38, 606-631) to yield an electron shuttle-bearing copolymer (FIG. 15A). A second NHS-ester:amine coupling between the electron shuttle-bearing copolymer and a tetrazole compound (Angew. Chem. Int. Ed. 2008, 47, 2832-2835) forms a tetrazole-bearing copolymer (FIG. 15C). When combined with bifunctional alkene-bearing linkers (alkene=alkene, acrylate, acrylamide, methacrylate, methacrylamide, vinyl), the tetrazole-bearing copolymer undergoes tetrazole photo-click based cross-linking (Angew. Chem. Int. Ed. 2008, 47, 2832-2835; Top Curr Chem (J). 2016 February; 374(1)) upon treatment with UV radiation to form a cross-linked redox hydrogel (FIG. 15D).

In another version, an amine-bearing linear copolymer is first equipped with tethered electron shuttle via copper-free click chemistry. This occurs by first installing azide functional groups (FIG. 15E) followed by treatment with electron shuttles bound to cyclooctyne groups such as DIMAC (Ellen M. Sletten and Carolyn R. Bertozzi. Org. Lett., 2008, 10(14):3097-3099) or ALO (Pamela V. Chang, Jennifer A. Prescher, Ellen M. Sletten, Jeremy M. Baskin, Isaac A. Miller, Nicholas J. Agard, Anderson Lo, and Carolyn R. Bertozzi. PNAS, February 2, 2010, vol. 107, no. 5, 1821-1826) (FIG. 15F) to yield electron shuttle-bearing copolymer. Shuttle loading via copper free click chemistry affords favorable features such as good selectivity and yield of coupling steps that allow a high degree of control of the degree of shuttle loading (Angew. Chem. Int. Ed. 2009, 48, 6974-6998). Analogously to the cross-linking route shown in FIGS. 15C and 15D, the electron shuttle-bearing copolymer of FIG. 15F can be equipped with tetrazole groups (FIG. 15I) and UV treated to induce tetrazole photo-click cross-linking (FIG. 15J) to yield a hydrogel structure. Cross-linking can also occur using on epoxide linkers (FIG. 15K).

In another version, an amine-bearing linear copolymer undergoes carbodiimide driven coupling of a carboxylic acid-bearing electron shuttle with its pendant amine groups to yield electron shuttle-bearing copolymer (FIG. 15L). The electron shuttle-bearing copolymer can then undergo chemical cross-linking using NHS-ester bifunctional linkers (FIG. 15M). Alternatively, commercially available NHS-ester functionalized diazirine and aryl azide compounds can be used for non-specific photochemical cross-linking with treatment with UV and near-UV radiation (Thermo Scientific Crosslinking Technical Handbook (tools.thermofisher.com/content/sfs/brochures/1602163-Crosslinking-Reagents-Handbook.pdf)) (FIG. 15N, 105, 105, and 105). The cross-linking occurs by reactions of carbenes and nitrenes formed upon irradiation of diazirines and aryl azides, respectively (Thermo Scientific Crosslinking Technical Handbook, (tools.thermofisher.com/content/sfs/brochures/1602163-Crosslinking-Reagents-Handbook.pdf)) (Angew. Chem. Int. Ed. 2009, 48, 6974-6998).

Other orthogonal chemistry based synthetic routes are accessible via combinations of common and well known orthogonal chemistries (ACS Chem. Biol. 2014, 9, 592-605).

The electron shuttle analogs bearing complimentary linking groups used in the reactions of FIGS. 15A, 10F, and 10L can be readily accessed using well-defined bioconjugation tools. The electron shuttles used in the reactions of FIGS. 15A and 15L can be prepared as shown in FIG. 15B using carbodiimide conjugation chemistry. Electron shuttles bearing DIMAC and ALO cyclooctyne functional groups used in the reaction of FIG. 15F can be prepared using carbodiimide-driven carboxylic acid:amine coupling (FIGS. 15G and 15H). It is important to note that DIMAC and ALO are among the most water soluble of the cyclooctane compounds known to be effective copper free click coupling partners with azides and are therefore well suited for use in this system.

The use of photoinduced cross-linking facilitates various solution processing strategies. Homogeneous hydrogel networks are readily accessed after solution processing via photocuring using wavelengths in the range of 254-350 nm UV (Polym. Chem., 2014, 5, 2187-2201). Chemical cross-linking is also useful but can be more prone to inhomogeneity with respect to the spatial distribution of cross-links (caused by uneven mixing).

Enzyme immobilization steps can either be adapted or directly employed using the methods of FIGS. 15A-15Q and others.

In some versions of the invention, monomer building blocks bearing different, non-amine terminal reactive functional groups (e.g., linking groups) (FIGS. 2A-2B) are copolymerized to yielding soluble linear copolymer. The different reactive functional groups facilitate independent cross-linking, installation of tethered electron shuttle, and/or immobilization of enzyme. These monomers can be polymerized with or without the monomers bearing terminal amines (e.g., FIGS. 1A-1C and FIGS. 3A-3E).

The hydrogels of the invention serve several key purposes critical for use in sensing applications. In addition to providing a scaffold for installing and confining functional components, the hydrogels provide the abilities to tune and control mass transport of analytes within the hydrogel (such as concentration dependent glucose flux), to regulate the diffusion properties of participating water-soluble species, to regulate pH, and to stabilize enzyme components. By modulating variables such as monomer and cross-linker structure, co-monomer ratios, cross-linker ratio, and curing conditions, properties including mechanical strength, water content, pore size, and swelling/deswelling characteristics can be tuned. Cross-linker structure, namely the length and rigidity of the linker chain, as well as cross-linker mass ratio heavily influences pore size, which in turn influences mass transport factors such as analyte flux and the diffusion rates of ions and relevant water-soluble compounds within the hydrogels. Pore size also determines the degree of intermolecular interactions that govern (in part) enzyme stabilization. Within the context of sensing applications, the ability to tune and exploit mass transport properties within hydrogels, specifically the regulation of analyte flux into the hydrogel, advantageously renders concentration-dependent analyte flux into the hydrogel the dominant factor governing electrochemical sensor response rather than the less stable activity of catalyst or electrocatalyst components (Heller, A. Annu. Rev. Biomed. Eng. 1999, 1, 153-175). Such systems yield improved stability of the sensor response, which is advantageous considering the ever decreasing sample sizes of state of the art biosensor systems.

The multifunctional hydrogel materials disclosed herein address the materials-based needs of next generation electrochemical enzymatic sensing systems. Not only do the functional materials described herein meet the general safety and accuracy requirements for commercial electrochemical enzymatic sensing systems, but they also address the challenges associated with sensing using very small sample volumes.

Advantageous features of the hydrogel materials of the invention and hydrogels made therefrom can be summarized as follows: 1) The versatile hydrogel materials can be functionalized to facilitate a wide range of sensing mechanisms which makes them well suited to help minimize both the background signal caused by interferants and the susceptibility to sample-to-sample variances (such as variances in ambient oxygen concentration that can be challenging to address in glucose sensing systems); 2) The tunability of the hydrogel system allows optimization of the stability and accuracy of sensor response through control of mass transport properties such as analyte flux and the diffusion rates of relevant dissolved species such as charge balancing ions; 3) The combination of tunability and the presence of sites for orthogonal chemistry makes the hydrogels amenable to implementation of sensor amplification strategies such as redox cycling or indirect detection schemes (such as glucose detection based on differential oxygen); 4) The materials afford a wide range of processing options granting the flexibility required to optimize factors such as electrode configuration and surface area for maximum sensor efficiency and sensitivity; and 5) The system is generally versatile enough to address the majority of challenges associated with development of next generation sensing technologies.

### Example 4. Functionalized Hydrogel-Based Electrochemical Glucose Sensing

This example discusses an electrochemical glucose detection system based on functional hydrogel materials containing immobilized oxidoreductase enzymes, including those described in Example 3.

The general detection system architecture of the electrochemical glucose detection system includes working, counter, and reference electrodes functionalized with the enzyme-containing hydrogel material. Sensing is based upon direct or indirect measurement of electrons from enzymatic glucose oxidation. Signal transduction, in the form of an electrochemical response that is directly correlated with glucose concentration, is facilitated by the hydrogel material, which serves to immobilize the enzyme component (e.g., in close proximity of the electrodes), regulate mass transport rates of analytes (and other dissolved compounds that participate in device function), and, in redox hydrogel versions of the invention, mediate transport of electrons from the reduced enzyme to working electrodes. FIGS. 16A and 16B show schematic representations of glucose sensing via direct hydrogen peroxide detection (FIG. 16A) and electron shuttle-based detection (FIG. 16B).

As described in Example 3, the hydrogel materials combine key features of both solids and liquids, are readily tunable, afford stabilizing effects for biological enzyme catalyst components, can be readily formed in aqueous conditions compatible with enzyme components (i.e. that preserve enzyme activity), and are amenable to a wide range of processing methods. The ability to functionalize the hydrogel materials for device operation based on multiple detection modes affords the ability to tune operating voltages, maximize sensitivity, and minimize background signal. The ability to tune and exploit mass transport properties within the hydrogels, specifically the regulation of glucose flux into the hydrogel, generates a system in which concentration-dependent glucose flux into the hydrogel is the dominant factor governing electrochemical response rather than the less-stable activity of catalyst or electrocatalyst components. The resulting glucose sensing electrodes are capable of glucose detection using sample volumes in the nanoliter/picoliter range, which is well below the present state of the art.

An exemplary oxidoreductase component of the detection system is glucose oxidase (GOx). In a two electron process the oxidized form of GOx selectively binds and oxidizes glucose to produce gluconolactone and the reduced form of GOx. The reduced form of GOx can then undergo redox reactions with either oxygen to form hydrogen peroxide or with a suitable electron shuttle redox species, in both cases regenerating the oxidized form of GOx and transferring electrons from glucose oxidation to carriers that facilitate sensor signal transduction in the form of an electrochemical response.

The electrodes can be composed of a variety and/or combination of conductive materials and are preferably high surface area conductive materials. Working and counter electrode materials can be gold, platinum, or conductive carbon (to name a few) while reference electrodes are silver. Platinum electrodes with surfaces composed of platinum nanostructures/nanoparticles afford high electroactive surface area resulting in the increases in signal (relative to smooth platinum electrodes) for sensing using small sample volumes. Nanostructured platinum electrodes are prepared from platinum electrodes using platinum electrodeposition methods. Electrodeposition of platinum nanostructures is achieved by a modified version of a reported procedure (Burke, J. J.; Buratto, S. K. J. Phys. Chem. C 2013, 117, 18957-18966) in which platinum electrodes, submerged in a plating solution composed of 5 mM chloroplatinic acid (providing superior results to the platinic acid used by referenced method of Burke et al.) in 1 M sulfuric acid (aq.) and configured as working electrodes in 3-electrode cells with platinum counter and silver-silver chloride reference electrodes, are subjected to square wave pulsed potential deposition cycles with low and high potentials of −1.0 V and 0.5 V, respectively, at a frequency of 167 Hz for 900 seconds using a 50% duty cycle. This deposition method results in, for example, ˜60-fold increases in electrochemical surface area of electrodes covering a ˜1 mm² area of a glass substrate relative to that prior to electrodeposition. The large increases in electrochemical surface area afforded by the electrodeposition process is important for sensing using small sample volumes.

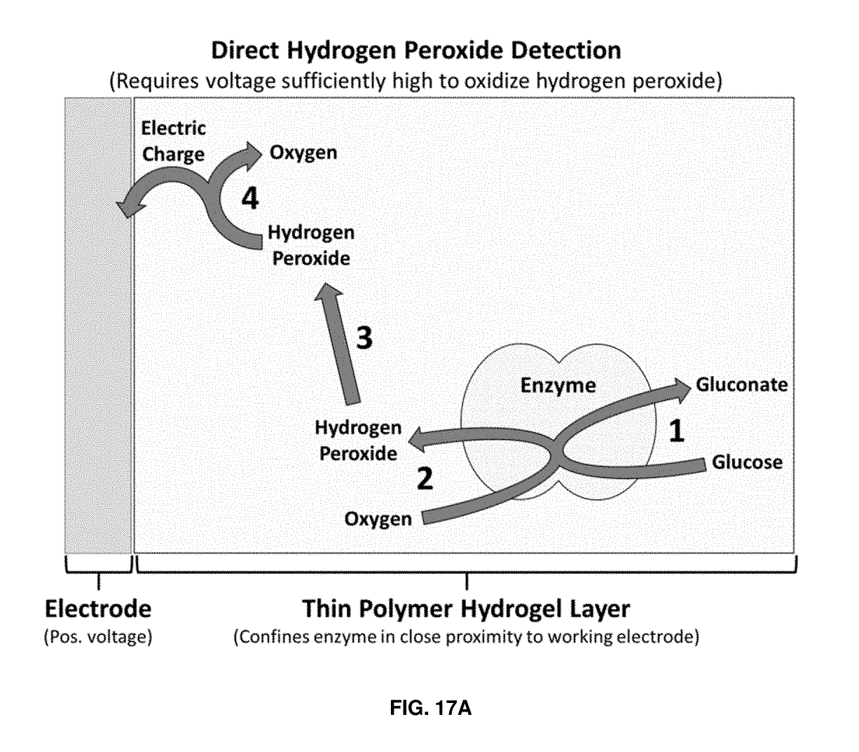

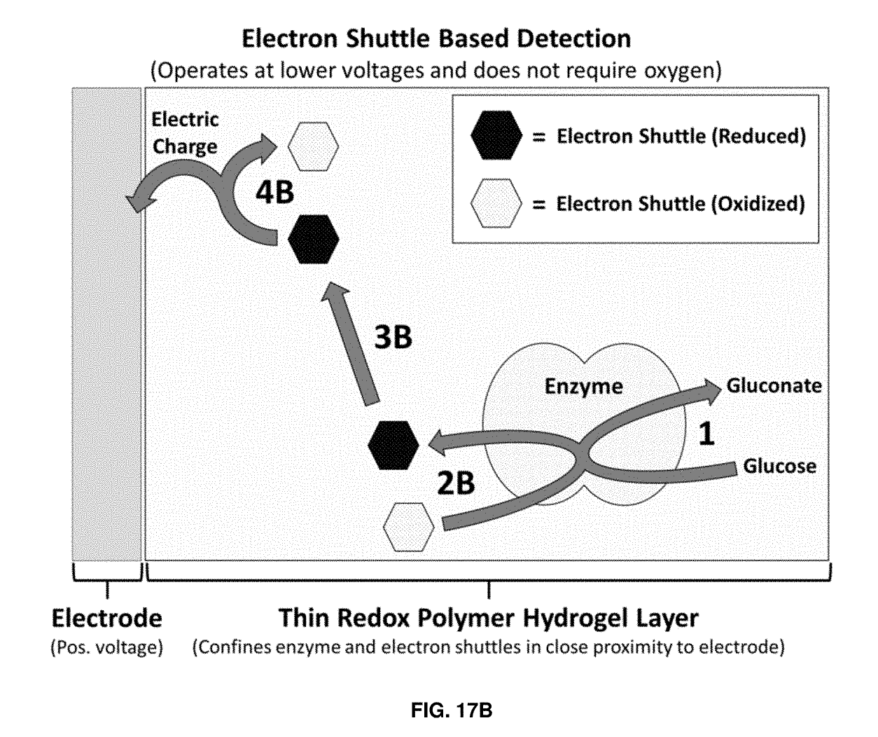

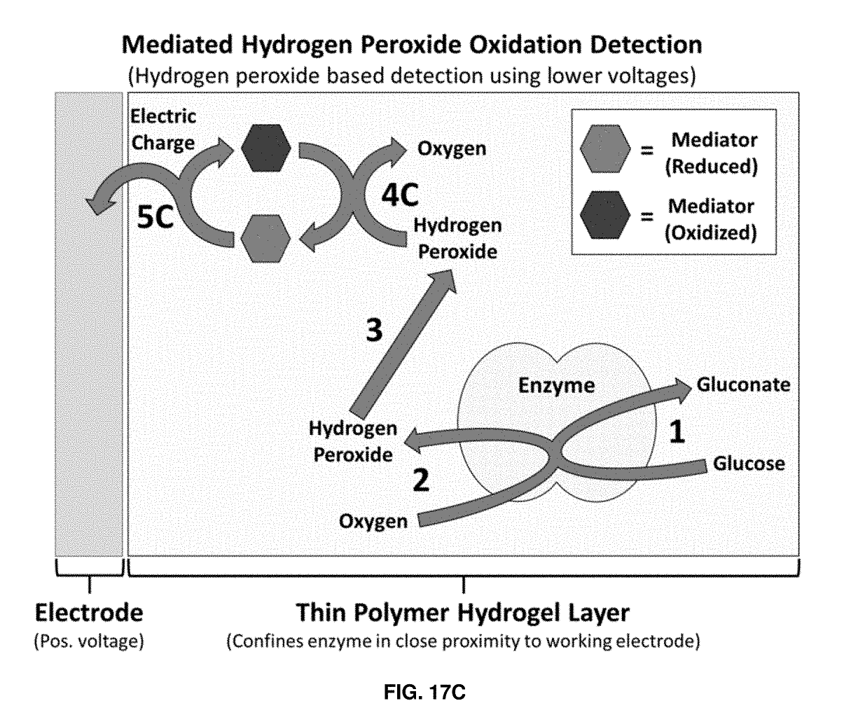

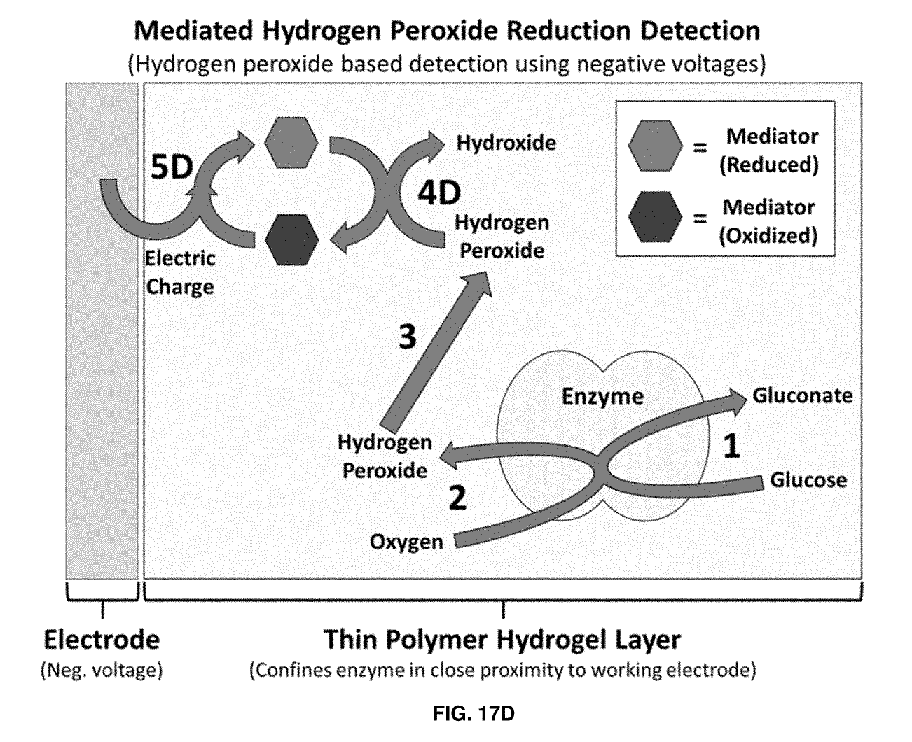

The ability to readily modify the hydrogel materials facilitates use of multiple detection modes, including direct electrochemical hydrogen peroxide detection, electron shuttle-based detection via redox species, and mediated hydrogen peroxide detection (based on hydrogen peroxide oxidation or reduction). FIGS. 17A-17D are schematic cartoons depicting a device operating in each of these detection modes.

In direct hydrogen peroxide detection mode (FIG. 17A), the reduced form of GOx resulting from glucose oxidation (FIG. 17A, 1) reduces oxygen (FIG. 17A, 2) to form hydrogen peroxide (FIG. 17A, 3) that then transports those electrons via diffusion to the working electrode where they can be collected via the electrochemical oxidation of hydrogen peroxide (FIG. 17A, 4). The operating potential of this detection mode is defined by the potential at which electrochemical hydrogen peroxide oxidation occurs and is in the range of ˜400-700+ mV vs Ag/AgCl.

The electron shuttle detection mode (FIG. 17B) allows device operation at lower potentials as this mode utilizes redox compounds (electron shuttles; e.g., FIGS. 5A-5I, 6, 7A, 7B, and 8) capable of oxidizing the reduced enzyme (FIG. 17B, 2B) (following glucose oxidation (FIG. 17B, 2B)), followed by diffusing of the reduced redox compounds to the working electrode (FIG. 17B, 3B) and oxidation at the working electrode (FIG. 17B, 4B) at lower voltage than electrochemical hydrogen peroxide oxidation. Choice of electron shuttles allows tuning of operating potential for maximizing sensitivity and minimizing the background signal resulting from electrochemical reactions of other oxidizable species in the sample. Electron shuttle-based detection does not require the presence of oxygen.

Mediated hydrogen peroxide oxidation (FIG. 17C) and reduction modes (FIG. 17D) operate in a similar manner as direct hydrogen peroxide detection (FIG. 17A) but can operate at lower positive (mediated hydrogen peroxide oxidation) or low negative (mediated hydrogen peroxide reduction) potentials by employing redox mediator compounds that oxidize (FIG. 17C, 4C) or reduce (FIG. 17D, 4D) hydrogen peroxide followed by their reformation at the working electrode (FIG. 17C, 5C; FIG. 17D, 5D). In these mediated detection modes, the electrochemical response corresponding to reformation the mediator species is proportional to glucose concentration. These mediator species include iron- or cobalt-based materials (FIGS. 18A-18C) including Prussian blue and co-phthalocyanine and can be employed as electrodeposited layers on working electrodes or tethered within the hydrogel network. The various different detection modes of the invention are representative of the versatility provided by the invention for achieving glucose detection using extremely small sample volumes.

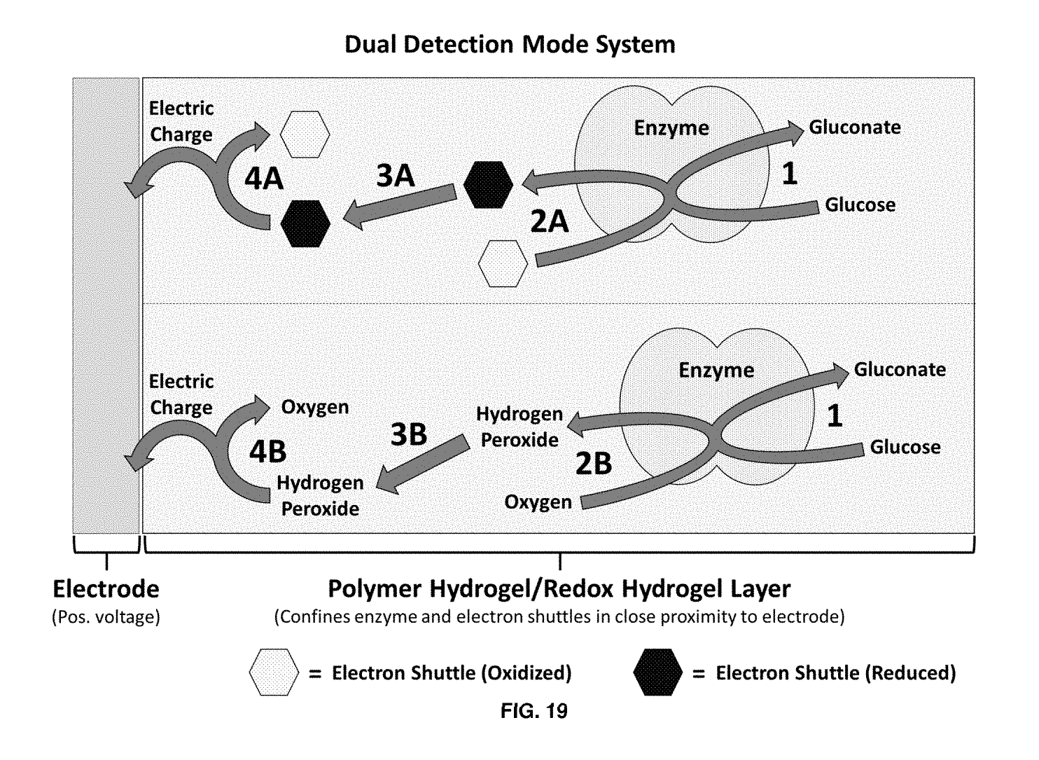

Detection of glucose in blood, interstitial fluid, or other sample media (e.g., tears) can be subject to sample and environmental variations such as differences in oxygen concentration. Certain versions of the present invention allow collection of electrons from enzymatic glucose oxidation independently of oxygen concentration. FIG. 19, for example, is a schematic representation of a dual detection mode version of the invention in which electrons from glucose oxidation are collected by way of both direct hydrogen peroxide oxidation and electron shuttle-based detection. This dual detection mode employs redox polymers with tethered electron shuttles and/or non-redox polymers in hydrogels with untethered electron shuttles that can accept electrons from the reduced enzyme (resulting from glucose oxidation) and then be subsequently oxidized at the working electrode at potentials in a range overlapping that of electrochemical hydrogen peroxide oxidation. As such, electrons from glucose oxidation can be collected regardless of whether or not they participate in oxygen reduction to form hydrogen peroxide or in reduction of the electron shuttles. Thus, by employing electron shuttles with redox potentials in a suitable range, the system can function independently of oxygen concentration as electrons from glucose oxidation can be measured regardless of their transport pathway. This dual detection scheme is less susceptible to variations in environmental conditions (such as temperature, atmospheric pressure, atmospheric composition, etc.) that can influence the concentration of dissolved oxygen.

Some versions of the invention employ redox cycling amplification. Redox cycling amplification amplifies the signal in very small sample volumes by exploiting electrodes and/or sacrificial electron donors for repeatedly replenishing the reduced product of the enzymatic process following initial oxidation of the of the reduced product, e.g. an electron shuttle, at the working electrode. This allows the reduced product to be repeatedly oxidized at the electrode. In this way, the number of electrons collected/measured at the electrode per molecule of analyte is multiplied, resulting in amplified signal. For example, in a general version of this invention that utilizes redox cycling signal amplification, the oxidized form of the enzyme is first reduced upon oxidation of the substrate, the reduced form of the enzyme is then oxidized by a precursor analog of the oxidized form of an electron shuttle (an analog that is not readily reduced by sacrificial donors) to form the reduced form of the electron shuttle, the reduced form of the electron shuttle is then oxidized at the electrode to form the oxidized form of the electron shuttle that can then undergo successive redox cycles of reduction by sacrificial electron donors followed by oxidation at the electrode. Thus, for each electron shuttle reduced by the enzyme, the electrons originating from the substrate as well as the sacrificial donors can be collected, thereby amplifying the signal. Such an approach permits collecting 2-20 fold the electrons per glucose molecule that is oxidized by GOx instead of just two.

Some versions of the invention employ indirect glucose sensing based on differential oxygen. In such a system, the oxygen concentration is measured initially and as a function of time as sample glucose is oxidized and oxygen is consumed in the process. Oxygen can be measured electrochemically in a very similar manner as pH (except that instead of the electrode being selective for protons it is selective for oxygen).

The hydrogel-based electrochemical glucose sensing system disclosed herein exploits the versatility and tunability of custom multifunctional hydrogel materials to redefine the threshold for glucose sensing sample volumes. This system meets the general safety and accuracy requirements for commercial electrochemical enzymatic glucose sensing systems and addresses the challenges associated with sensing using very small sample volumes.

The features of the hydrogel-based electrochemical glucose sensing system disclosed herein can be summarized as follows: 1) The versatile hydrogel materials can be functionalized for device operation in multiple and even simultaneous detection modes, allowing background signal caused by interferants and susceptibility to variances in ambient oxygen concentration to be minimized; 2) The tunability of the hydrogel system permits optimizing the stability and accuracy of sensor response through control of mass transport properties such as glucose flux and the diffusion rates of relevant dissolved species such as charge balancing ions; 3) The combination of tunability and the presence of sites for orthogonal chemistry allows for the implementation of amplification strategies such as redox cycling or alternative detection modes such as those based on differential oxygen; 4) The materials afford a wide range of processing options granting the flexibility required to optimize factors such as electrode configuration and surface area for maximum sensor efficiency and sensitivity; 5) The system is versatile enough to address the majority of challenges associated with sensing using very small sample volumes; and 6) high surface area platinum electrodes with platinum nanostructure surfaces afford high electrochemical surface areas for sensing with small sample volumes and do not limit device sensitivity and overall performance.

### Example 5. Electron Shuttle-Based Glucose Sensing

Preparation of tethered electron shuttle redox hydrogel sensors and electrodes based on single-step shuttle tethering, enzyme immobilization, and cross-linking. Linear copolymer was prepared by first combining monomer stock solutions with 30% (w/v) concentrations in 100 mM PB buffer (pH 7.4) to the desired weight ratio (ex: 3:7:1, AA:MeAA-PEG-NH₂:MeAA-PEG-NMe₃) (AA=acrylamide; MeAA=methacrylamide; PEG=polyethylene glycol), diluting with buffer to a final concentration of 10% (w/v), and degassing via sparging with nitrogen. Oxygen-free photoinitiator BAPO-Ona (Macromol. Rapid Commun. 2015, 36, 553-557) was added in the form of a 25-mM stock solution in buffer via degassed syringe to a concentration of 0.1-1 mM (0.5 mM in most cases), and the solution was stirred under inert atmosphere while irradiated at 405 nm using a Dymax Bluewave QX4 spot curing system equipped with a 405 nm VisiCure LED wand (50 cycles; cycle=30 sec at 100% power then 10 sec at 0% power). The resulting linear copolymer solution was used as-is for preparation of hydrogels.

Redox hydrogels were prepared and installed onto device substrates by first weighing low-mg quantities (˜5-15 mg) of the NQSA-2 electron shuttle (see Example 2) into a 1.5-mL vial. Linear copolymer solution (10% w/v) was added in volumes such that the desired mole ratio of amine-bearing pendant groups to shuttle was achieved, and the resulting mixture was stirred for 3-5 minutes before glucose oxidase (GOx) enzyme (Calzyme) was added in the form of a 20-mg/mL stock solution in 100-mM PB buffer (pH 7.4) to final concentrations of 1-5 mg/mL. The resulting mixture was stirred for 1-2 minutes. The homobifunctional cross-linker sebacic acid bis(N-succinimidyl) ester was added to the desired mole ratio (relative to amine bearing pendant groups) in the form of a fine powder with stirring. The resulting mixture quickly increased in viscosity as the reactions proceeded and was deposited onto device substrates (microneedles or chips with high surface area electrodes by drop casting, blade coating, or dip coating, depending upon the substrate. The hydrogels were then allowed to cure overnight in the dark at high humidity. Once cured, the devices were soaked in buffer for several hours to swell the hydrogels and wash away leachables. The gels were then allowed to dry prior to installation of any glucose flux regulating membranes deposited via solution processing.

Solution-processed membranes were used for kinetic control (e.g., regulating glucose flux). The membranes were typically composed of copolymers of polyurethane-linked polyethylene glycol (PEG) (Mₙ=200-400) and polydimethylsiloxane (PDMS) (Mₙ=2500) blocks (e.g., 30:70; PEG:PDMS mole percent ratio). These materials and their preparation can be found in the patent literature (U.S. Pat. Nos. 5,777,060 and 5,882,494). Membranes were installed via drop casting, dip coating, or spray coating from 1-4% (w/w) solutions in THF, 1,4-dioxane, or ethanol and allowed to dry in air at room temperature for 10-20 min. Devices were then soaked in buffer for 24 hours, over which time membranes wet and gels re-swelled.

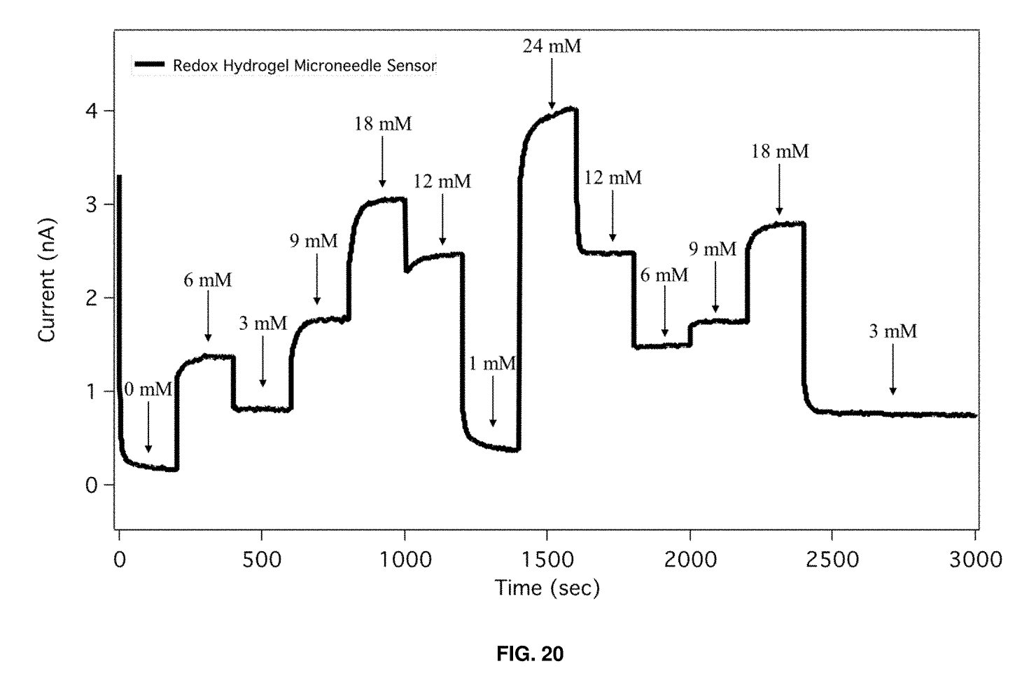

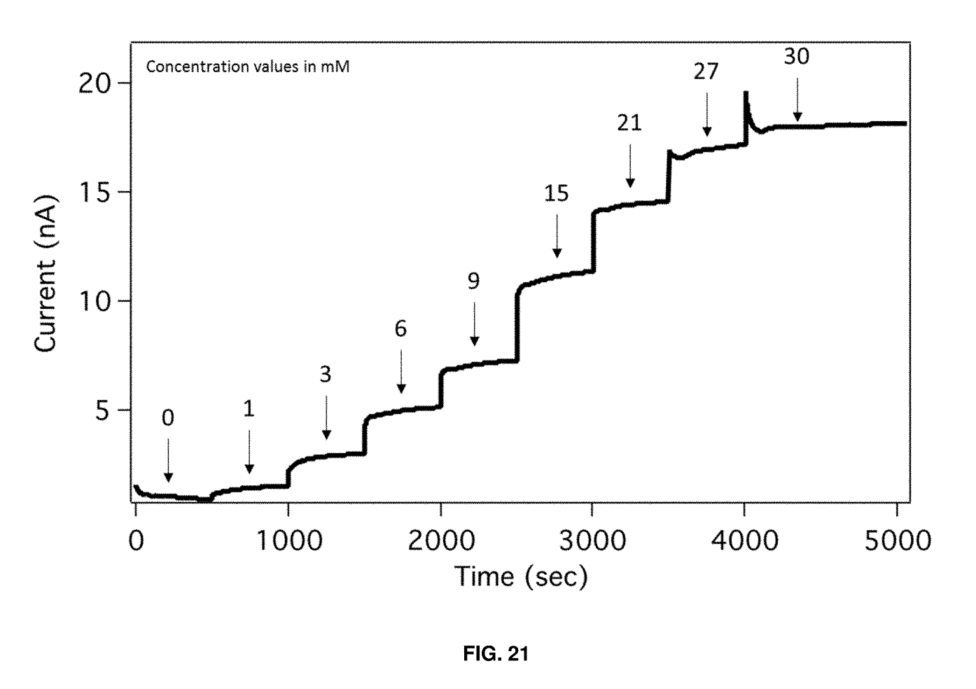

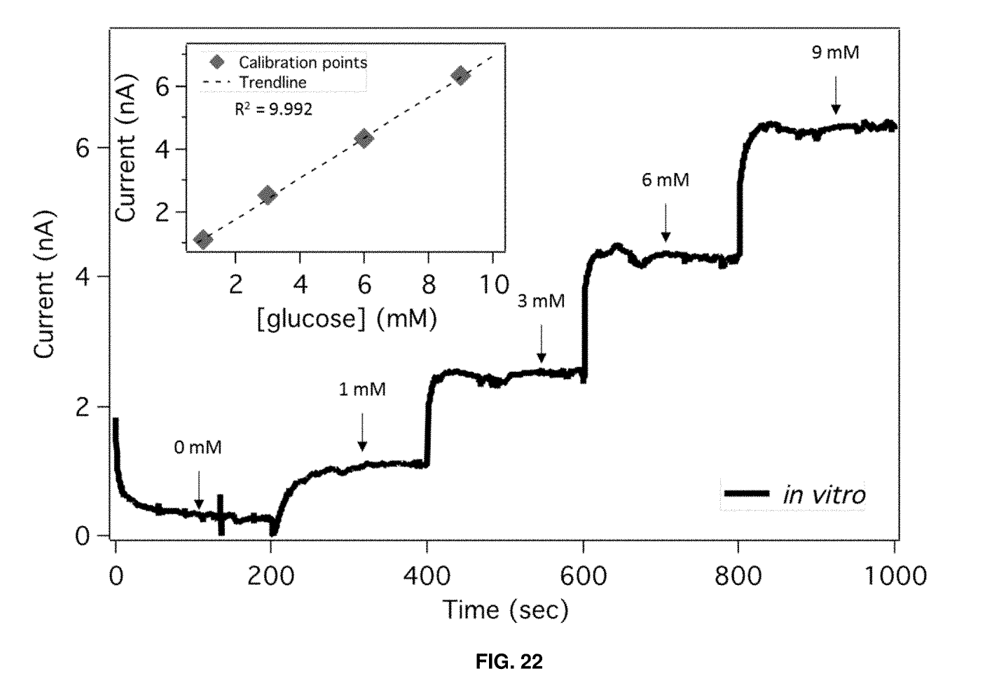

Electrochemical glucose sensing with redox hydrogel-based sensors. Redox hydrogel-based electrochemical glucose sensors included either custom microneedles or glass chips with two high-surface-area platinum electrodes in contact with a layer of redox hydrogel and a polymer membrane top coating. Sensors were evaluated/operated in 2-electrode configuration i-t mode with sensing (working) electrodes poised at 50 mV relative to the counter electrode. The employed sample solution/electrolyte was either 100 mM PB buffer (pH 7.4) or 50 mM PBS containing 154 mM NaCl (pH 7.4). Sample volumes ranged from 50 μL (for chips only, sample in the form of droplet placed atop the electrode area) to 2-10 mL (microneedles with tips submerged into sample solution). Data shown here corresponds to sensors operated at ambient temperature in air. Glucose concentrations were varied randomly or linearly and sensors were typically operated for time intervals of 150-1000 sec for each glucose concentration, at which time data collection was paused and the sample solution was swapped for the next glucose concentration before resuming data collection. A typical sample swap took no longer than 30-60 sec. Results are shown in FIGS. 20-22.

### Example 6. Peroxide-Based Glucose Sensing

Preparation of GOx hydrogels for peroxide detection mode glucose sensing. Hydrogel forming solution was prepared by combining monomer stock solutions with 30% (w/v) concentrations in 100-mM PB buffer (pH 7.4) to the desired weight ratio (ex: 7:3:1, AA:MeAA-PEG-NH₂:MeAA-PEG-NMe₃), adding cross-linker (tetra(ethylene glycol) diacrylate) to desired weight fraction (1-2% w/v; added as pure material) and GOx enzyme to the desired concentration (0.4-3.0 mg/mL; added in the form of a 10-20 mg/mL stock solution in buffer), diluting with buffer to a final concentration of 10% (w/v), and degassing via sparging with nitrogen. Oxygen-free photoinitiator, BAPO-Ona (Macromol. Rapid Commun. 2015, 36, 553-557), was added in the form of a 25 mM stock solution in buffer via degassed syringe to a concentration of 0.1-1 mM (0.5 mM in most cases), and the solution was mixed under inert atmosphere, cast onto device substrates in contact with electrodes via dip coating, drop casting, or blade coating, and irradiated at 405 nm using a Dymax Bluewave QX4 spot curing system equipped with a 405 nm VisiCure LED wand (10 cycles; cycle=30 sec at 100% power then 10 sec at 0% power). Once cured, the devices were soaked in buffer for several hours to swell the hydrogels and wash away leachables. The gels were then allowed to dry prior to installation of any glucose flux-regulating membranes deposited via solution processing. Data shown here corresponds to a Pt wire coated with GOx hydrogel without any flux regulating membrane layer.

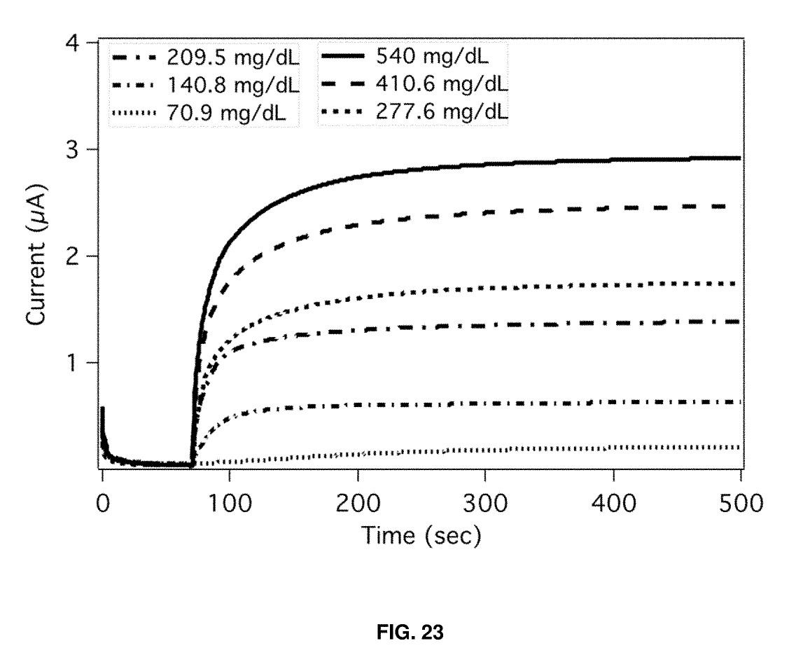

Electrochemical glucose sensing with peroxide detection-based sensor. GOx hydrogel-based electrochemical glucose sensors included either dip-coated Pt wire sensing electrodes, custom microneedles, or glass chips with two high-surface-area platinum electrodes in contact with a layer of GOx hydrogel with or without a polymer membrane top coating. Sensors were evaluated/operated in 2- or 3-electrode configuration i-t mode with sensing (working) electrodes poised at 600 mV relative to the counter electrode. The employed sample solution/electrolyte was either 100 mM PB buffer (pH 7.4) or 50 mM PBS containing 154 mM NaCl (pH 7.4). Sample volumes ranged from 50 μL (for chips only, sample in the form of droplet placed atop the electrode area) to 2-10 mL (microneedles with tips submerged into sample solution). Data shown here corresponds to sensors operated at ambient temperature in air. Glucose concentrations were varied randomly or linearly and sensors were typically operated for time intervals of 150-1000 sec for each glucose concentration, at which time data collection was paused and the sample solution swapped for the next glucose concentration before resuming data collection. A typical sample swap took no longer than 30-60 sec. Alternatively, individual i-t traces were collected for each glucose concentration. Results are shown in FIG. 23.

What is claimed is:

1. A system comprising a polymer, wherein the polymer includes subunits of Formula I: 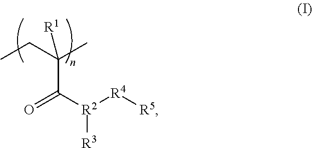
wherein:
R¹ in each instance is independently H, alkyl, cycloalkyl, aryl, heterocycle, heteroaryl, or a combination thereof;
R² in each instance is independently N or O;
R³ in each instance is independently:
H; alkyl, cycloalkyl, aryl, heterocycle, heteroaryl, or a combination thereof; or a nitrogen protecting group when R² is N; or
absent when R² is O;
R⁴ in each instance is a spacer arm;
R⁵ in each instance is Formula II or Formula III: 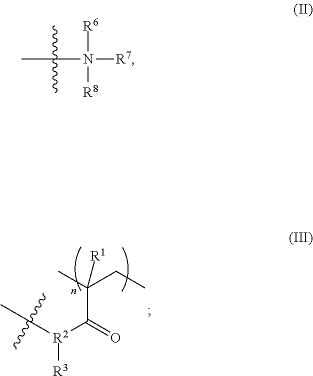
R⁶, R⁷, and R⁸ in each instance are independently H; alkyl, cycloalkyl, aryl, heterocycle, heteroaryl, or a combination thereof; a nitrogen protecting group; a tethered polypeptide; a tethered redox mediator; a linking arm; or a tethered subunit of Formula I, with the proviso that at least one of R⁶, R⁷, and R⁸ may be absent; and
n in each instance is independently a positive integer.

2. The system of claim 1, wherein R⁵ in at least one subunit of the polymer is Formula II, R⁸ is a tethered polypeptide, and the polypeptide is an oxidoreductase.

3. The system of claim 1, wherein R⁵ in at least one subunit of the polymer is Formula II, R⁸ is a tethered polypeptide, and the polypeptide is a glucose oxidase.

4. The system of claim 1, wherein R⁵ in at least one subunit of the polymer is Formula II, R⁸ is a tethered redox mediator, and the redox mediator comprises a compound of Formula VI: 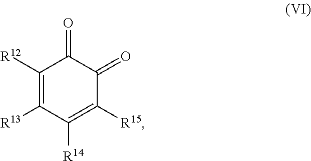
wherein:
R¹² and R¹³ are each independently H, alkyl, cycloalkyl, aryl, heterocycle, heteroaryl, or a combination thereof; or are fused in an aromatic or non-aromatic ring;
R¹⁴ is an electron withdrawing group;
R¹⁵ is H, alkyl, cycloalkyl, aryl, heterocycle, heteroaryl, or a combination thereof; and
the compound of Formula VI is tethered via any one or more of R¹², R¹³, R¹⁴, R¹⁵, and any atom in the aromatic or non-aromatic ring formed by R¹² and R¹³.

5. The system of claim 1, wherein R¹⁴ in the electron shuttle of Formula VI is Formula VII or Formula VIII: 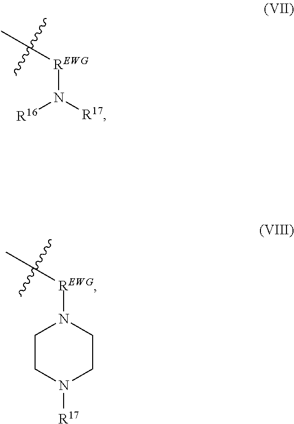
wherein:
R^{EWG} is an electron withdrawing group;
R¹⁶ is H, alkyl, alkyl, cycloalkyl, aryl, heterocycle, heteroaryl, perfluoroalkyl, or a combination thereof; and
R¹⁷ is a tether connected to R⁸.

6. The system of claim 1, wherein R⁵ in at least one subunit of the polymer is Formula II, R⁸ is a tethered redox mediator, and the redox mediator is a 1,2-naphthoquinone.

7. The system of claim 1, wherein R⁵ in at least one subunit of the polymer is Formula II, R⁸ is a tethered redox mediator, and the redox mediator is a 1,2-naphthoquinone comprising an electron withdrawing group connected to a 4-carbon of the 1,2-naphthoquinone.

8. The system of claim 1, wherein R⁵ in at least one subunit of the polymer is Formula II, R⁸ is a tethered redox mediator, the tethered redox mediator is a 1,2-naphthoquinone comprising an electron withdrawing group connected to a 4-carbon of the 1,2-naphthoquinone, and the electron withdrawing group is selected from the group consisting of sulfonate, sulfonamide, ammonium, quaternary ammonium, fluoroaklyl, perfluoroalkyl, nitro, and cyano.

9. The system of claim 1, wherein R⁵ in at least one subunit of the polymer is Formula II, R⁸ is a tethered redox mediator, and the tethered redox mediator is a 1,2-naphthoquinone comprising a sulfonamide connected to a 4-carbon of the 1,2-naphthoquinone.

10. The system of claim 9, wherein the 1,2-naphthoquinone is tethered via a 3-carbon, the 4-carbon, a 5-carbon, a 6-carbon, a 7-carbon, or an 8-carbon of the 1,2-naphthoquinone.

11. The system of claim 1, wherein R⁵ in at least one subunit of the polymer is Formula II, R⁸ in the at least one subunit is a tethered subunit of Formula I, R⁵ in the tethered subunit of Formula I is Formula II, and the tethered subunit of Formula I is tethered via R⁸ of the tethered subunit of Formula I.

12. The system of claim 1, wherein R⁵ in at least one subunit of the polymer is Formula III.

13. The system of claim 1, wherein the spacer arm of R⁴ comprises one or more instances of a moiety selected from the group consisting of —(CH₂)ₘ—, —(CH₂)ₘ—O—(CH₂)ₘ—, —(CH₂)ₘ—(NR¹⁸R¹⁹)—(CH₂)ₘ—, and combinations thereof, wherein:
R¹⁸ and R¹⁹ in each instance is independently H; alkyl, cycloalkyl, aryl, heterocycle, heteroaryl, or a combination thereof; or a nitrogen protecting group, with the proviso that at least one of R¹⁸ and R¹⁹ may be absent; and
each instance of m is independently 1-20.

14. The system of claim 1, wherein R⁵ in at least one subunit of the polymer is Formula II, wherein at least one of R⁶, R⁷, and R⁸ in Formula II is Formula IV: 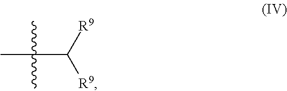
wherein R⁹ in each instance is independently hydrogen, alkyl, alkynyl, cycloalkyl, aryl, halogen-substituted aryl (including aryl groups substituted multiple times with the same or different halogen), nitro-substituted aryl, heterocycle, heteroaryl, hydroxyl, halo, haloalkyl, amino, an amino protected with a nitrogen protecting group, pyridine, ester, amide, azide, sulfate, sulfite, sulfoalkyl, sulfhydryl, sulfonamide, thiazole, nitro, cyano, alkoxy, carboxy, aldehyde, a saturated or unsaturated cycloalkyl, a saturated or unsaturated heterocycle, bridged saturated and unsaturated cylcoalkyl, fused aromatic, aromatic heterocycle, an N-hydroxysuccinimide ester-reactive group, an amine-reactive group, a sulfhydryl-reactive group, a tethered polypeptide, a tethered redox mediator, a linking arm; a tethered subunit of Formula I, or a combination thereof.

15. The system of claim 1, wherein the polymer further includes subunits of Formula V: 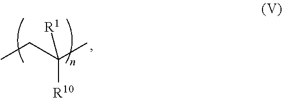
wherein, in each instance of Formula V, R¹ is H, alkyl, cycloalkyl, aryl, heterocycle, heteroaryl, or a combination thereof; n is a positive integer; and R¹⁰ is carboxyl or carboxamido.

16. The system of claim 1, wherein the polymer is provided in the form of a hydrogel.

17. The system of claim 16, wherein the hydrogel includes an untethered redox mediator compound.

18. The system of claim 16, further comprising an electrode in contact with the hydrogel.

19. A method of detecting an analyte, the method comprising contacting a sample containing the analyte with the system of claim 1, wherein:
R⁵ in at least one subunit is Formula II, R⁸ is a tethered polypeptide, and the polypeptide is an oxidoreductase; and
the detecting includes the oxidoreductase oxidizing the analyte.

20. The method of claim 19, wherein:
R⁵ in at least one subunit is Formula II and R⁸ is a tethered redox mediator; and
the detecting includes the redox mediator undergoing a redox reaction.

21. The method of claim 19, wherein:
the polymer is provided in the form of a hydrogel;
the system further includes an electrode in contact with the hydrogel; and
the detecting includes the electrode undergoing a change in electric charge.

22. The method of claim 19, wherein the analyte includes glucose.
